(19) United States
(12) Patent Application Publication
(10) Pub. No.: US 2011/0092491 A1
(43) Pub. Date: Apr. 21, 2011

(54) Compounds and Compositions as Inhibitors of Receptor Tyrosine Kinase Activity

(75) Inventors: Dai CHENG, San Diego, CA (US); Qiang DING, San Diego, CA (US); Dong HAN, San Diego, CA (US); Nathanael S. GRAY, San Diego, CA (US); Guobao ZHANG, San Diego, CA (US)

(73) Assignee: NOVARTIS AG, Basel (CH)

(21) Appl. No.: 12/976,187

(22) Filed: Dec. 22, 2010

Related U.S. Application Data
(63) Continuation of application No. 10/917,578, filed on Aug. 13, 2004, now abandoned.
(60) Provisional application No. 60/495,408, filed on Aug. 16, 2003.
(60) Provisional application No. 60/524,357, filed on Nov. 21, 2003.
(60) Provisional application No. 60/565,367, filed on Apr. 26, 2004.

(51) Int. Cl. A61K 31/551; C07D 473/16; C07D 413/14; C07D 243/08; A61K 31/52; A61K 31/5377; A61K 31/497; A61P 35/00; A61P 25/00

(52) U.S. Cl. 514/218; 544/277; 544/118; 540/575; 514/263.22; 514/263.2; 514/234.2; 514/255.05

(57) ABSTRACT

The invention provides a novel class of compounds, pharmaceutical compositions comprising such compounds and methods of using such compounds to treat or prevent diseases or disorders associated with cSRC, Lck, FGFR3, Flt3, TrkB, Bmx, and/or PFGFRα kinase activity.

### CROSS-REFERENCE TO RELATED APPLICATIONS

This application is a continuation of U.S. application Ser. No. 10/917,578 filed 13 Aug. 2004, which claims the benefit of priority to U.S. Provisional Patent Applications 60/495,406 filed 15 Aug. 2003; 60/524,357 filed 21 Nov. 2003; and 60/565,367 filed 26 Apr. 2004. The full disclosures of these applications are incorporated herein by reference in their entirety and for all purposes.

### BACKGROUND OF THE INVENTION

1. Field of the Invention

The invention provides a novel class of compounds, pharmaceutical compositions comprising such compounds and methods of using such compounds to treat or prevent diseases or disorders associated with cSRC, Lck, FGFR3, Flt3, TrkB, Bmx, and/or PFGFRα kinase activity.

2. Background

The protein kinases represent a large family of proteins, which play a central role in the regulation of a wide variety of cellular processes and maintaining control over cellular function. A partial, non-limiting, list of these kinases include: receptor tyrosine kinases such as Fms-like tyrosine kinase 3 (Flt3), platelet-derived growth factor receptor kinase (PDGF-R), the receptor kinase for stem cell factor, c-kit, the nerve growth factor receptor, trkB, and the fibroblast growth factor receptor (FGFR3); non-receptor tyrosine kinases such Abl and the fusion kinase BCR-Abl, Fes, Lck and Syk; and serine/threonine kinases such as b-RAF, MAP kinases (e.g., MKK6) and SAPK2β. Aberrant kinase activity has been observed in many disease states including benign and malignant proliferative disorders as well as diseases resulting from inappropriate activation of the immune and nervous systems.

The novel compounds of this invention inhibit the activity of one or more protein kinases and are, therefore, expected to be useful in the treatment of kinase-associated diseases.

### SUMMARY OF THE INVENTION

In one aspect, the present invention provides compounds of Formula I:

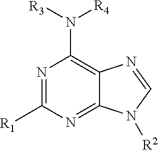

in which:

R₁ is selected from hydrogen, halo, C₁₋₆alkyl, halo-substituted-C₁₋₆alkyl, C₁₋₆alkoxy, halo-substituted-C₁₋₆alkoxy, —OXOR⁵, —OXR⁶, —OXNR₅R₆, —OXONR₅R₆, —OXR₆, —XNR₅R₆ and —XNR₇XNR₇R₇; wherein X is selected from a bond, C₁₋₆alkylene, C₂₋₆alkenylene and C₂₋₆alkynylene; wherein R₂ is independently selected from hydrogen or C₁₋₆alkyl;

R₅ is selected from hydrogen, C₁₋₆alkyl and —XOR₇; wherein X is selected from a bond, C₁₋₆alkylene, C₂₋₆alkenylene and C₂₋₆alkynylene; and R₂ is independently selected from hydrogen or C₁₋₆alkyl;

R₆ is selected from hydrogen, C₁₋₆alkyl, C₃₋₁₂cycloalkylC₀₋₄alkyl, C₃₋₈heterocycloalkylC₀₋₄alkyl, C₆₋₁₀arylC₀₋₄alkyl and C₅₋₁₀heteroarylC₀₋₄alkyl; or

R₅ and R₆ together with the nitrogen atom to which both R₅ and R₆ are attached form C₃₋₈heterocycloalkyl or C₅₋₈heteroaryl; wherein a methylene of any heterocycloalkyl formed by R₅ and R₆ can be optionally replaced by —C(O)— or —S(O)₂—;

wherein any aryl, heteroaryl, cycloalkyl or heterocycloalkyl of R₆ or the combination of R₅ and R₆ can be optionally substituted by 1 to 3 radicals independently selected from —XNR₇R₇, —XOR₇, —XNR₇R₇, —XC(O)NR₇R₇, —XNR₇C(O)R₇, —XOR₇, —XC(O)OR₇, —XC(O)R₇, C₁₋₆alkyl, C₃₋₈heterocycloalkyl, C₅₋₁₀heteroaryl, C₃₋₁₂cycloalkyl and C₆₋₁₀arylC₀₋₄alkyl; wherein any alkyl or alkylene of R₁ can optionally have a methylene replaced by a divalent radical selected from —NR₇C(O)—, —C(O)NR₇—, —NR₇—, —C(O)—, —O—, —S—, —S(O)— and —S(O)₂—; and wherein any alkyl or alkylene of R₆ can be optionally substituted by 1 to 3 radicals independently selected from C₅₋₈heteroaryl, —NR₇R₇, —C(O)NR₇R₇, —NR₇C(O)R₇, halo and hydroxy; wherein R₇ is independently selected from hydrogen or C₁₋₆alkyl;

R₂ is selected from hydrogen, C₆₋₁₀aryl and C₅₋₁₀heteroaryl; wherein any aryl or heteroaryl of R₂ is optionally substituted with 1 to 3 radicals independently selected from —XNR₇R₇, —XOR₇, —XOR₈, —XC(O)OR₇, —XC(O)R₇, C₁₋₆alkyl, C₁₋₆alkoxy, nitro, cyano, hydroxy, halo and halo-substituted-C₁₋₆alkyl; wherein X and R₇ are as described above; and R₈ is C₆₋₁₀arylC₀₋₄alkyl;

R₃ is selected from hydrogen and C₁₋₆alkyl;

R₄ is selected from C₃₋₁₂cycloalkylC₀₋₄alkyl, C₃₋₈heterocycloalkylC₀₋₄alkyl, C₆₋₁₀arylC₀₋₄alkyl and C₅₋₁₀heteroarylC₀₋₄alkyl; wherein any alkylene of R₄ can optionally have a methylene replaced by a divalent radical selected from —C(O)—, —S—, —S(O)— and —S(O)₂—; wherein said aryl, heteroaryl, cycloalkyl or heterocycloalkyl of R₄ is optionally substituted by 1 to 3 radicals selected from halo, C₁₋₆alkyl, C₁₋₆alkoxy, halo-substituted-C₁₋₆alkyl, halo-substituted-C₁₋₆alkoxy, —XR₉, —XOR₉, —XS(O)₀₋₂R₇, —XS(O)₀₋₂R₉, —XC(O)R₇, —XC(O)OR₇, —XP(O)R₇R₇, —XC(O)R₉, —XC(O)NR₇XNR₇R₇, —XC(O)NR₇R₇, —XC(O)NR₇R₉ and —XC(O)NR₇XOR₇; wherein X and R₇ are as described above; R₉ is selected from C₃₋₁₂cycloalkylC₀₋₄alkyl, C₃₋₈heterocycloalkylC₀₋₄alkyl, C₆₋₁₀aryl and C₅₋₁₀heteroaryl; wherein any aryl, heteroaryl, cycloalkyl or heterocycloalkyl of R₉ is optionally substituted by 1 to 3 radicals selected from C₁₋₆alkyl, —XC(O)R₇ and —XC(O)NR₇R₇; wherein X and R₇ are as described above; and the N-oxide derivatives, prodrug derivatives, protected derivatives, individual isomers and mixture of isomers thereof; and the pharmaceutically acceptable salts and solvates (e.g. hydrates) of such compounds.

In a second aspect, the present invention provides a pharmaceutical composition which contains a compound of Formula I or a N-oxide derivative, individual isomers and mixture of isomers thereof; or a pharmaceutically acceptable salt thereof, in admixture with one or more suitable excipients.

In a third aspect, the present invention provides a method of treating a disease in an animal in which inhibition of cSRC, Lck, FGFR3, Flt3, TrkB, PDGFRα and/or Bmx activity can prevent, inhibit or ameliorate the pathology and/or symptomology of the disease, which method comprises administering to the animal a therapeutically effective amount of a compound of Formula I or a N-oxide derivative, individual isomers and mixture of isomers thereof, or a pharmaceutically acceptable salt thereof.

In a fourth aspect, the present invention provides the use of a compound of Formula I in the manufacture of a medicament for treating a disease in an animal in which cSRC, Lck, FGFR3, Flt3, TrkB, PDGFRα and/or Bmx activity contributes to the pathology and/or symptomology of the disease.

In a fifth aspect, the present invention provides a process for preparing compounds of Formula I and the N-oxide derivatives, prodrug derivatives, individual isomers and mixture of isomers thereof, and the pharmaceutically acceptable salts thereof.

### DETAILED DESCRIPTION OF THE INVENTION

### Definitions

“Alkyl” as a group and as a structural element of other groups, for example halo-substituted-alkyl and alkoxy, can be either straight-chained or branched. C₁₋₄-alkoxy includes, methoxy, ethoxy, and the like. Halo-substituted alkyl includes trifluoromethyl, pentafluoroethyl, and the like.

“Aryl” means a monocyclic or fused bicyclic aromatic ring assembly containing six to ten ring carbon atoms. For example, aryl may be phenyl or naphthyl, preferably phenyl. “Arylene” means a divalent radical derived from an aryl group. “Heteroaryl” is as defined for aryl where one or more of the ring members are a heteroatom. For example heteroaryl includes pyridyl, indolyl, indazolyl, quinoxalinyl, quinolinyl, benzofuranyl, benzopyranyl, benzothiopyranyl, benzo[1,3]dioxole, imidazolyl, benzo-imidazolyl, pyrimidinyl, furanyl, oxazolyl, isoxazolyl, triazolyl, tetrazolyl, pyrazolyl, thienyl, etc.

“Cycloalkyl” means a saturated or partially unsaturated, monocyclic, fused bicyclic or bridged polycyclic ring assembly containing the number of ring atoms indicated. For example, C₃₋₁₀cycloalkyl includes cyclopropyl, cyclobutyl, cyclopentyl, cyclohexyl, etc. “Heterocycloalkyl” means cycloalkyl, as defined in this application, provided that one or more of the ring carbons indicated, are replaced by a moiety selected from —O—, —N═, —NR—, —C(O)—, —S—, —S(O)— or —S(O)₂—, wherein R is hydrogen, C₁₋₄alkyl or a nitrogen protecting group. For example, C₃₋₈heterocycloalkyl as used in this application to describe compounds of the invention includes morpholino, pyrrolidinyl, piperazinyl, piperidinyl, piperidinylone, 1,4-dioxa-8-aza-spiro[4.5]dec-8-yl, etc.

“Halogen” (or halo) preferably represents chloro or fluoro, but may also be bromo or iodo.

“Treat”, “treating” and “treatment” refer to a method of alleviating or abating a disease and/or its attendant symptoms. In the present description, the term “treatment” includes both prophylactic or preventative treatment as well as curative or disease suppressive treatment, including treatment of patients at risk of contracting the disease or suspected to have contracted the disease as well as ill patients. This term further includes the treatment for the delay of progression of the disease.

The term “curative” as used herein means efficacy in treating ongoing episodes involving deregulated Flt3 receptor tyrosine kinase activity.

The term “prophylactic” means the prevention of the onset or recurrence of diseases involving deregulated Flt3 receptor tyrosine kinase activity.

The term “delay of progression” as used herein means administration of the active compound to patients being in a pre-stage or in an early phase of the disease to be treated, in which patients for example a pre-form of the corresponding disease is diagnosed or which patients are in a condition, e.g. during a medical treatment or a condition resulting from an accident, under which it is likely that a corresponding disease will develop.

The term “diseases involving deregulated Flt3 receptor tyrosine kinase activity” as used herein includes, but is not limited to, leukemias including acute myeloid leukemia (AML), AML with trilineage myelodysplasia (AML/TMDS), acute lymphoblastic leukemia (ALL), and myelodysplastic syndrome (MDS). This term also, specifically includes diseases resulting from Flt3 receptor mutation.

### DESCRIPTION OF THE PREFERRED EMBODIMENTS

The invention provides a novel class of compounds, pharmaceutical compositions comprising such compounds and methods of using such compounds to treat or prevent diseases or disorders associated with cSRC, Lck, FGFR3, Flt3, TrkB, PDGFRα and/or Bmx kinase activity. In particular, the compounds show high potency toward the Flt3 and FGFR3 receptor kinases.

In one embodiment, with reference to compounds of Formula I:

R₁ is selected from hydrogen, halo, C₁₋₆alkoxy, —OXOR⁵, —OXR⁶, —OXNR₅R₆, —OXONR₅R₆, —XR₆, —XNR₇XNR₇R₇ and —XNR₅R₆; wherein X is selected from a bond, C₁₋₆alkylene, C₂₋₆alkenylene and C₂₋₆alkynylene;

R₅ is selected from hydrogen, C₁₋₆alkyl and —XOR₇; wherein X is selected from a bond, C₁₋₆alkylene, C₂₋₆alkenylene and C₂₋₆alkynylene; and R₇ is independently selected from hydrogen or C₁₋₆alkyl;

R₆ is selected from hydrogen, C₁₋₆alkyl, C₃₋₁₂cycloalkylC₀₋₄alkyl, C₃₋₈heterocycloalkylC₀₋₄alkyl, C₆₋₁₀arylC₀₋₄alkyl and C₅₋₁₀heteroarylC₀₋₄alkyl; R₆ is hydrogen or C₁₋₆alkyl; or

R₅ and R₆ together with the nitrogen atom to which both R₅ and R₆ are attached form C₃₋₈heterocycloalkyl or C₅₋₈heteroaryl; wherein a methylene of any heterocycloalkyl formed by R₅ and R₆ can be optionally replaced by —C(O)— and S(O)₂;

wherein any aryl, heteroaryl, cycloalkyl or heterocycloalkyl of R₆ or the combination of R₅ and R₆ can be optionally substituted by 1 to 3 radicals independently selected from —XNR₇R₇, —XC(O)NR₇R₇, —XOR₇, —XNR₇R₇, —XNR₇C(O)R₇, —XOR₇, —XC(O)R₇, C₁₋₆alkyl, C₃₋₈heterocycloalkyl and C₆₋₁₀arylC₀₋₄alkyl; wherein any alkyl or alkylene of R₁ can optionally have a methylene replaced by a divalent radical selected from —NR₇C(O)—, —C(O)NR₇—, —NR₇—, —O—; and wherein any alkyl or alkylene of R₁ can be optionally substituted by 1 to 3 radicals independently selected from C₅₋₈heteroaryl, —NR₇R₇, —C(O)NR₇R₇, —NR₇C(O)R₇, halo and hydroxy; wherein R₇ is independently selected from hydrogen or C₁₋₆alkyl;

R₂ is selected from hydrogen, C₆₋₁₀aryl and C₅₋₁₀heteroaryl; wherein any aryl or heteroaryl of R₂ is optionally substituted with 1 to 3 radicals independently selected from —XNR₇R₇, —XOR₇, —XOR₈, —XC(O)OR₇, C₁₋₆alkyl, C₁₋₆alkoxy, nitro, cyano, halo, halo-substituted-C₁₋₆alkoxy and halo-substituted-C₁₋₆alkyl; wherein X and R₇ are as described above; and R₈ is C₆₋₁₀arylC₀₋₄alkyl;

R₃ is hydrogen; and

R₄ is selected from C₆₋₁₀arylC₀₋₄alkyl and C₅₋₁₀heteroarylC₀₋₄alkyl; wherein said aryl or heteroaryl of R₄ is substituted by 1 to 3 radicals selected from halo, —XR₉, —XOR₉, —XS(O)₂R₇, —XS(O)₂R₉, —XC(O)R₇, —XC(O)OR₇, —XP(O)R₇R₇, —XC(O)R₉, —XC(O)NR₇XNR₇R₇, —XC(O)NR₇R₇, —XC(O)NR₇R₉ and —XC(O)NR₇XOR₇; wherein X and R₇ are as described above; R₉ is C₃₋₈heterocycloalkylC₀₋₄alkyl; wherein R₉ is optionally substituted by 1 to 3 radicals selected from C₁₋₆alkyl, —XC(O)R₇ and —XC(O)NR₇R₇; wherein X and R₇ are as described above.

In another embodiment, R₁ is selected from hydrogen, halo, C₁₋₆alkoxy, —OXOR⁵, —OXR⁶, —OXNR₅R₆, —OXONR₅R₆, —XR₆ and —XNR₅R₆; wherein X is selected from a bond, C₁₋₆alkylene, C₂₋₆alkenylene and C₂₋₆alkynylene; R₅ is selected from hydrogen, methyl, hydroxy-ethyl and methoxy-ethyl; R₆ is selected from hydrogen, phenyl, benzyl, cyclopentyl, cyclobutyl, dimethylamino-propenyl, cyclohexyl, 2,3-dihydroxy-propyl, piperidinyl, amino-carbonyl-ethyl, methyl-carbonyl-amino-ethyl, methyl-amino-ethyl, amino-propyl, methyl-amino-propyl, 1-hydroxymethyl-butyl, pentyl, butyl, propyl, methoxy-ethynyl, methoxy-ethenyl, dimethyl-amino-butyl, dimethyl-amino-ethyl, dimethyl-amino-propyl, tetrahydropyranyl, tetrahydrofuranyl-methyl, pyridinyl-methyl, a zepan-1-yl, [1,4]oxazepan-4-yl, piperidinyl-ethyl, diethyl-amino-ethyl, amino-butyl, amino-isopropyl, amino-ethyl, hydroxy-ethyl, 2-acetylamino-ethyl, carbamoyl-ethyl, 4-methyl-[1,4]diazepan-1-yl, 2-hydroxy-propyl, hydroxy-propyl, 2-hydroxy-2-methyl-propyl, methoxy-ethyl, amino-propyl, methyl-amino-propyl, 2-hydroxy-2-phenyl-ethyl, pyridinyl-ethyl, morpholino-propyl, morpholino-ethyl, pyrrolidinyl, pyrrolidinyl-methyl, pyrrolidinyl-ethyl, pyrrolidinyl-propyl, pyrazinyl, quinolin-3-yl, quinolin-5-yl, imidazolyl-ethyl, pyridinyl-methyl, phenethyl, tetrahydro-pyran-4-yl, pyrimidinyl, furanyl, isoxazolyl-methyl, pyridinyl, benzo[1,3]dioxol-5-yl, thiazolyl-ethyl and thiazolyl-methyl; or R₅ and R₆ together with the nitrogen atom to which both R₅ and R₆ are attached form pyrrolidinyl, piperazinyl, piperidinyl, imidazolyl, 3-oxo-piperazin-1-yl, [1,4]diazepan-1-yl, morpholino, 3-oxo-piperazin-1-yl, 1,1-dioxo-1λ⁶-thiomorpholin-4-yl or pyrazolyl;

wherein any aryl, heteroaryl, cycloalkyl or heterocycloalkyl of R₆ or the combination of R₅ and R₆ can be optionally substituted by 1 to 3 radicals independently selected from methyl-carbonyl, amino-methyl, amino-carbonyl, methyl-sulfonyl, methoxy, methoxy-methyl, formyl, fluoro-ethyl, hydroxy-ethyl, amino, dimethyl-amino, hydroxy, methyl, ethyl, acetyl, isopropyl, pyrrolidinyl, pyrimidinyl, morpholino, pyridinyl and benzyl; wherein any alkyl or alkylene of R₆ can optionally have a methylene replaced by a divalent radical selected from —NHC(O)— or —C(O)NH—; and wherein any alkyl or alkylene of R₆ can be optionally substituted by 1 to 2 radicals independently selected from amino, halo, piperidinyl and hydroxy.

In another embodiment, R₂ is selected from hydrogen, phenyl, thienyl, pyridinyl, pyrazolyl, thiazolyl, pyrazinyl, naphthyl, furanyl, benzo[1,3]dioxol-5-yl, isothiazolyl, imidazolyl and pyrimidinyl; wherein any aryl or heteroaryl of R₂ is optionally substituted with 1 to 3 radicals independently selected from methyl, isopropyl, halo, acetyl, trifluoromethyl, nitro, 1-hydroxy-ethyl, 1-hydroxy-1-methyl-ethyl, hydroxy-ethyl, hydroxy-methyl, formamyl, methoxy, benzyloxy, carboxy, amino, cyano, amino-carbonyl, amino-methyl and ethoxy.

In another embodiment, R₄ is selected from phenyl, benzyl, pyridinyl and 1-oxo-indan-5-yl; wherein said phenyl, benzyl, indanyl or pyridinyl is optionally substituted with halo, acetyl, trifluoromethyl, cyclopropyl-amino-carbonyl, azetidine-1-carbonyl, piperidinyl-carbonyl, morpholino, methyl-carbonyl, piperazinyl, methyl-sulfonyl, piperidinyl-sulfonyl, 4-methyl-piperazinyl-carbonyl, dimethyl-amino-ethyl-amino-carbonyl, morpholino-carbonyl, morpholino-methyl, amino-carbonyl, propyl-amino-carbonyl, hydroxy-ethyl-amino-carbonyl, morpholino-ethyl-amino-carbonyl, 4-acetyl-piperazine-1-carbonyl, 4-amino-carbonyl-piperazine-1-carbonyl, phenyl-carbonyl, pyrrolidinyl-1-carbonyl, propyl-carbonyl, butyl, isopropyl-oxy-carbonyl, cyclohexyl-carbonyl, cyclopropyl-carbonyl, methyl-sulfonyl, dimethyl-phosphinoyl, 4-methyl-piperazinyl-sulfonyl, 1-oxo-indan-5-yl, oxetane-3-sulfonyl, amino-sulphonyl and tetrahydro-pyran-4-sulfonyl.

Preferred compounds of Formula I are detailed in the Examples and Tables 1, 2 and 3, below. Further preferred examples are selected from: N⁶-(4-Methanesulfinyl-phenyl)-N²-methyl-N²-(tetrahydro-pyran-4-yl)-9-thiazol-4-yl-9H-purine-2,6-diamine; (4-Methanesulfonyl-phenyl)-[2-(2-methyl-morpholin-4-yl)-9-thiazol-4-yl-9H-purin-6-yl]-amine; 1-{4-[2-(2-Methyl-morpholin-4-yl)-9-thiazol-4-yl-9H-purin-6-ylamino]-phenyl}-ethanone; [4-(Dimethyl-phosphinoyl)-phenyl]-[2-(2-methyl-morpholin-4-yl)-9-thiazol-4-yl-9H-purin-6-yl]-amine; Azetidin-1-yl-{4-[2-(4-morpholin-4-yl-piperidin-1-yl)-9-thiazol-4-yl-9H-purin-6-ylamino]-phenyl}-methanone; 1-(4-{2-[Methyl-(1-methyl-piperidin-4-yl)-amino]-9-thiazol-4-yl-9H-purin-6-ylamino}-phenyl)-ethanone; 1-{4-[2-(2-Methyl-morpholin-4-yl)-9-thiophen-3-yl-9H-purin-6-ylamino]-phenyl}-ethanone; (4-Methanesulfonyl-phenyl)-[2-(4-morpholin-4-yl-piperidin-1-yl)-9-thiazol-4-yl-9H-purin-6-yl]-amine; N⁶-(4-Methanesulfonyl-phenyl)-N²-methyl-N²-(1-methyl-piperidin-4-yl)-9-thiazol-4-yl-9H-purine-2,6-diamine; [2-(2-Methyl-morpholin-4-yl)-9-thiazol-4-yl-9H-purin-6-yl]-(4-morpholin-4-yl-phenyl)-amine; N²-Methyl-N²-(1-methyl-piperidin-4-yl)-N⁶-(4-morpholin-4-yl-phenyl)-9-thiazol-4-yl-9H-purine-2,6-diamine; N²-Methyl-N²-(1-methyl-piperidin-4-yl)-N⁶-(4-morpholin-4-yl-phenyl)-9-thiophen-3-yl-9H-purine-2,6-diamine; [2-(2,2-Dimethyl-morpholin-4-yl)-9-thiazol-4-yl-9H-purin-6-yl]-(4-methanesulfonyl-phenyl)-amine; [2-(2,6-Dimethyl-morpholin-4-yl)-9-thiazol-4-yl-9H-purin-6-yl]-(4-methanesulfonyl-phenyl)-amine; [4-(Dimethyl-phosphinoyl)-phenyl]-[2-(2-ethyl-morpholin-4-yl)-9-thiophen-3-yl-9H-purin-6-yl]-amine; [4-(Dimethyl-phosphinoyl)-phenyl]-[2-(2-fluoromethyl-morpholin-4-yl)-9-thiophen-3-yl-9H-purin-6-yl]-amine; [2-(2,6-Dimethyl-morpholin-4-yl)-9-thiazol-4-yl-9H-purin-6-yl]-[4-(dimethyl-phosphinoyl)-phenyl]-amine; [2-(2,6-Dimethyl-morpholin-4-yl)-9-thiophen-3-yl-9H-purin-6-yl]-[4-(dimethyl-phosphinoyl)-phenyl]-amine; [4-(Dimethyl-phosphinoyl)-phenyl]-[2-(2-methyl-morpholin-4-yl)-9-thiophen-3-yl-9H-purin-6-yl]-amine; [4-(Dimethyl-phosphinoyl)-phenyl]-[2-(3-methyl-piperidin-1-yl)-9-thiazol-4-yl-9H-purin-6-yl]-amine; N⁶-(4-Methanesulfonyl-phenyl)-N²-methyl-N²-pyridin-2-ylmethyl-9-thiophen-3-yl-9H-purine-2,6-diamine; N²-Methyl-N⁶-(4-morpholin-4-yl-phenyl)-N²-pyridin-2-ylmethyl-9-thiophen-3-yl-9H-purine-2,6-diamine; (2-Azepan-1-yl-9-thiazol-4-yl-9H-purin-6-yl)-[4-(dimethyl-phosphinoyl)-phenyl]-amine; N²-Cyclohexyl-N⁶-[4-(dimethyl-phosphinoyl)-phenyl]-N²-methyl-9-thiazol-4-yl-9H-purine-2,6-diamine; N⁶-(4-Methanesulfonyl-phenyl)-N²-methyl-N²-(tetrahydro-pyran-4-yl)-9-thiazol-4-yl-9H-purine-2,6-diamine; N⁶-(4-Methanesulfonyl-phenyl)-N²-pyridin-2-ylmethyl-9-thiazol-4-yl-9H-purine-2,6-diamine; N²-Cyclohexyl-N⁶-(4-methanesulfinyl-phenyl)-N²-methyl-9-thiazol-4-yl-9H-purine-2,6-diamine; R-(4-Methanesulfinyl-phenyl)-[2-(2-methyl-morpholin-4-yl)-9-thiazol-4-yl-9H-purin-6-yl]-amine; N⁶-(4-Methanesulfonyl-phenyl)-N²-methyl-N²-pyridin-2-ylmethyl-9-thiazol-4-yl-9H-purine-2,6-diamine; {4-[6-(4-Methanesulfonyl-phenylamino)-2-(methyl-pyridin-2-ylmethyl-amino)-purin-9-yl]-phenyl}-methanol; R-(4-Methanesulfonyl-phenyl)-[2-(2-methyl-morpholin-4-yl)-9-thiazol-4-yl-9H-purin-6-yl]-amine; R-4-[2-(2-Methyl-morpholin-4-yl)-9-thiazol-4-yl-9H-purin-6-ylamino]-benzenesulfonamide; and {4-[6-(4-Methanesulfonyl-phenylamino)-2-(2-methyl-morpholin-4-yl)-purin-9-yl]-phenyl}-methanol.

### Pharmacology and Utility

Compounds of the invention inhibit the activity of Flt3 receptor tyrosine kinases and, as such, are useful for treating diseases or disorders in which FLT3 activity contribute to the pathology and/or symptomology of the disease.

Flt3 is a member of the type III receptor tyrosine kinase (RTK) family. Flt3 (fms-like tyrosine kinase) is also known as FLk-2 (fetal liver kinase 2). Aberrant expression of the Flt3 gene has been documented in both adult and childhood leukemias including acute myeloid leukemia (AML), AML with trilineage myelodysplasia (AML/TMDS), acute lymphoblastic leukemia (ALL), and myelodysplastic syndrome (MDS). Activating mutations of the Flt3 receptor have been found in about 35% of patients with acute myeloblastic leukemia (AML), and are associated with a poor prognosis. The most common mutation involves in-frame duplication within the juxtamembrane domain, with an additional 5-10% of patients having a point mutation at asparagine 835. Both of these mutations are associated with constitutive activation of the tyrosine kinase activity of Flt3, and result in proliferation and viability signals in the absence of ligand. Patients expressing the mutant form of the receptor have been shown to have a decreased chance for cure. Thus, there is accumulating evidence for a role for hyper-activated (mutated) Flt3 kinase activity in human leukemias and myelodysplastic syndrome. This has prompted the applicant to search for new inhibitors of the Flt3 receptor as a possible therapeutic approach in these patients, for whom current drug therapies offer little utility, and for such patients who have previously failed current available drug therapies and/or stem cell transplantation therapies.

Leukemias generally result from an acquired (not inherited) genetic injury to the DNA of immature hematopoietic cells in the bone marrow, lymph nodes, spleen, or other organs of the blood and immune system. The effects are: the accelerated growth and blockage in the maturation of cells, resulting in the accumulation of cells called “leukemic blasts”, which do not function as normal blood cells; and a failure to produce normal marrow cells, leading to a deficiency of red cells (anemia), platelets and normal white cells. Blast cells are normally produced by bone marrow and usually develop into mature blood cells, comprising about 1 percent of all marrow cells. In leukemia, the blasts do not mature properly and accumulate in the bone marrow. In acute myeloid leukemia (AML), these are called myeloblasts while in acute lymphoblastic leukemia (ALL) they are known as lymphoblasts. Another leukemia is mixed-lineage leukemia (MLL).

The term “AML with trilineage myelodysplasia (AML/TMDS)” relates to an uncommon form of leukemia characterized by a dyshematopoietic picture accompanying the acute leukemia, a poor response to induction chemotherapy, and a tendency to relapse with pure myelodysplastic syndrome.

The term “Myelodysplastic Syndrome (MDS)” relates to a group of blood disorders in which the bone marrow stops functioning normally, resulting in a deficiency in the number of healthy blood cells. Compared with leukemia, in which one type of blood cell is produced in large numbers, any and sometimes all types of blood cells are affected in MDS. At least 10,000 new cases occur annually in the United States. Up to one third of patients diagnosed with MDS go on to develop acute myeloid leukemia. For this reason the disease is sometimes referred to as preleukemia. Myelodysplastic syndrome is sometimes also called myelodysplasia dysmyelopoiesis or oligoblastic leukemia. MDS is also referred to as smoldering leukemia when high numbers of blast cells remain in the marrow.

Myelodysplastic syndrome, like leukemia, results from a genetic injury to the DNA of a single cell in the bone marrow. Certain abnormalities in chromosomes are present in MDS patients. These abnormalities are called translocations, which occur when a part of one chromosome breaks off and becomes attached to a broken part of a different chromosome. The same defects are frequently found in acute myeloid leukemia. However, MDS differs from leukemia because all of the patient's blood cells are abnormal and all are derived from the same damaged stem cell. In leukemia patients, the bone marrow contains a mixture of diseased and healthy blood cells.

AML and advanced myelodysplastic syndromes are currently treated with high doses of cytotoxic chemotherapy drugs such cytosine arabinoside and daunorubicin. This type of treatment induces about 70% of patients to enter a hematological remission. However, more than half of the patients that enter remission will later relapse despite administration of chemotherapy over long periods of time. Almost all of the patients who either fail to enter remission initially, or relapse later after obtaining remission, will ultimately die because of leukemia. Bone marrow transplantation can cure up to 50-60% of patients who undergo the procedure, but only about one third of all patients with AML or MDS are eligible to receive a transplant. New and effective drugs are urgently needed to treat the patients who fail to enter remission with standard therapies, patients who later relapse, and patients that are not eligible for stem cell transplantation. Further, an effective new drug could be added to standard therapy with the reasonable expectation that it will result in improved induction chemotherapy for all patients.

FGFR3 is part of a family of structurally related tyrosine kinase receptors encoded by 4 different genes. Specific point mutations in different domains of the FGFR3 gene lead to constitutive activation of the receptor and are associated with autosomal dominant skeletal disorders, multiple myeloma, and a large proportion of bladder and cervical cancer (Cappellen, et al, Nature, vol. 23). Activating mutations placed in the mouse FGFR3 gene and the targeting of activated FGFR3 to growth plate cartilage in mice result in dwarfism. Analogous to our concept, targeted disruption of FGFR3 in mice results in the overgrowth of long bones and vertebrae. In addition, 20-25% of multiple myeloma cells contain a t(4; 14)(p16.3; q32.3) chromosomal translocation with breakpoints on 4p16 located 50-100 kb centromeric to FGFR3. In rare cases of multiple myeloma, activating mutations of FGFR3 previously seen in skeletal disorders have been found and are always accompanied by this chromosomal translocation. Recently, FGFR3 missense somatic mutations (R248C, S249C, G372C, and K652E) have been identified in a large proportion of bladder cancer cells and in some cervical cancer cells, and these in fact are identical to the germinal activating mutations that cause thanatophoric dysplasia, a form of dwarfism lethal in the neonatal period. Compounds of the invention can have therapeutic utility for multiple myeloma by being more effective than current treatment, for bladder cancer by avoiding life-altering cystectomy, and for cervical cancer in those patients who wish to preserve future fertility.

Compounds of the present invention, can be used not only as a tumor-inhibiting substance, for example in small cell lung cancer, but also as an agent to treat non-malignant proliferative disorders, such as atherosclerosis, thrombosis, psoriasis, scleroderma and fibrosis, as well as for the protection of stem cells, for example to combat the hemotoxic effect of chemotherapeutic agents, such as 5-fluoruracil, and in asthma. Compounds of the invention can especially be used for the treatment of diseases, which respond to an inhibition of the PDGF receptor kinase.

Compounds of the present invention show useful effects in the treatment of disorders arising as a result of transplantation, for example, allogenic transplantation, especially tissue rejection, such as especially obliterative bronchiolitis (OB), i.e. a chronic rejection of allogenic lung transplants. In contrast to patients without OB, those with OB often show an elevated PDGF concentration in bronchoalveolar lavage fluids.

Compounds of the present invention are also effective in diseases associated with vascular smooth-muscle cell migration and proliferation (where PDGF and PDGF-R often also play a role), such as restenosis and atherosclerosis. These effects and the consequences thereof for the proliferation or migration of vascular smooth-muscle cells in vitro and in vivo can be demonstrated by administration of the compounds of the present invention, and also by investigating its effect on the thickening of the vascular intima following mechanical injury in vivo.

The trk family of neurotrophin receptors (trkA, trkB, trkC) promotes the survival, growth and differentiation of the neuronal and non-neuronal tissues. The TrkB protein is expressed in neuroendocrine-type cells in the small intestine and colon, in the alpha cells of the pancreas, in the monocytes and macrophages of the lymph nodes and of the spleen, and in the granular layers of the epidermis (Shibayama and Koizumi, 1996). Expression of the TrkB protein has been associated with an unfavorable progression of Wilms tumors and of neuroblastomas. TkrB is, moreover, expressed in cancerous prostate cells but not in normal cells. The signaling pathway downstream of the trk receptors involves the cascade of MAPK activation through the Shc, activated Ras, ERK-1 and ERK-2 genes, and the PLC-gammal transduction pathway (Sugimoto et al., 2001).

The kinase, c-Src transmits oncogenic signals of many receptors. For example, over-expression of EGFR or HER2/neu in tumors leads to the constitutive activation of c-src, which is characteristic for the malignant cell but absent from the normal cell. On the other hand, mice deficient in the expression of c-src exhibit an osteopetrotic phenotype, indicating a key participation of c-src in osteoclast function and a possible involvement in related disorders.

Fibroblast growth factor receptor 3 was shown to exert a negative regulatory effect on bone growth and an inhibition of chondrocyte proliferation. Thanatophoric dysplasia is caused by different mutations in fibroblast growth factor receptor 3, and one mutation, TDII FGFR3, has a constitutive tyrosine kinase activity which activates the transcription factor Stat1, leading to expression of a cell-cycle inhibitor, growth arrest and abnormal bone development (Su et al., Nature, 1997, 386, 288-292). FGFR3 is also often expressed in multiple myeloma-type cancers.

Lck plays a role in T-cell signaling. Mice that lack the Lck gene have a poor ability to develop thymocytes. The function of Lck as a positive activator of T-cell signaling suggests that Lck inhibitors may be useful for treating autoimmune disease such as rheumatoid arthritis.

In accordance with the foregoing, the present invention further provides a method for preventing or treating any of the diseases or disorders described above in a subject in need of such treatment, which method comprises administering to said subject a therapeutically effective amount of a compound of Formula I or a pharmaceutically acceptable salt thereof. For any of the above uses, the required dosage will vary depending on the mode of administration, the particular condition to be treated and the effect desired.

### Administration and Pharmaceutical Compositions

In general, compounds of the invention will be administered in therapeutically effective amounts via any of the usual and acceptable modes known in the art, either singly or in combination with one or more therapeutic agents. A therapeutically effective amount may vary widely depending on the severity of the disease, the age and relative health of the subject, the potency of the compound used and other factors. In general, satisfactory results are indicated to be obtained systemically at daily dosages of from about 0.03 to 2.5 mg/kg per body weight. An indicated daily dosage in the larger mammal, e.g. humans, is in the range from about 0.5 mg to about 100 mg, conveniently administered, e.g. in divided doses up to four times a day or in retard form. Suitable unit dosage forms for oral administration comprise from ca. 1 to 50 mg active ingredient.

Compounds of the invention can be administered as pharmaceutical compositions by any conventional route, in particular enterally, e.g., orally, e.g., in the form of tablets or capsules, or parenterally, e.g., in the form of injectable solutions or suspensions, topically, e.g., in the form of lotions, gels, ointments or creams, or in a nasal or suppository form. Pharmaceutical compositions comprising a compound of the present invention in free form or in a pharmaceutically acceptable salt form in association with at least one pharmaceutically acceptable carrier or diluent can be manufactured in a conventional manner by mixing, granulating or coating methods. For example, oral compositions can be tablets or gelatin capsules comprising the active ingredient together with a) diluents, e.g., lactose, dextrose, sucrose, mannitol, sorbitol, cellulose and/or glycine; b) lubricants, e.g., silica, talcum, stearic acid, its magnesium or calcium salt and/or polyethyleneglycol; for tablets also c) binders, e.g., magnesium aluminum silicate, starch paste, gelatin, tragacanth, methylcellulose, sodium carboxymethylcellulose and or polyvinylpyrrolidone; if desired d) disintegrants, e.g., starches, agar, alginic acid or its sodium salt, or effervescent mixtures; and/or e) absorbents, colorants, flavors and sweeteners. Injectable compositions can be aqueous isotonic solutions or suspensions, and suppositories can be prepared from fatty emulsions or suspensions. The compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Suitable formulations for transdermal applications include an effective amount of a compound of the present invention with a carrier. A carrier can include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin. Matrix transdermal formulations may also be used. Suitable formulations for topical application, e.g., to the skin and eyes, are preferably aqueous solutions, ointments, creams or gels well-known in the art. Such may contain solubilizers, stabilizers, tonicity enhancing agents, buffers and preservatives.

Compounds of the invention can be administered in therapeutically effective amounts in combination with one or more therapeutic agents (pharmaceutical combinations) including radiation and bone marrow transplantation. Non-limiting examples of compounds which can be used in combination with compounds of the invention are cytotoxic chemotherapy drugs, such as cytosine arabinoside, daunorubicin, cyclophosphamide, VP-16, mitoxantrone, daunorubicin, cytarabine, methotrexate, vincristine, 6-thioguanine, 6-mercaptopurine, paclitaxel etc., an anti-angiogenic agent, such as, but not limited to a cyclooxygenase inhibitor such as celecoxib, immunomodulatory or anti-inflammatory substances, for example, cyclosporin, rapamycin, or ascomycin, or immunosuppressant analogues thereof, for example cyclosporin A (CsA), cyclosporin G, FK-506, rapamycin, or comparable compounds, corticosteroids, cyclophosphamide, azathioprine, methotrexate, brequinar, leflunomide, mizoribine, mycophenolic acid, mycophenolate mofetil, 15-deoxyspergualin, immunosuppressant antibodies, especially monoclonal antibodies for leukocyte receptors, for example MHC, CD2, CD3, CD4, CD7, CD25, CD28, B7, CD45, CD58 or their ligands, or other immunomodulatory compounds, such as CTLA41g. Further, compounds of the invention can be combined with other inhibitors of signal transduction or other oncogene-targeted drugs to produce significant synergistic therapies.

Where the compounds of the invention are administered in conjunction with other therapies, dosages of the co-administered compounds will of course vary depending on the type of co-drug employed, on the specific drug employed, on the condition being treated and so forth.

The invention also provides for a pharmaceutical combinations, e.g. a kit, comprising a) a first agent which is a compound of the invention as disclosed herein, in free form or in pharmaceutically acceptable salt form, and b) at least one co-agent. The kit can comprise instructions for its administration.

The terms “co-administration” or “combined administration” or the like as utilized herein are meant to encompass administration of the selected therapeutic agents to a single patient, and are intended to include treatment regimens in which the agents are not necessarily administered by the same route of administration or at the same time.

The term “pharmaceutical combination” as used herein means a product that results from the mixing or combining of more than one active ingredient and includes both fixed and non-fixed combinations of the active ingredients. The term “fixed combination” means that the active ingredients, e.g. a compound of Formula I and a co-agent, are both administered to a patient simultaneously in the form of a single entity or dosage. The term “non-fixed combination” means that the active ingredients, e.g. a compound of Formula I and a co-agent, are both administered to a patient as separate entities either simultaneously, concurrently or sequentially with no specific time limits, wherein such administration provides therapeutically effective levels of the 2 compounds in the body of the patient. The latter also applies to cocktail therapy, e.g. the administration of 3 or more active ingredients.

### Processes for Making Compounds of the Invention

The present invention also includes processes for the preparation of compounds of the invention. In the reactions described, it can be necessary to protect reactive functional groups, for example hydroxy, amino, imino, thio or carboxy groups, where these are desired in the final product, to avoid their unwanted participation in the reactions. Conventional protecting groups can be used in accordance with standard practice, for example, see T. W. Greene and P. G. M. Wuts in “Protective Groups in Organic Chemistry”, John Wiley and Sons, 1991.

Compounds of Formula I, in which R₅ is hydrogen, can be prepared by proceeding as in the following Reaction Scheme I:

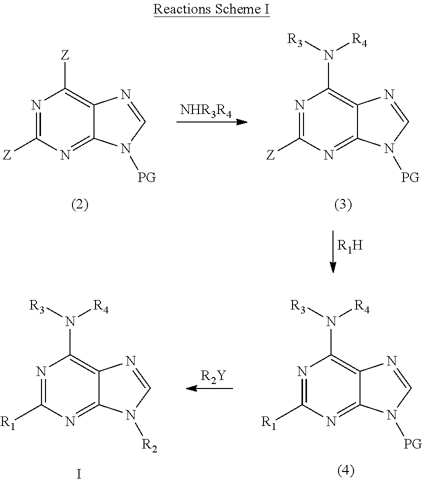

in which R₁, R₂, R₃ and R₄ are as defined for Formula I in the Summary of the Invention, PG represents a nitrogen protecting group (e.g., tetrahydro-pyran-2-yl, and the like), and Z represents a halo group, for example iodo or chloro, preferably chloro.

Compounds of Formula 3 can be prepared by reacting a compound of formula 2 with NHR₃R₄ in the presence of a suitable solvent (e.g., ethanol, butanol, THF and the like) using an appropriate base (e.g., DIEA, Na₂CO₃ and the like). Compounds of formula 4 can be prepared by reacting a compound of formula 3 with R₁H in the presence of a suitable solvent (e.g., DME, ethanol, butanol, THF and the like), optionally an appropriate catalyst (e.g., a Palladium catalyst or the like) and using an appropriate base (e.g., DIEA, Na₂CO₃ and the like). Compounds of Formula I can be prepared by first removing the protecting group (PG) in the presence of a suitable catalyst (e.g. p-TSA, or the like) in a suitable solvent (e.g., MeOH, or the like). The reaction further proceeds by reacting a deprotected compound of formula 4 with R₂Y, wherein Y represents a halo group, for example iodo, bromo or chloro. The reaction proceeds in the presence of a suitable solvent (e.g., DMF, dioxane or the like) using an appropriate base (e.g., Potassium Phosphate or the like), at a temperature range of about 70 to about 110° C. and can take up to 24 hours to complete.

Compounds of Formula I can be prepared by proceeding as in the following Reaction Scheme II:

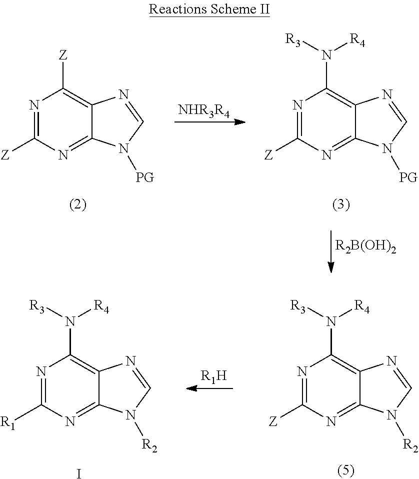

in which R₁, R₂, R₃ and R₄ are as defined for Formula I in the Summary of the Invention, PG represents a nitrogen protecting group (e.g., tetrahydro-pyran-2-yl or the like), and Z represents a halo group, for example iodo or chloro, preferably chloro.

Compounds of Formula 3 can be prepared by reacting a compound of formula 2 with NHR₃R₄ in the presence of a suitable solvent (e.g., ethanol, butanol, THF or the like) using an appropriate base (e.g., DIEA, Na₂CO₃ or the like). Compounds of formula 5 can be prepared by first removing the protecting group (PG) in the presence of a suitable catalyst (e.g. p-TSA, or the like) in a suitable solvent (e.g., MeOH, or the like). The reaction further proceeds by reacting a deprotected compound of formula 3 with R₂B(OH)₂ in the presence of a suitable solvent (e.g., dioxane, methylene chloride, and the like) and a suitable catalyst (e.g. copper acetate, or the like) using an appropriate base (e.g., pyridine, TEA, or the like).

The reaction proceeds in the temperature range of about 20 to about 80° C. and can take up to 168 hours to complete. Compounds of Formula I can be prepared by reacting a compound of formula 5 with R₁H in the presence of a suitable solvent (e.g., butanol, ethanol and the like) using an appropriate base (e.g., DIEA, Na₂CO₃ or the like).

Compounds of Formula I can be prepared by proceeding as in the following Reaction Scheme III:

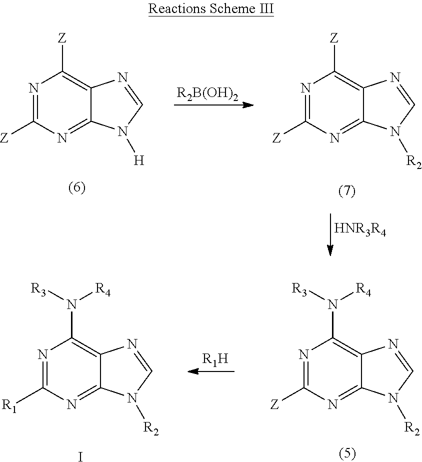

in which R₁, R₂, R₃ and R₄ are as defined for Formula I in the Summary of the Invention and Z represents a halo group, for example iodo or chloro, preferably chloro.

Compounds of formula 7 can be prepared by reacting a compound of formula 6 with R₂B(OH)₂ in the presence of a suitable solvent (e.g., dioxane, methylene chloride and the like) and a suitable catalyst (e.g. copper acetate, or the like) using an appropriate base (e.g., pyridine, TEA or the like). The reaction proceeds in the temperature range of about 20 to about 80° C. and can take up to 168 hours to complete. Compounds of formula 5 can be prepared by reacting a compound of formula 7 with NHR₃R₄ in the presence of a suitable solvent (e.g., DME, ethanol, butanol, THF and the like), optionally with an appropriate catalyst (e.g., a palladium catalyst or the like) and using an appropriate base (e.g., DIEA, Na₂CO₃ or the like). Compounds of Formula I can be prepared by reacting a compound of formula 5 with R₁H in the presence of a suitable solvent (e.g., butanol, ethanol, THF and the like) using an appropriate base (e.g., DIEA, Na₂CO₃ or the like).

### Additional Processes for Making Compounds of the Invention

A compound of the invention can be prepared as a pharmaceutically acceptable acid addition salt by reacting the free base form of the compound with a pharmaceutically acceptable inorganic or organic acid. Alternatively, a pharmaceutically acceptable base addition salt of a compound of the invention can be prepared by reacting the free acid form of the compound with a pharmaceutically acceptable inorganic or organic base. Alternatively, the salt forms of the compounds of the invention can be prepared using salts of the starting materials or intermediates.

The free acid or free base forms of the compounds of the invention can be prepared from the corresponding base addition salt or acid addition salt from, respectively. For example a compound of the invention in an acid addition salt form can be converted to the corresponding free base by treating with a suitable base (e.g., ammonium hydroxide solution, sodium hydroxide, and the like). A compound of the invention in a base addition salt form can be converted to the corresponding free acid by treating with a suitable acid (e.g., hydrochloric acid, etc.)

Compounds of the invention in unoxidized form can be prepared from N-oxides of compounds of the invention by treating with a reducing agent (e.g., sulfur, sulfur dioxide, triphenyl phosphine, lithium borohydride, sodium borohydride, phosphorus trichloride, tribromide, or the like) in a suitable inert organic solvent (e.g. acetonitrile, ethanol, aqueous dioxane, or the like) at 0 to 80° C.

Prodrug derivatives of the compounds of the invention can be prepared by methods known to those of ordinary skill in the art (e.g., for further details see Saulnier et al., (1994), Bioorganic and Medicinal Chemistry Letters, Vol. 4, p. 1985). For example, appropriate prodrugs can be prepared by reacting a non-derivatized compound of the invention with a suitable carbamylating agent (e.g., 1,1-acyloxyalkylcarbanochloridate, para-nitrophenyl carbonate, or the like).

Protected derivatives of the compounds of the invention can be made by means known to those of ordinary skill in the art. A detailed description of techniques applicable to the creation of protecting groups and their removal can be found in T. W. Greene, “Protecting Groups in Organic Chemistry”, 3^{rd} edition, John Wiley and Sons, Inc., 1999.

Compounds of the present invention can be conveniently prepared, or formed during the process of the invention, as solvates (e.g., hydrates). Hydrates of compounds of the present invention can be conveniently prepared by recrystallization from an aqueous/organic solvent mixture, using organic solvents such as dioxin, tetrahydrofuran or methanol.

Compounds of the invention can be prepared as their individual stereoisomers by reacting a racemic mixture of the compound with an optically active resolving agent to form a pair of diastereoisomeric compounds, separating the diastereomers and recovering the optically pure enantiomers. While resolution of enantiomers can be carried out using covalent diastereomeric derivatives of the compounds of the invention, dissociable complexes are preferred (e.g., crystalline diastereomeric salts). Diastereomers have distinct physical properties (e.g., melting points, boiling points, solubilities, reactivity, etc.) and can be readily separated by taking advantage of these dissimilarities. The diastereomers can be separated by chromatography, or preferably, by separation/resolution techniques based upon differences in solubility. The optically pure enantiomer is then recovered, along with the resolving agent, by any practical means that would not result in racemization. A more detailed description of the techniques applicable to the resolution of stereoisomers of compounds from their racemic mixture can be found in Jean Jacques, Andre Collet, Samuel H. Wilen, “Enantiomers, Racemates and Resolutions”, John Wiley And Sons, Inc., 1981.

In summary, the compounds of Formula I can be made by a process, which involves:

(a) those of reaction schemes I, II and III, for example coupling compounds of formula 5 with R₁H according to reaction schemes II or III; and

(b) optionally converting a compound of the invention into a pharmaceutically acceptable salt;

(c) optionally converting a salt form of a compound of the invention to a non-salt form;

(d) optionally converting an unoxidized form of a compound of the invention into a pharmaceutically acceptable N-oxide;

(e) optionally converting an N-oxide form of a compound of the invention to its unoxidized form;

(f) optionally resolving an individual isomer of a compound of the invention from a mixture of isomers;

(g) optionally converting a non-derivatized compound of the invention into a pharmaceutically acceptable prodrug derivative; and

(h) optionally converting a prodrug derivative of a compound of the invention to its non-derivatized form.

Insofar as the production of the starting materials is not particularly described, the compounds are known or can be prepared analogously to methods known in the art or as disclosed in the Examples hereinafter.

One of skill in the art will appreciate that the above transformations are only representative of methods for preparation of the compounds of the present invention, and that other well known methods can similarly be used.

### EXAMPLES

The following examples provide detailed descriptions of the preparation of representative compounds and are offered to illustrate, but not to limit the present invention.

### Example 1

### {4-[2-(4-Amino-cyclohexylamino)-9-phenyl-9H-purin-6-ylamino]-phenyl}-piperidin-1-yl-methanone

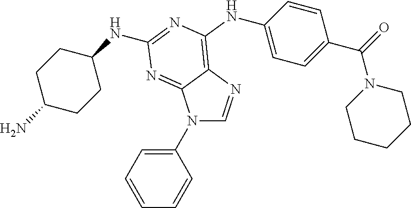

To a solution of piperidine (18.0 g, 211.8 mmol) in dichloromethane (360 mL) at 0° C. is added 4-nitrobenzoyl chloride (18.6 g, 100 mmol) cautiously in several portions. The reaction mixture is stirred at room temperature for 10 minutes before it is washed with HCl (1%, 2×200 mL) solution and water (300 mL) and dried with Na₂SO₄. After evaporation of the solvent, (4-nitro-phenyl)-piperidin-1-yl-methanone (23.2 g, 99%) is obtained and used directly in hydrogenation (1.0 g of 10% Pd/C in 400 mL of ethanol). After filtration of the catalyst and evaporation of ethanol, (4-amino-phenyl)-piperidin-1-yl-methanone (19.6 g, 96%) is obtained.

A mixture of 2,6-dichloropurine (18.80 g, 100 mmol), 3,4-dihydro-2H-pyran (12.62 g, 150 mmol), p-toluenesulfonic acid monohydrate (1.90 g, 10 mmol) and anhydrous dichloromethane (200 mL) is stirred at room temperature for 4 hours. After filtration, it is washed with Na₂CO₃ (10% aqueous, 100 mL), water (100 mL) and dried with Na₂SO₄. Evaporation of the solvent followed by titration with ethyl acetate (5 mL) and hexanes (60 mL) induces precipitate which upon filtration yields 2,6-dichloro-9-(tetrahydro-pyran-2-yl)-9H-purine (24.01 g, 88%).

The mixture of 2,6-dichloro-9-(tetrahydro-pyran-2-yl)-9H-purine (5.44 g, 20 mmol), (4-amino-phenyl)-piperidin-1-yl-methanone (4.08 g, 20 mmol), diisopropylethylamine (24 mmol) and ethanol (100 mL) are refluxed for 24 hours. Then trans-1,4-cyclohexanediamine (6.84 g, 60 mmol) and diisopropylethylamine (24 mmol) are added and the mixture is refluxed for another 24 hours. The oily residue obtained after evaporation of ethanol is treated with ethyl acetate (250 mL) and water (200 mL). The aqueous phase is extracted with ethyl acetate (2×100 mL) and the combined organic phase dried with Na₂SO₄. After evaporation, the oily residue obtained is treated with p-toluenesulfonic acid monohydrate (3.80 g, 20 mmol) in methanol (100 mL) at 55° C. for 4 hours and the reaction monitored until deprotection is completed.

Diisopropylethylamine is added to neutralize the mixture. The oily residue obtained is subjected to column chromatography (EtOAc:MeOH=9:1, then CH₂Cl₂:MeOH (containing ˜7N ammonia)=9:1) to give 2-(4-amino-cyclohexylamino)-6-[4-(piperidine-1-carbonyl)-phenylamino]-9H-purine (6.50 g, 75%).

A reaction vial containing a mixture of 2-(4-amino-cyclohexylamino)-6-[4-(piperidine-1-carbonyl)-phenylamino]-9H-purine (86.8 mg, 0.2 mmol) prepared as above, copper(I) iodide (38.2 mg, 0.2 mmol) and potassium phosphate (170 mg, 0.8 mmol) is degassed and refilled with dry nitrogen. N,N′-Dimethylethylenediamine (35.3 mg, 43 μL, 0.4 mmol) and iodobenzene (40.8 mg, 0.2 mmol) in DMF (700 μL) are added and the mixture is stirred at 88° C. overnight. AcOH-MeOH (1:10, 1.5 mL) is added to neutralize the mixture followed by filtration through a syringe filter. Column chromatography (EtOAc:MeOH=9:1, then CH₂Cl₂:MeOH (containing ˜7N ammonia)=9:1) yields {4-[2-(4-amino-cyclohexylamino)-9-phenyl-9H-purin-6-ylamino]-phenyl}-piperidin-1-yl-methanone as a solid; ¹H NMR 400 MHz (CD₃OD) d 8.03 (s, 1H), 7.90-7.95 (m, 2H), 7.75-7.65 (m, 2H), 7.50-7.42 (m, 2H), 7.38-7.30 (m, 3H), 3.80-3.50 (m, 5H), 2.83-2.73 (m, 1H), 2.15-2.05 (m, 2H), 1.95-1.90 (m, 2H), 1.70-1.40 (m, 6H), 1.40-1.20 (m, 4H); MS m/z 511.3 (M+1).

### Example 2

### [4-(2-Chloro-9-phenyl-9H-purin-6-ylamino)-phenyl]-piperidin-1-yl-methanone

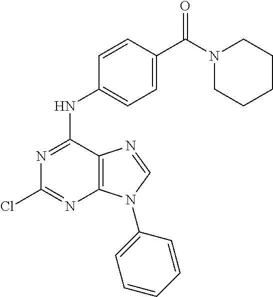

A mixture of 2,6-dichloro-9-(tetrahydra-pyran-2-yl)-9H-purine (10 g, 36.6 mmol), (4-amino-phenyl)-piperidin-1-yl-methanone (7.48 g, 36.6 mmol) and diisopropylethylamine (9.5 g, 73.5 mmol) in ethanol (110 ml) is refluxed overnight. The mixture is cooled down to room temperature and concentrated in vacuo to give [4-(2-chloro-9H-purin-6-ylamino)-phenyl]-piperidin-1-yl-methanone (14.7 g, 91%) as a dark yellow solid.

A mixture of [4-(2-chloro-9H-purin-6-ylamino)-phenyl]-piperidin-1-yl-methanone (10 g, 22.7 mmol) and p-toluenesulfonic acid monohydrate (0.86 g, 4.5 mmol) in methanol (100 mL) is stirred for 2 hours at 50° C. The mixture is cooled down to room temperature and suspended in methanol. The precipitate is collected and washed with ethyl acetate to give [4-(2-chloro-9H-purin-6-ylamino)-phenyl]-piperidin-1-yl-methanone (7.69 g, 95%) as a pale yellow solid.

To a suspension of activated molecular sieves (4.2 g) in dioxane (35 mL) is added [4-(2-chloro-9H-purin-6-ylamino)-phenyl]-piperidin-1-yl-methanone (4 g, 11.2 mmol), phenyl boronic acid (2.73 g, 22.4 mmol), copper acetate (3.05 g, 16.8 mmol) and pyridine (3.54 g, 44.8 mmol). The mixture is stirred at room temperature overnight and then heated at 40° C. for 5 hours. The mixture is cooled down to room temperature, diluted with THF (50 mL), filtered through Celite and washed with methanol. The filtrate is concentrated under reduced pressure and the residue is purified by flash column chromatography (MeOH/dichloromethane=1/50) to give [4-(2-chloro-9-phenyl-9H-purin-6-ylamino)-phenyl]-piperidin-1-yl-methanone (3.89 g, 80%) as a yellow solid; ¹H NMR 400 MHz (CDCl₃) d 8.17 (s, 1H), 8.06 (s, 1H), 7.93 (d, 2H, J=8.8 Hz), 7.69 (d, 2H, J=8.8 Hz), 7.58 (d, 2H, J=8 Hz), 7.49 (t, 3H, J=7.2 Hz), 7.41 (d, 1H, J=7.2 Hz), 2.93-2.90 (m, 4H), 2.18-1.96 (m, 2H), 1.58-1.53 (m, 4H), 1.35-1.29 (m, 2H); MS m/z 433.2 (M+1).

### Example 3

### {4-[2-(3-Dimethylamino-pyrrolidin-1-yl)-9-phenyl-9H-purin-6-ylamino]-phenyl}-piperidin-1-yl-methanone

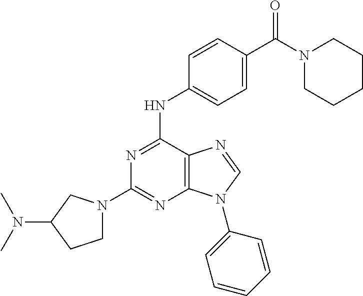

A mixture of [4-(2-chloro-9-phenyl-9H-purin-6-ylamino)-phenyl)]-piperidin-1-ylmethanone (129 mg, 0.3 mmol) and 3-(dimethylamino)-pyrrolidine (103 mg, 0.9 mmol) in 1-butanol (0.6 mL) is stirred for 12 hours at 120° C. The mixture is cooled to room temperature and concentrated under reduced pressure. The residue is purified by flash column chromatography (MeOH/dichloromethane=1/50) to give {4-[2-(3-dimethylamino-pyrrolidin-1-yl)-9-phenyl-9H-purin-6-ylamino]-phenyl}-piperidin-1-yl-methanone (73.3 mg, 49%) as a dark pink solid; ¹H NMR 400 MHz (MeOH-d₄) d 8.22 (s, 1H), 7.95 (d, 2H, J=8.4 Hz), 7.83 (d, 2H, J=7.6 Hz), 7.53 (t, 2H, J=7.6 Hz), 7.43 (d, 1H, J=7.6 Hz), 7.40 (d, 2H, J=8.8 Hz), 4.04-3.96 (m, 1H), 3.94-3.83 (m, 1H), 3.70-3.36 (m, 6H), 2.95 (s, 6H), 2.51-2.46 (m, 1H), 2.25-2.19 (m, 1H), 1.78-1.47 (m, 6H); MS m/z 511.3 (M+1).

### Example 4

### 4-(2-Imidazol-1-yl-9-phenyl-9H-purin-6-ylamino)-phenyl]piperidin-1-yl-methanone

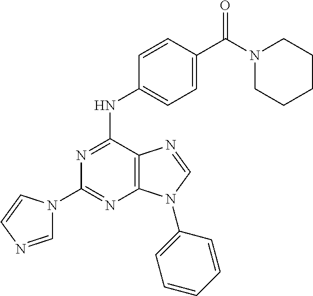

In a quartz reaction vessel (2 mL) is added [4-(2-chloro-9-phenyl-9H-purin-6-yl-amino)-phenyl)]piperidin-1-ylmethanone (43 mg, 0.1 mmol) and imidazole (20.4 mg, 0.3 mmol) in NMP (0.3 mL). The reaction vessel is then placed into the cavity of a microwave reactor (Emrys optimizer) and irradiated for 30 minutes at 200° C. The crude reaction mixture is purified by preparative HPLC to give the trifluoroacetate salt of 4-(2-imidazol-1-yl-9-phenyl-9H-purin-6-ylamino)-phenyl]piperidin-1-yl-methanone (18.7 mg) as a pale yellow solid; ¹H NMR 400 MHz (MeOH-d₄) d 9.52 (s, 1H), 8.58 (s, 1H), 8.26 (s, 1H), 7.91 (d, 2H, J=6.8 Hz), 7.86 (d, 2H, J=8.8 Hz), 7.65 (m, 3H), 7.56 (d, 1H, J=7.6 Hz), 7.51 (d, 2H, J=8.8 Hz), 3.70-3.49 (m, 4H), 1.77-1.60 (m, 6H); MS m/z 465.3 (M+1).

### Example 5

### {4-[9-Phenyl-2-(quinolin-3-ylamino)-9H-purin-6-ylamino]-phenyl}-piperidin-1-yl-methanone

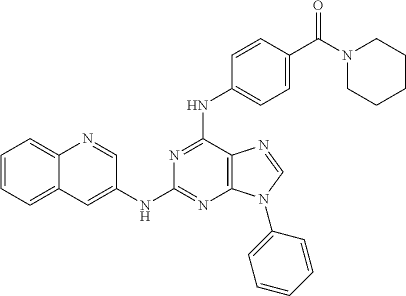

A tube is charged with [4-(2-chloro-9-phenyl-9H-purin-6-ylamino)-phenyl)]-piperidin-1-ylmethanone (43 mg, 0.1 mmol), 3-aminoquinoline (21.6 mg, 0.15 mmol), tris(dibenzylideneacetone) dipalladium (0) (7 mg, 0.008 mmol), 2-(di-t-butylphosphino) biphenyl (8.9 mg, 0.03 mmol), potassium phosphate (100 mg, 0.47 mmol), evacuated, and backfilled with nitrogen. DME (0.7 mL) is added under nitrogen. The reaction mixture is stirred at 85° C. for 16 hours. The resulting pale brown suspension is cooled down to room temperature and purified by preparative HPLC to give the trifluoroacetate salt of {4-[9-phenyl-2-(quinolin-3-ylamino)-9H-purin-6-ylamino]-phenyl}-piperidin-1-yl-methanone (24.5 mg) as a yellow solid; ¹H NMR 400 MHz (MeOH-d₄) d 9.29 (d, 1H, J=2.4 Hz), 9.13 (d, 1H, J=2.0 Hz), 8.18 (s, 1H), 7.92 (d, 1H, J=8.4 Hz), 7.81-7.70 (m, 7H), 7.58 (t, 2H, J=8.0 Hz), 7.48 (t, 1H, J=7.2 Hz), 7.30 (d, 2H, J=8.4 Hz), 3.87-3.35 (m, 4H), 1.80-1.43 (m, 6H); MS m/z 541.3 (M+1).

### Example 6

### N²-(4-Amino-cyclohexyl)-N⁶-(4-morpholin-4-yl-phenyl)-9-phenyl-9H-purine-2,6-diamine

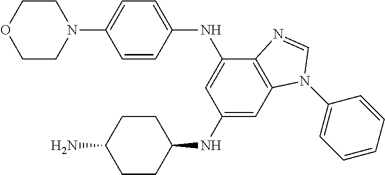

Molecular sieve (4A, 12.0 g) is dried under vacuum overnight at 150° C. and cooled down to room temperature. Then 2-fluoro-6-chloro-purine (6.0 g, 35 mmol), phenylboronic acid (8.3 g, 70 mmol), copper acetate (9.0 g, 52 mmol) and triethylamine (19 mL, 140 mmol) are added and mixed in dry dioxane (100 mL). The reaction mixture is stirred at room temperature for 2 days with a drying tube attached. After the reaction is complete, the reaction mixture is diluted in methylene chloride (200 mL), filtered through a Celite pad and washed with methylene chloride (200 mL). The organic phase is combined and the solvent is removed by rotary evaporation. The crude product is purified by flash silica gel column chromatography using hexanes/ethyl acetate (2:1) as eluent, to give 2-fluoro-6-chloro-9-phenyl-9H-purine (2.1 g, 24%) as light yellow solid, MS m/z 249.1 (M+1).

2-Fluoro-6-chloro-9-phenyl-9H-purine (50 mg, 0.20 mmol), 4-morpholin-4-yl-phenylamine (39 mg, 0.22 mmol) and diisopropylethylamine (35 μL, 0.2 mmol) are mixed in 1-butanol (0.4 mL). The reaction is stirred at 80° C. for 2 hours before trans-1,4-cyclohexanediamine (68 mg, 0.6 mmol) and diisopropylethylamine (70 μL, 0.4 mmol) are added. The reaction mixture is stirred at 110° C. overnight. The solvent is removed by rotary evaporation. The crude mixture is redissolved in DMSO and purified by HPLC to give the trifluoroacetate salt of N²-(4-amino-cyclohexyl)-N⁶-(4-morpholin-4-yl-phenyl)-9-phenyl-9H-purine-2,6-diamine as a white powder; ¹H NMR 400 MHz (DMSO-d₆) δ 9.29 (s, 1H), 8.23 (s, 1H), 7.84 (t, 4H, J=9.4 Hz), 7.51 (t, 2H, J=8.0 Hz), 7.35 (t, 1H, J=7.2 Hz), 6.84 (d, 2H, J=9.2 Hz), 6.48 (d, 1H, J=7.2 Hz), 3.71 (t, 4H, J=4.8 Hz), 3.57 (s, 1H), 3.01 (t, 4H, J=4.8 Hz), 1.93 (d, 2H, J=12 Hz), 1.77 (d, 2H, J=11.2 Hz), 1.24 (m, 4H), 0.90 (t, 1H, J=7.2 Hz); MS m/z 485.3 (M+1).

### Example 7

### N²-(4-Amino-cyclohexyl)-N⁶-[3-(4-methyl-piperazin-1-yl)-phenyl]-9-phenyl-9H-purine-2,6-diamine

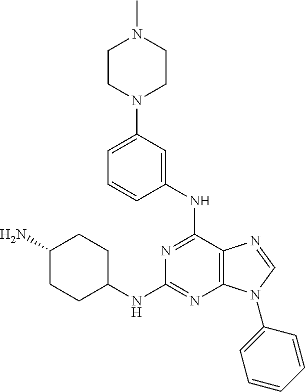

1-Chloro-3-nitro-benzene (1.0 g, 7 mmol) is mixed with 1-methyl-piperazine (2.0 mL) and the reaction is capped and stirred at 190° C. for 2 hours. After reaction, the excess 1-methyl-piperazine is removed by rotary evaporation to give the crude product as yellow oil. The crude product is purified by silica gel flash column to give 1.2 g of 1-methyl-4-(3-nitro-phenyl)-piperazine (yield 78%).

The 1-methyl-4-(3-nitro-phenyl)-piperazine (1.2 g, 5.4 mmol) is dissolved in methanol (50 mL) and Pd/C (5%, 120 mg) is added to the solution. A hydrogen balloon is attached to the flask. The solution is stirred overnight at room temperature. After the reaction is complete, the Pd/C is filtered and the filtrate collected and concentrated by rotary evaporation, to give 3-(4-methyl-piperazin-1-yl)-phenylamine.

2-Fluoro-6-chloro-9-phenyl-9H-purine (50 mg, 0.20 mmol), 3-(4-methyl-piperazin-1-yl)-phenylamine (42 mg, 0.22 mmol) and diisopropylethylamine (35 μL, 0.2 mmol) are mixed in 1-butanol (0.4 mL). The reaction is stirred at 80° C. for 2 hours before adding trans-1,4-cyclohexanediamine (68 mg, 0.6 mmol) and diisopropylethylamine (70 μL, 0.4 mmol). The reaction mixture is stirred at 110° C. overnight. The solvent is removed by rotary evaporation and the crude product is redissolved in DMSO and purified by HPLC to give N²-(4-amino-cyclohexyl)-N⁶-[3-(4-methyl-piperazin-1-yl)-phenyl]-9-phenyl-9H-purine-2,6-diamine as a white powder; ¹H NMR 400 MHz (DMSO-d₆) δ 9.12 (s, 1H), 8.16 (s, 1H), 7.78 (d, 2H, J=6.0 Hz), 7.58 (d, 1H, J=7.6 Hz), 7.42 (m, 2H), 7.24 (m, 2H), 7.00 (t, 1H, J=8.0 Hz), 6.48 (m, 2H), 3.53 (s, 1H), 3.25 (m, 4H), 3.01 (t, 4H, J=4.8 Hz), 2.09 (s, 3H), 1.74 (m, 2H), 1.66 (s, 2H), 0.92 (m, 4H), 0.79 (t, 1H, J=7.2 Hz); MS m/z 498.3 (M+1).

### Example 8

### 1-{4-[2-(2-Methyl-morpholin-4-yl)-9-thiazol-4-yl-9H-purin-6-ylamino]-phenyl}-ethanone

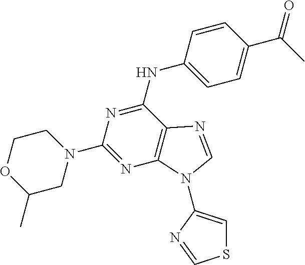

1-(4-Amino-phenyl)-ethanone (1.0 g, 7.4 mmol) is mixed with 2-fluoro-6-chloro-9-(tetrahydro-pyran-2-yl)-9H-purine (1.90 g, 7.4 mmol), diisopropylethylamine (1.54 mL, 8.9 mmol) and n-butanol 50 mL. The reaction is stirred in 95° C. for 14 hours. After cooling down to the room temperature and removing the solvent, the crude product is purified by flash chromatography using MeOH/DCM (5%:95%) to get 1-{4-[2-Fluoro-9-(tetrahydro-pyran-2-yl)-9H-purin-6-ylamino]-phenyl}-ethanone white solid 2.49 g.

1-{4-[2-Fluoro-9-(tetrahydro-pyran-2-yl)-9H-purin-6-ylamino]-phenyl}-ethanone (100 mg, 0.28 mmol) is mixed with 2-methyl-morpholine HCl salt (58 mg, 0.45 mmol), diisopropylethylamine (121 μL, 0.70 mmol) and 5 mL n-butanol. The reaction is stirred in 100° C. for 14 hours. After cooling down and remove the solvent, the crude product is purified by flash chromatography using EA/Hexane (1:1) to get 1-{4-[2-(2-Methyl-morpholin-4-yl)-9-(tetrahydro-pyran-2-yl)-9H-purin-6-ylamino]-phenyl}-ethanone yellow solid 115 mg.

1-{4-[2-(2-Methyl-morpholin-4-yl)-9-(tetrahydro-pyran-2-yl)-9H-purin-6-ylamino]-phenyl}-ethanone (115 mg, 0.26 mmol) is dissolved in 10 mL ethanol and mixed with 200 μL TFA. The reaction is stirred in 60° C. for 2 hours. After cooling down to the room temperature and totally removing the solvent and TFA, the crude product is mixed with copper (I) iodide (50 mg, 0.26 mmol) and potassium phosphate (220 mg, 0.8 mmol) and degassed and refilled with dry nitrogen. N,N′-Dimethylethylenediamine (46 mg, 0.52 mmol) and iodo-thiazole (53 mg, 0.26 mmol) in DMF (4 mL) are added and the mixture is stirred at 90° C. for 14 hours. After cooling down to room temperature, AcOH-MeOH (1:10, 1.6 mL) is added to neutralize the mixture followed by filtration through a syringe filter. After removing the solvent, the crude product is dissolved in DMSO and purified by preparative HPLC to get the pale solid 1-{4-[2-(2-Methyl-morpholin-4-yl)-9-thiazol-4-yl-9H-purin-6-ylamino]-phenyl}-ethanone 71 mg. ¹H NMR 600 MHz (DMSO-d₆) δ 10.21 (s, 1H), 9.26 (d, 1H, J=2.2), 8.60 (s, 1H), 8.27 (d, 1H, J=2.0 Hz), 8.07 (d, 2H, J=8.8 Hz), 7.95 (d, 2H, J=8.8 Hz), 4.50 (dd, 2H, J=3.0 Hz), 3.95 (dd, 1H, J=2.6 Hz), 3.59 (m, 2H), 3.04 (m, 1H), 2.72 (m, 1H), 2.54 (s, 3H), 1.22 (d, 3H, J=6.2 Hz); MS m/z 436.2 (M+1).

### Example 9

### (4-Methanesulfonyl-phenyl)-[2-(4-morpholin-4-yl-piperidin-1-yl)-9-thiazol-4-yl-9H-purin-6-yl]-amine

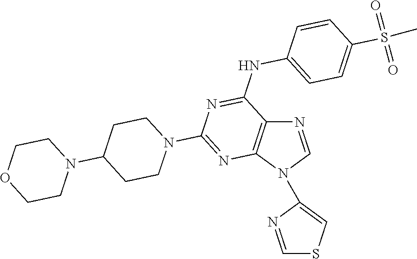

4-Methanesulfonyl-phenylamine (1.27 g, 7.4 mmol) is mixed with 2-fluoro-6-chloro-9-(tetrahydro-pyran-2-yl)-9H-purine (1.90 g, 7.4 mmol), diisopropylethylamine (1.54 mL, 8.9 mmol) and n-butanol 50 mL. The reaction is stirred in 95° C. for 14 hours. After cooling down to the room temperature and removing the solvent, the crude product is purified by flash chromatography using MeOH/DCM (7%:93%) to get [2-Fluoro-9-(tetrahydro-pyran-2-yl)-9H-purin-6-yl]-(4-methanesulfonyl-phenyl)-amine white solid 2.75 g.

[2-Fluoro-9-(tetrahydro-pyran-2-yl)-9H-purin-6-yl]-(4-methanesulfonyl-phenyl)-amine (110 mg, 0.28 mmol) is mixed with 4-Piperidin-4-yl-morpholine (76 mg, 0.45 mmol), diisopropylethylamine (121 μL, 0.70 mmol) and 5 mL n-butanol. The reaction is stirred in 100° C. for 14 hours. After cooling down and remove the solvent, the crude product is purified by flash chromatography using EA/Hexane (6:4) to get (4-Methanesulfonyl-phenyl)-[2-(4-morpholin-4-yl-piperidin-1-yl)-9-(tetrahydro-pyran-2-yl)-9H-purin-6-yl]-amine yellow solid 145 mg.

(4-Methanesulfonyl-phenyl)-[2-(4-morpholin-4-yl-piperidin-1-yl)-9-(tetrahydro-pyran-2-yl)-9H-purin-6-yl]-amine (145 mg, 0.26 mmol) is dissolved in 10 mL ethanol and mixed with 200 μL TFA. The reaction is stirred in 60° C. for 2 hours. After cooling down to the room temperature and totally removing the solvent and TFA, the crude product is mixed with copper (I) iodide (50 mg, 0.26 mmol) and potassium phosphate (220 mg, 0.8 mmol) and degassed and refilled with dry nitrogen. N,N′-Dimethylethylenediamine (46 mg, 0.52 mmol) and iodo-thiazole (53 mg, 0.26 mmol) in DMF (4 mL) are added and the mixture is stirred at 90° C. for 14 hours. After cooling down to room temperature, AcOH-MeOH (1:10, 1.6 mL) is added to neutralize the mixture followed by filtration through a syringe filter. After removing the solvent, the crude product is dissolved in DMSO and purified by preparative HPLC to get the white solid (4-Methanesulfonyl-phenyl)-[2-(4-morpholin-4-yl-piperidin-1-yl)-9-thiazol-4-yl-9H-purin-6-yl]-amine 95 mg. ¹H NMR 400 MHz (DMSO-d₆) δ 10.44 (s, 1H), 9.41 (s, 1H), 8.72 (s, 1H), 8.40 (d, 1H, J=2.4 Hz), 8.31 (d, 2H, J=8.8 Hz), 8.01 (d, 2H, J=8.0 Hz), 4.86 (d, 2H, J=12.8 Hz), 3.71 (s, 4H), 3.52 (m, 4H), 3.33 (s, 3H), 3.15 (t, 2H, J=12.0 Hz), 2.06 (d, 2H, J=11.2 Hz), 1.55 (m, 2H); MS m/z 541.3 (M+1).

### Example 10

### N⁶-(4-Methanesulfonyl-phenyl)-N²-pyridin-2-ylmethyl-9-thiazol-4-yl-9H-purine-2,6-diamine

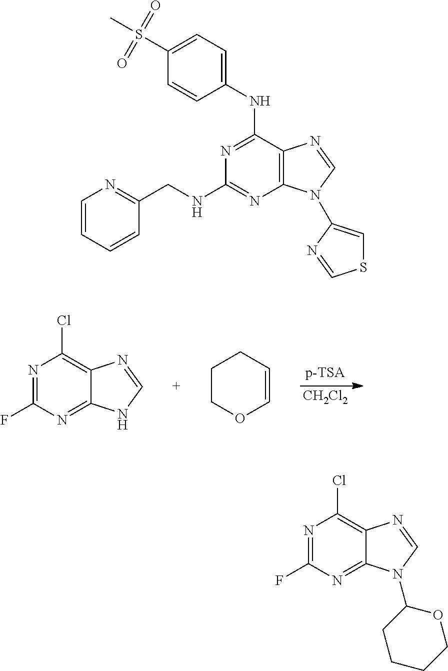

A mixture of 2-fluoro-6-chloropurine (17.26 g, 100 mmol), 3,4-dihydro-2H-pyran (12.62 g, 150 mmol) and p-toluenesulfonic acid monohydrate (1.90 g, 10 mmol) are dissolved in anhydrous dichloromethane (200 mL) and stirred at room temperature for 4 hours. The reaction mixture is filtered, washed with Na₂CO₃ (10% aqueous solution, 100 mL) and water (100 mL) and the organic layer dried with Na₂SO₄. Evaporation of the solvent results in an oil which is triturated with ethyl acetate (10 mL) and hexanes (60 mL) which induces precipitate formation. The product, 2-fluoro-6-chloro-9-(tetrahydro-pyran-2-yl)-9H-purine, is collected by filtration.

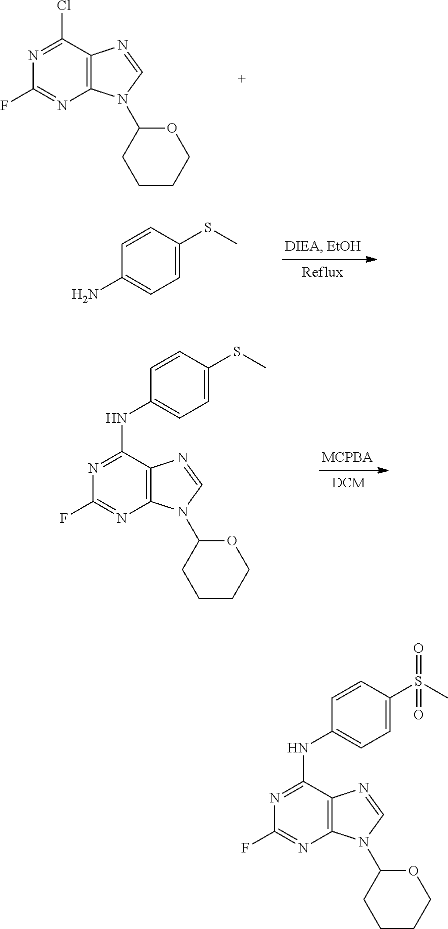

A mixture of 2-fluoro-6-chloro-9-(tetrahydro-pyran-2-yl)-9H-purine (2.56 g, 10 mmol), 4-(methylthio)aniline (1.39 g, 10 mmol) and DIEA (1.93 g, 15 mmol) in ethanol (20 ml) is stirred overnight at 78° C. The mixture is cooled down to room temperature. Evaporation of the solvent followed by column chromatography (EtOAc/DCM from 10% to 30%) yields [2-Fluoro-9-(tetrahydro-pyran-2-yl)-9H-purin-6-yl]-(4-methylsulfanyl-phenyl)-amine as a white solid.

To a solution of the compound obtained above (3.33 g, 9.25 mmol) in DCM (10 ml) is added 3-chloroperoxybenzoic acid (6.22 g, 77% maximum, 27.8 mmol) portion wise slowly (in an ice bath). After addition, the mixture is stirred at room temperature for another 2 hours. The mixture is diluted with DCM (50 ml) and the suspension is washed with saturated Na₂S₂O₃ (50 ml) and saturated NaHCO₃ (50 ml×2) until the organic phase is clear. The organic layer is further washed with water (50 ml) and brine (50 ml) and dried with MgSO₄. Evaporation of the solvent followed by column chromatography (EtOAc/DCM from 30% to 70%) gives [2-fluoro-9-(tetrahydro-pyran-2-yl)-9H-purin-6-yl]-(4-methylsulfonyl-phenyl)-amine as a pale yellow solid.

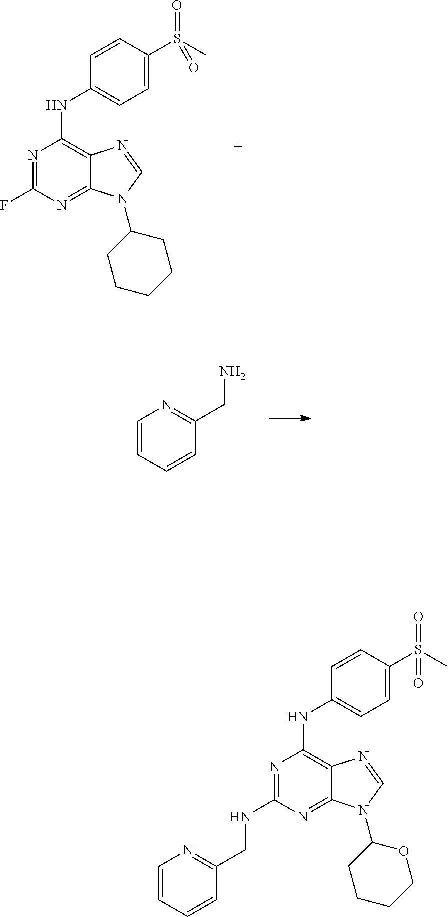

The mixture of the 2-fluoropurine substrate (4.6 g, 11.8 mmol) and 2-(aminomethyl)pyridine (15.0 g) is heated in an 84° C. oil bath, overnight. The mixture is distributed between ethyl acetate (200 mL) and water (200 mL). The organic phase is washed with NH₄Cl (2×150 mL, saturated aqueous solution) and water (200 mL) and dried over Na₂SO₄. Evaporation of the solvent gives the crude product which is used in the next reaction without further purification.

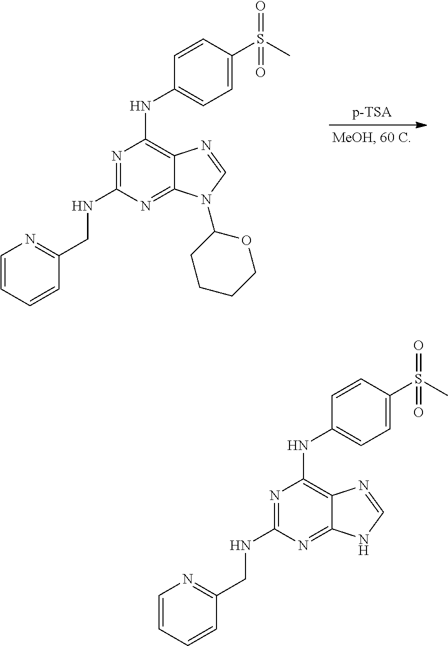

The compound obtained above (1.93 g, 4.02 mmol) is stirred with p-toluenesulfonic acid monohydrate (950 mg, 5.0 mmol) in methanol (20 mL) at 60° C. until the starting material is no longer be detected (monitored by TLC or LC-MS). Triethylamine (1.0 mL) is added. As the reaction mixture is cooled to room temperature precipitate forms which is collected by filtration to give the deprotected product.

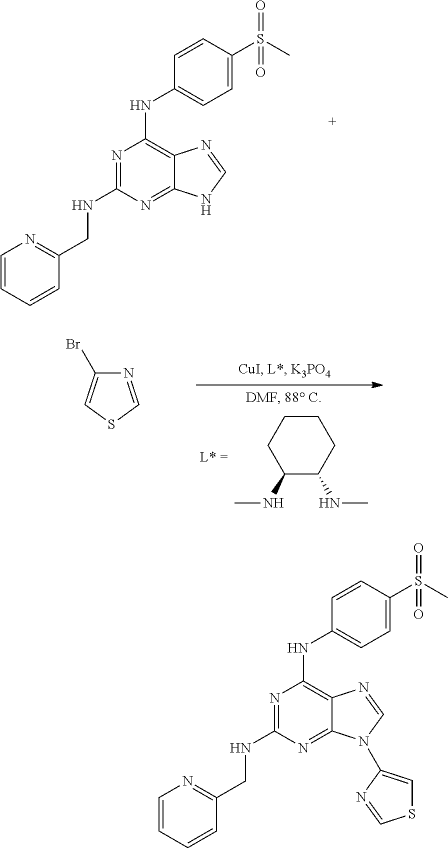

The deprotected 2,6-disubstituted purine (1.98 g, 5.0 mmol), CuI (475 mg, 2.50 mmol) and K₃PO₄ (3.18 g, 15 mmol) are combined in a flask (backfilled with argon). Trans-N,N′-dimethylcyclohexane-1,2-diamine (355 mg, 2.50 mmol) and 4-bromothiazole (932 mg, 88% pure, 5.0 mmol) in DMF (9.0 mL) is added and the mixture is stirred at 88° C. overnight. After the mixture is cooled to room temperature, acetic acid (1.0 mL) is added and the mixture is filtered through a syringe filter (washed with DMF). The filtrate purified by reverse-phase preparative LC-MS (acetonitrile/water/TFA gradient 10-90% CH₃CN in 7.5 minutes, Ultro 120 5 μM C18Q, 75×30 mmID). The collected water/MeCN solution of the product is evaporated to remove the acetonitrile. NaHCO₃ (saturated aqueous solution) is added to raise the pH to 9. DCM is used to extract the product and the organic phase is dried with Na₂SO₄. Evaporation of the solvent yielded the product as free base, N⁶-(4-Methanesulfonyl-phenyl)-N²-pyridin-2-ylmethyl-9-thiazol-4-yl-9H-purine-2,6-diamine as a white powder; ¹H NMR 400 MHz (d-DMSO) δ 10.21 (s, 1H), 9.26 (s, 1H), 8.53-7.70 (m, 9H), 7.42 (d, 1H, J=8.0 Hz,), 7.24 (t, 1H, J=6.0 Hz), 4.67 (d, 2H, J=5.6 Hz), 3.17 (s, 3H); MS m/z 479.3 (M+1).

### Example 11

### R-(4-Methanesulfonyl-phenyl)-[2-(2-methyl-morpholin-4-yl)-9-thiazol-4-yl-9H-purin-6-yl]-amine

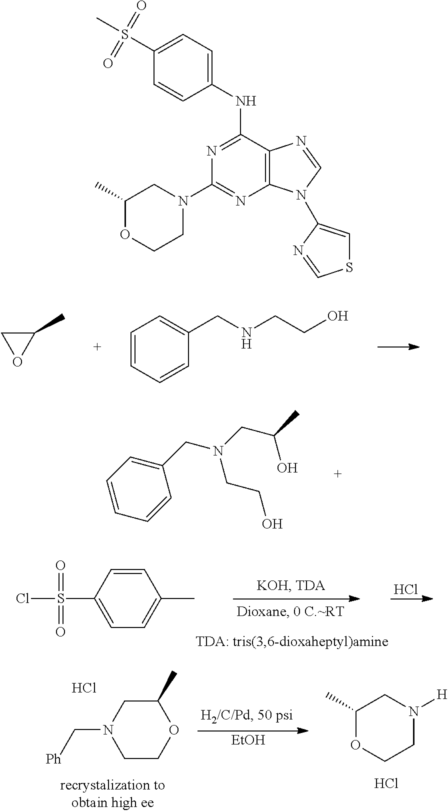

N-Benzylethanolamine (9.06 g, 60 mmol) is stirred with (R)-(+)-propylene oxide (6.96 g, 99%, 120 mmol) in a sealed tube at 45° C. overnight. Evaporation of the excess of propylene oxide in vacuo gives the diol residue which is used directly for the next step.

The diol is dissolved in dioxane (60 mL, anhydrous). KOH (10.08 g, 180 mmol) and tris(3,6-dioxaheptyl)amine (200 mg, 0.62 mmol) are added and the mixture is cooled to 0° C. after which tosyl chloride (12.58 g, 66 mmol, in 60 mL anhydrous dioxane) is added dropwise. The reaction mixture is allowed to stir at 0° C. for 45 minutes after which it is warmed to room temperature and stirred for an additional 4 hours. The reaction mixture is filtered and the filtrate is evaporated in vacuo. HCl (2 N, 200 mL) is added to the product and the resulting acidic aqueous solution is washed with ethyl acetate (150 mL×2), the solution cooled to 0° C. and neutralized by adding NaOH. The product is then extracted with ethyl acetate. The organic phase is dried with Na₂SO₄ and then subjected to evaporation. The residue is chromatographed (5˜20% ethyl acetate in DCM) to give the cyclized product (6.66 g).

The free base is converted to the HCl salt and recrystallized as follows: The free base obtained above is treated with HCl (2 M in ether, 50 mL) and subject to evaporation to yield the HCl salt. The salt (6.0 gram) is mixed with ethyl acetate (120 mL) and heated to reflux. EtOH is added dropwise cautiously until the entire solid has dissolved. Then it is cooled to room temperature and kept in the refrigerator overnight. The precipitate obtained is filtered to give pure product (2.8 g).

A solution of the recrystallized salt (1.35 g, 5.94 mmol) in ethanol (30 mL) is hydrogenated over 10% Pd/C (0.20 g) under pressure (55 psi) at room temperature overnight. The mixture is filtered through celite (washed with EtOH) and the filtrate is evaporated to give oil. Addition of ether and subsequent evaporation gives R-2-methylmorpholine hydrochloride as solid.

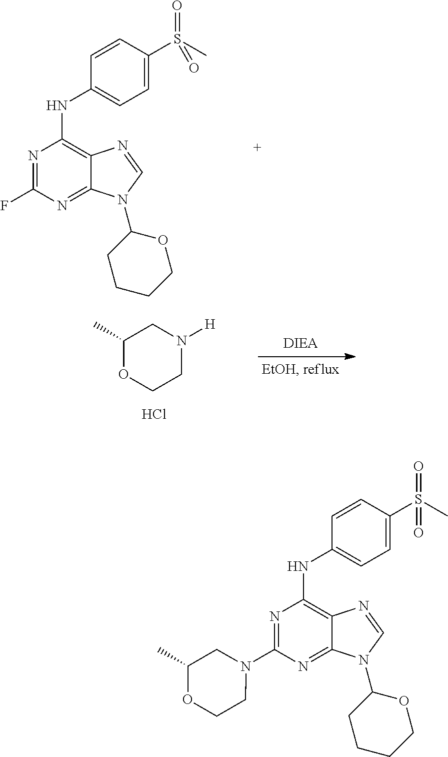

The mixture of the 2-fluoropurine substrate (4.6 g, 11.8 mmol), R-2-methylmorpholine hydrochloride (1.78 g, 12.9 mmol) and DIEA (3.78 g, 29.4 mmol) in ethanol (20 ml) is refluxed overnight. Ethanol is evaporated and the residue is redissolved in DCM (100 ml). It is washed with saturated NaHCO₃ (50 ml), water (50 ml), brine (50 ml) and dried over MgSO₄. Evaporation of the solvent followed by column chromatography (EtOAc/DCM from 30% to 50%) yields R-4-methanesulfonyl-phenyl)-[2-(2-methyl-morpholin-4-yl)-9-(tetrahydro-pyran-2-yl)-9H-purin-6-yl]-amine as pale brown solid.

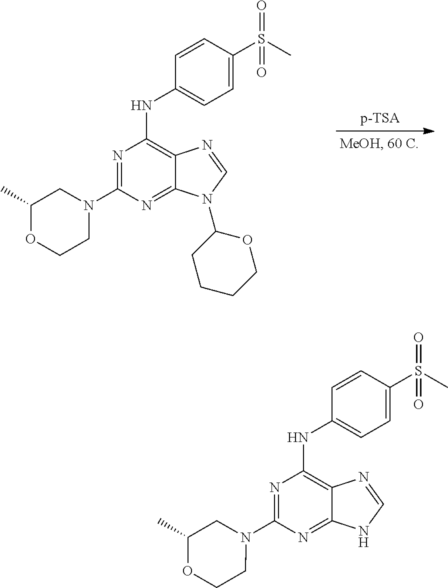

The compound obtained above (1.90 g, 4.02 mmol) is stirred with p-toluenesulfonic acid monohydrate (380 mg, 2.0 mmol) in methanol (20 mL) at 60° C. until the starting material is no longer detected (monitored by TLC or LC-MS). Triethylamine (0.5 mL) is added and ethanol is evaporated. Column chromatography (MeOH/DCM from 0 to 5%) yields the deprotection product.

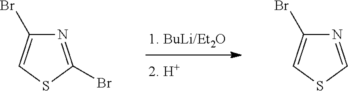

2,4-Dibromothiazole (5.00 g, 20.7 mmol) is placed in a flask which has been back filled with Argon three times. Anhydrous ether (82 mL) is added and the solution is cooled to −78° C. n-Butyllithium (2.5 M in cyclohexane, 10.0 mL) is added and the reaction mixture is stirred for 90 minutes at −78° C. before quenching with HCl/ether solution (2.0 m×15 mL). The reaction mixture is warmed to room temperature. The mixture is washed with NaHCO₃ (saturated aqueous solution, 60 mL) and the organic phase is dried with Na₂SO₄. After evaporation, 4-bromothiazole is obtained as a crude product.

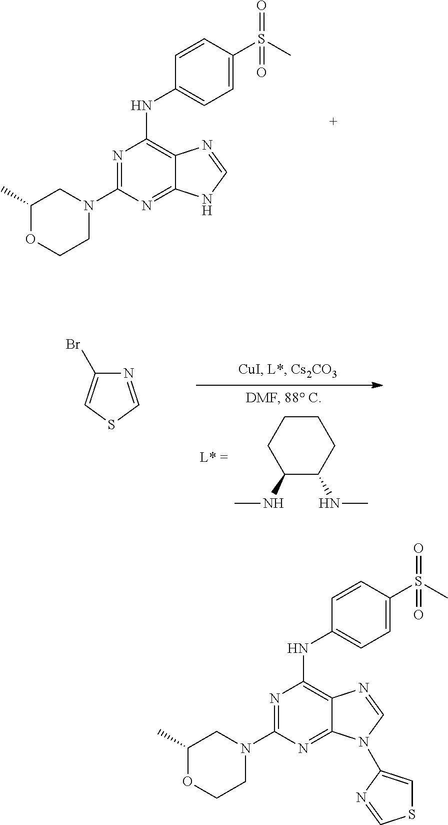

The deprotected 2,6-disubstituted purine (1.44 g, 3.71 mmol), CuI (352 mg, 1.86 mmol) and Cs₂CO₃ (3.62 g, 3.0 eq) are combined in a flask (previously backfilled with argon). Trans-N,N′-dimethylcyclohexane-1,2-diamine (264 mg, 1.86 mmol) and 4-bromothiazole (691 mg, 88% pure, 3.71 mmol) in DMF (8.0 mL) is added and the mixture is stirred at 88° C., overnight. After the mixture is cooled to room temperature, acetic acid (1.0 mL) is added and the mixture is filtered through a syringe filter (washed with DMF). The filtrate purified by reverse-phase preparative LC-MS (acetonitrile/water/TFA gradient 10-90% CH3CN in 7.5 minutes, Ultro 120 5 uM C18Q, 75×30 mmID). The collected water/MeCN solution of the product is evaporated to remove the acetonitrile. NaHCO₃ (saturated aqueous solution) is added to raise the pH to 9. DCM is used to extract the product and the organic phase is dried with Na₂SO₄. Evaporation of the solvent yields R-(4-Methanesulfonyl-phenyl)-[2-(2-methyl-morpholin-4-yl)-9-thiazol-4-yl-9H-purin-6-yl]-amine as free base/white powder; ¹H NMR 400 MHz (CDCl₃) δ 9.69 (s, 1H), 8.87 (d, 1H, J=2.4 Hz), 8.83 (s, 1H), 8.26 (d, 1H, J=2.4 Hz), 8.07 (d, 2H, J=8.8 Hz), 7.95 (d, 2H, J=8.8 Hz), 4.53 (t, 2H, J=10.8 Hz), 4.10-4.07 (m, 1H), 3.74-3.65 (m, 2H), 3.25-3.10 (m, 1H), 3.08 (s, 3H), 2.90-2.84 (m, 1H), 1.33 (d, 3H, J=6.4 Hz); MS m/z 472.3 (M+1).

### Example 12

### 1-(4-{2-[Methyl-(1-methyl-piperidin-4-yl)-amino]-9-thiazol-4-yl-9H-purin-6-ylamino}-phenyl)-ethanone

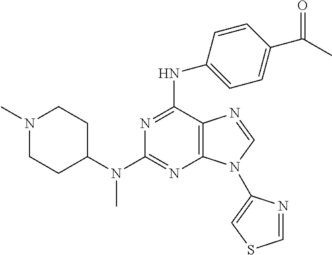

1-(4-Amino-phenyl)-ethanone (1.0 g, 7.4 mmol) is mixed with 2-fluoro-6-chloro-9-(tetrahydro-pyran-2-yl)-9H-purine (1.90 g, 7.4 mmol), diisopropylethylamine (1.54 mL, 8.9 mmol) and n-butanol 50 mL. The reaction is stirred in 95° C. for 14 hours. After cooling down to the room temperature and removing the solvent, the crude product is purified by flash chromatography using MeOH/DCM (5%:95%) to get 1-{4-[2-Fluoro-9-(tetrahydro-pyran-2-yl)-9H-purin-6-ylamino]-phenyl}-ethanone white solid 2.49 g.

1-{4-[2-Fluoro-9-(tetrahydro-pyran-2-yl)-9H-purin-6-ylamino]-phenyl}-ethanone (100 mg, 0.28 mmol) is mixed with methyl-(1-methyl-piperidin-4-yl)-amine (58 mg, 0.45 mmol), diisopropylethylamine (121 μL, 0.70 mmol) and 5 mL n-butanol. The reaction is stirred in 100° C. for 14 hours. After cooling down and remove the solvent, the crude product is purified by flash chromatography using EA/Hexane (1:1) to get 1-{4-[2-[Methyl-(1-methyl-piperidin-4-yl)-amino]-9-(tetrahydro-pyran-2-yl)-9H-purin-6-ylamino]-phenyl}-ethanone yellow solid 115 mg.

1-{4-[2-[Methyl-(1-methyl-piperidin-4-yl)-amino]-9-(tetrahydro-pyran-2-yl)-9H-purin-6-ylamino]-phenyl}-ethanone (115 mg, 0.26 mmol) is dissolved in 10 mL ethanol and mixed with 200 μL TFA. The reaction is stirred in 60° C. for 2 hours. After cooling down to the room temperature and totally removing the solvent and TFA, the crude product is mixed with copper (I) iodide (50 mg, 0.26 mmol) and potassium phosphate (220 mg, 0.8 mmol) and degassed and refilled with dry nitrogen. N,N′-Dimethylethylenediamine (46 mg, 0.52 mmol) and iodo-thiazole (53 mg, 0.26 mmol) in DMF (4 mL) are added and the mixture is stirred at 90° C. for 14 hours. After cooling down to room temperature, AcOH-MeOH (1:10, 1.6 mL) is added to neutralize the mixture followed by filtration through a syringe filter. After removing the solvent, the crude product is dissolved in DMSO and purified by preparative HPLC to get a pale solid 1-(4-{2-[Methyl-(1-methyl-piperidin-4-yl)-amino]-9-thiazol-4-yl-9H-purin-6-ylamino}-phenyl)-ethanone: ¹H NMR 400 MHz (DMSO-d₆) δ 10.22 (s, 1H), 9.28 (d, 1H, J=2.3), 8.61 (s, 1H), 8.25 (d, 1H, J=2.1 Hz), 8.12 (d, 2H, J=8.7 Hz), 7.98 (d, 2H, J=8.7 Hz), 3.57 (m, 4H), 3.21 (t, 1H, J=4.6 Hz), 3.10 (s, 3H), 2.79 (d, 3H, J=4.6 Hz), 2.55 (s, 3H), 2.00 (m, 4H) (MS m/z 463.3 (M+1).

By repeating the procedures described in the above examples, using appropriate starting materials, the following compounds of Formula I, as identified in Tables 1, 2 and 3, are obtained.

TABLE 1

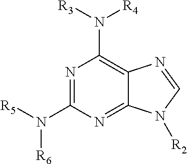

| | | | | | | Phys- |
|---|---|---|---|---|---|---|
| | | | | | | ical |
| Com- | | | | | | Data |
| pound | | | | | | MS |
| Num- | | | | | | (m/z): |
| ber | R₆ | R₅ | R₄ | R₃ | R₂ | M + 1 |
| 10 | 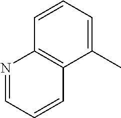 | H | 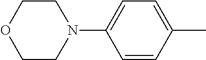 | H | 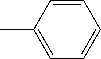 | 515.3 |
| 11 | 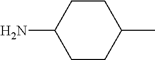 | H | 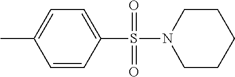 | H | 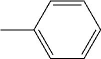 | 547.2 |
| 12 | 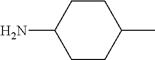 | H | 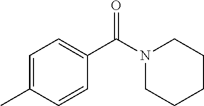 | H | 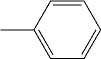 | 511.3 |

Additional Physical Data for Compound 12
¹H NMR 400 MHz (CD₃OD) d 8.03 (s, 1H), 7.90-7.95 (m, 2H), 7.75-7.65 (m, 2H), 7.50-7.42 (m, 2H), 7.38-7.30 (m, 3H), 3.80-3.50 (m, 5H),
2.83-2.73 (m, 1H), 2.15-2.05 (m, 2H), 1.95-1.90 (m, 2H), 1.70-1.40 (m, 6H), 1.40-1.20 (m, 4H)

| | | | | | | |
|---|---|---|---|---|---|---|
| 13 | 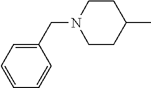 | H | 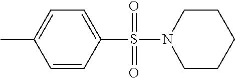 | H | 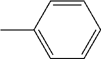 | 623.2 |
| 14 | 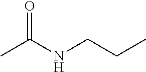 | H | 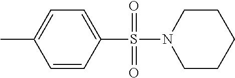 | H | 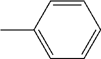 | 535.2 |
| 15 | 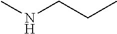 | CH₃ | 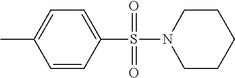 | H | 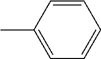 | 521.2 |
| 16 | 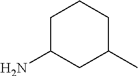 | H | 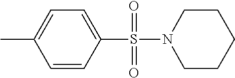 | H | 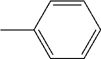 | 547.2 |
| 17 | 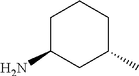 | H | 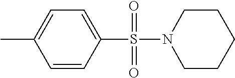 | H | 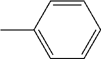 | 547.2 |
| 18 | 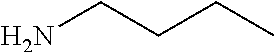 | CH₃ | 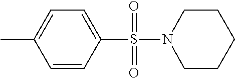 | H | 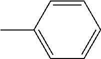 | 521.2 |
| 19 | 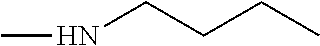 | CH₃ | 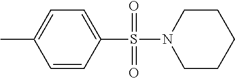 | H | 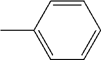 | 535.2 |
| 20 | 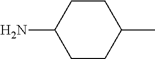 | H | 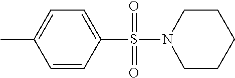 | H | 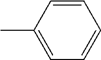 | 547.2 |
| 21 | 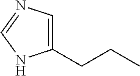 | H | 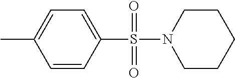 | H | 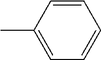 | 545.2 |
| 22 | 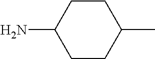 | H | 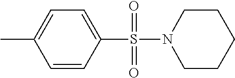 | H | 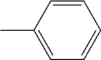 | 547.2 |
| 23 | 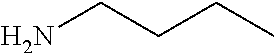 | H | 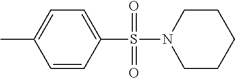 | H | 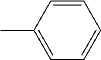 | 507.2 |
| 24 | 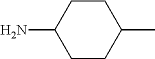 | H | 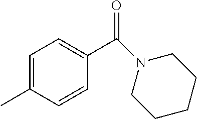 | H | H | 435.2 |
| 25 | 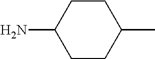 | H | 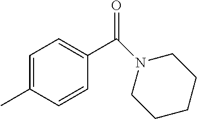 | H | 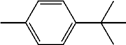 | 567.4 |
| 26 | 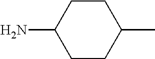 | H | 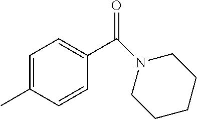 | H | 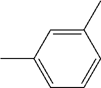 | 525.3 |
| 27 | 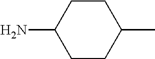 | H | 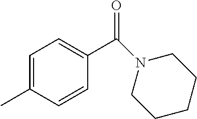 | H | 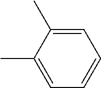 | 525.3 |
| 28 | 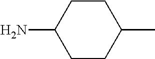 | H | 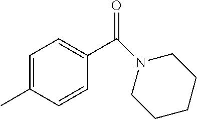 | H | 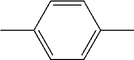 | 525.3 |
| 29 | 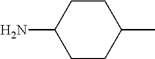 | H | 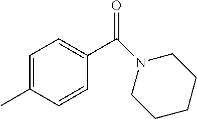 | H | 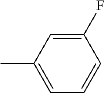 | 529.3 |
| 30 | 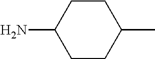 | H | 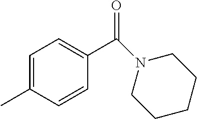 | H | 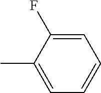 | 529.3 |
| 31 | 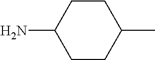 | H | 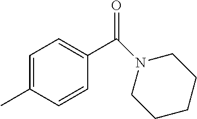 | H | 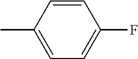 | 529.3 |
| 32 | 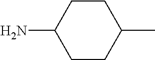 | H | 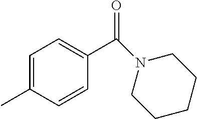 | H | 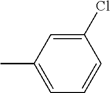 | 545.3 |
| 33 | 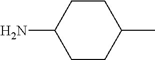 | H | 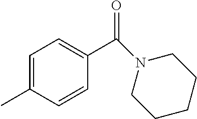 | H | 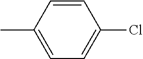 | 545.3 |
| 34 | 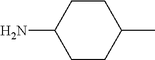 | H | 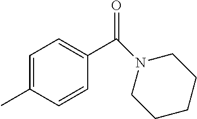 | H | 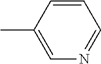 | 512.3 |
| 35 | 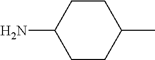 | H | 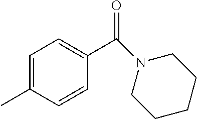 | H | 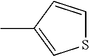 | 517.3 |

Additional Physical Data for Compound 35
¹H NMR 400 MHz (CD₃OD) d 8.16 (s, 1H), 8.02-7.90 (m, 3H), 7.70-7.62 (m, 1H), 7.60-7.55 (m, 1H), 7.40 (d, 2H, J = 8.4 Hz), 3.82-3.40
(m, 5H), 2.76-2.64 (m, 1H), 2.20-2.10 (m, 2H), 2.00-1.90 (m, 2H), 1.80-1.50 (m, 6H), 1.45-1.25 (m, 4H).

| | | | | | | |
|---|---|---|---|---|---|---|
| 36 | 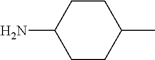 | H | 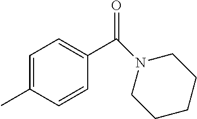 | H | 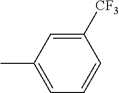 | 579.3 |
| 37 | 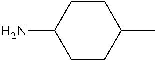 | H | 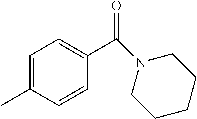 | H | 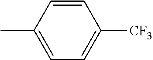 | 579.3 |
| 38 | 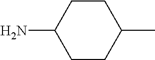 | H | 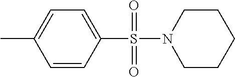 | H | 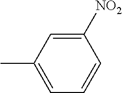 | 556.3 |
| 39 | (CH₂)₄N(CH₃)₂ | H | 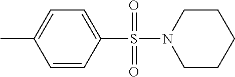 | H | 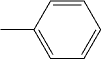 | 549.3 |
| 40 | (CH₂)₄NH₂ | H | 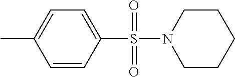 | H | 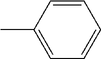 | 521.3 |
| 41 | (CH₂)₃N(CH₃)₂ | H | 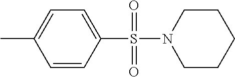 | H | 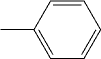 | 535.3 |
| 42 | (CH₂)CH(CH₃)NH₂ | H | 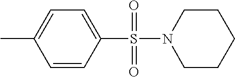 | H | 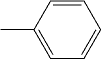 | 507.2 |
| 43 | (CH₂)₂NH₂ | H | 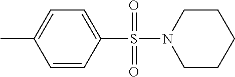 | H | 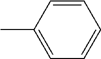 | 493.2 |
| 44 | (CH₂)₂OH | (CH₂)₂OH | 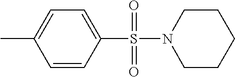 | H | 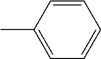 | 538.2 |
| 45 | (CH₂)₂OH | H | 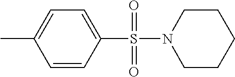 | H | 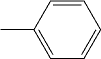 | 494.2 |
| 46 | (CH₂)₂OH | CH₃ | 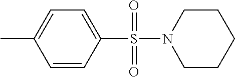 | H | 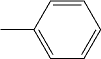 | 508.2 |
| 47 | (CH₂)₂OCH₃ | (CH₂)₂OCH₃ | 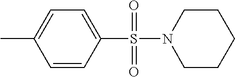 | H | 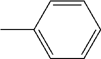 | 566.3 |
| 48 | CH(C₃H₇)CH₂OH | H | 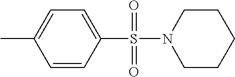 | H | 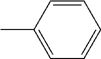 | 536.3 |
| 49 | 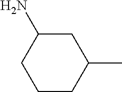 | H | 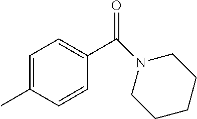 | H | 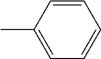 | 511.2 |
| 50 | (CH₂)₃NH₂ | CH₃ | 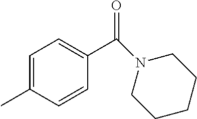 | H | 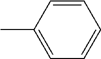 | 485.2 |
| 51 | (CH₂)₃NHCH₃ | CH₃ | 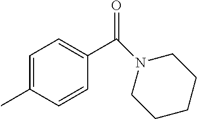 | H | 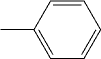 | 499.3 |
| 52 | 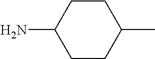 | H | 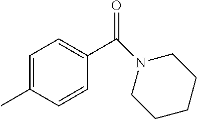 | H | 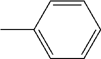 | 511.3 |
| 53 | (CH₂)₃NH₂ | H | 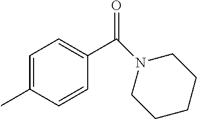 | H | 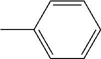 | 471.3 |
| 54 | 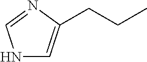 | H | 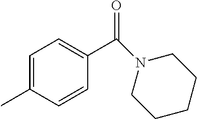 | H | 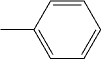 | 508.3 |
| 55 | 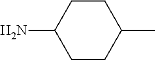 | H | 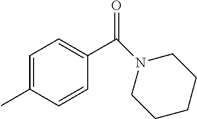 | H | 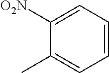 | 556.3 |
| 56 | 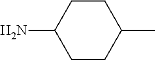 | H | 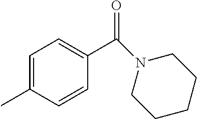 | H | 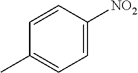 | 556.3 |
| 57 | 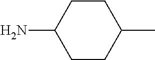 | H | 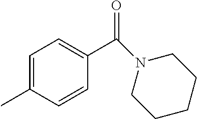 | H | 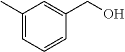 | 541.2 |
| 58 | 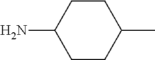 | H | 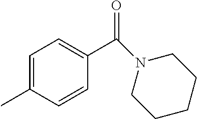 | H | 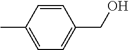 | 541.2 |
| 59 | 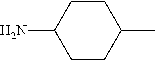 | H | 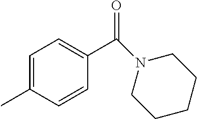 | H | 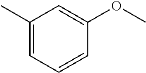 | 541.2 |
| 60 | 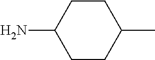 | H | 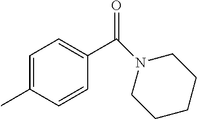 | H | 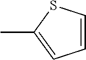 | 517.2 |
| 61 | 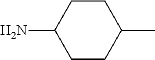 | H | 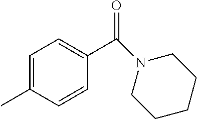 | H | 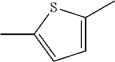 | 531.2 |
| 62 | 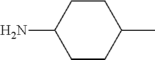 | H | 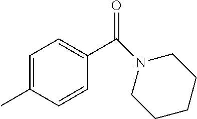 | H | 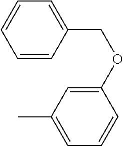 | 617.3 |
| 63 | 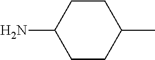 | H | 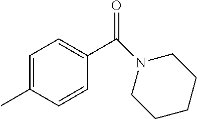 | H | 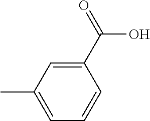 | 555.2 |
| 64 | 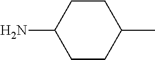 | H | 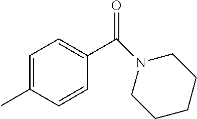 | H | 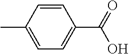 | 555.2 |
| 65 | 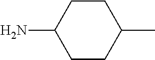 | H | 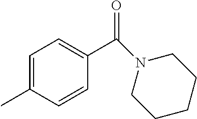 | H | 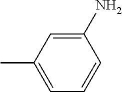 | 526.2 |
| 66 | 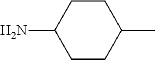 | H | 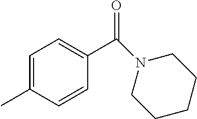 | H | 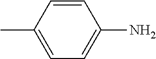 | 525.25 |
| 67 | 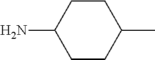 | H | 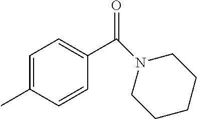 | H | 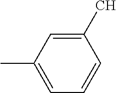 | 536.25 |
| 68 | 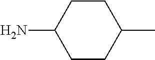 | H | 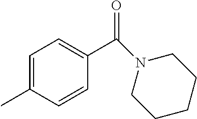 | H | 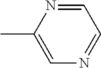 | 513.20 |
| 69 | 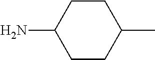 | H | 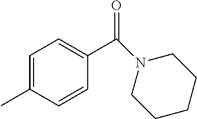 | H | 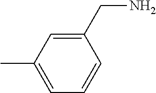 | 540.30 |
| 70 | 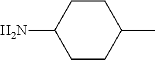 | H | 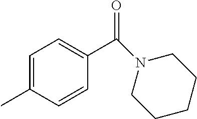 | H | 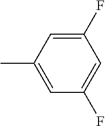 | 547.20 |
| 71 | 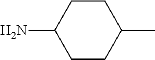 | H | 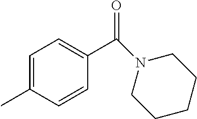 | H | 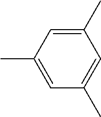 | 539.30 |
| 72 | 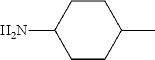 | H | 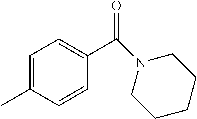 | H | 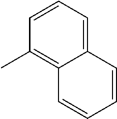 | 561.25 |
| 73 | 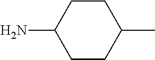 | H | 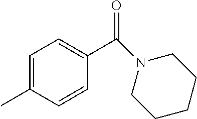 | H | 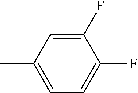 | 547.20 |
| 74 | 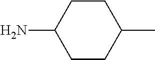 | H | 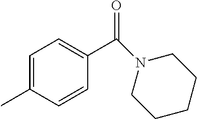 | H | 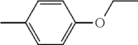 | 555.30 |
| 75 | 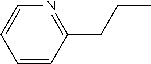 | CH₃ | 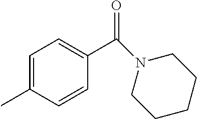 | H | 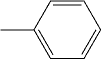 | 533.3 |
| 76 | 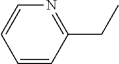 | H | 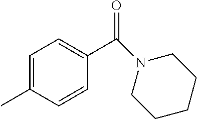 | H | 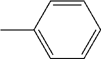 | 505.3 |
| 77 | 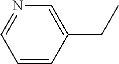 | H | 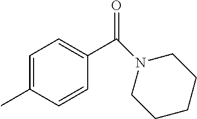 | H | 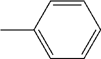 | 505.3 |
| 78 | 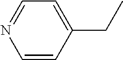 | H | 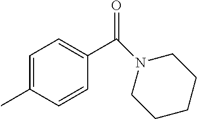 | H | 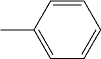 | 505.3 |
| 79 | 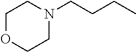 | H | 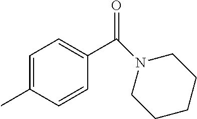 | H | 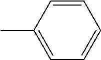 | 541.3 |
| 80 | 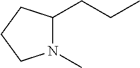 | H | 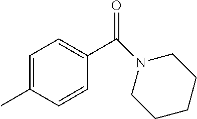 | H | 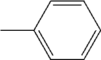 | 525.4 |
| 81 | 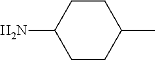 | H | 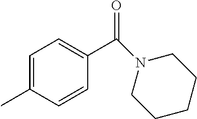 | H | 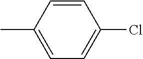 | 546.2 |
| 82 | 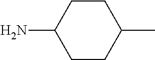 | H | 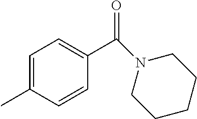 | H | 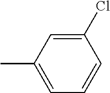 | 546.2 |
| 83 | 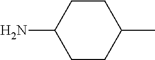 | H | 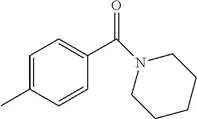 | H | 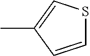 | 517.3 |
| 84 | 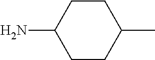 | H | 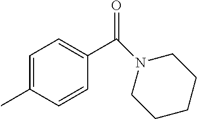 | H | 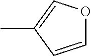 | 501.30 |
| 85 | 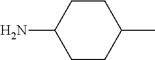 | H | 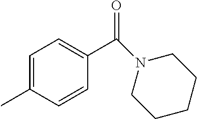 | H | 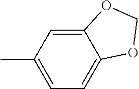 | 555.3 |
| 86 | 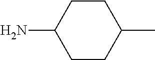 | H | 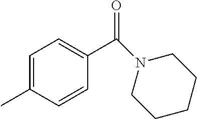 | H | 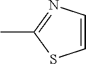 | 518.3 |
| 87 | 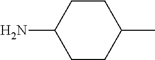 | H | 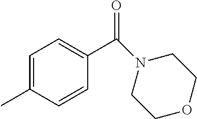 | H | 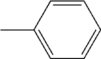 | 513.20 |
| 88 | 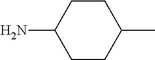 | H | 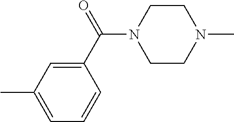 | H | 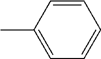 | 526.25 |
| 89 | 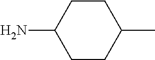 | H | 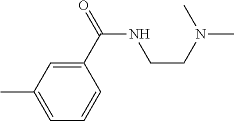 | H | 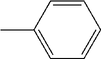 | 514.20 |
| 90 | 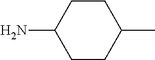 | H | 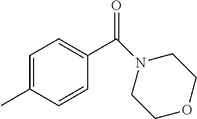 | H | 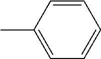 | 513.20 |
| 91 | 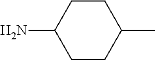 | H | 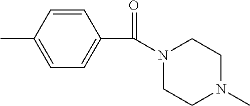 | H | 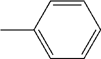 | 526.30 |
| 92 | 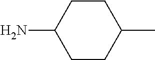 | H | 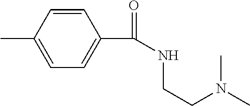 | H | 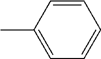 | 513.20 |
| 93 | 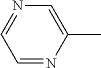 | H | 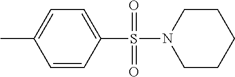 | H | 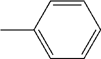 | 528.25 |
| 94 | 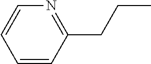 | H | 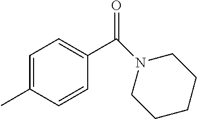 | H | 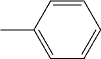 | 519.3 |
| 95 | 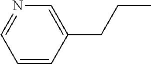 | H | 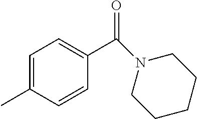 | H | 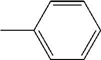 | 519.3 |
| 96 | 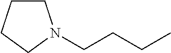 | H | 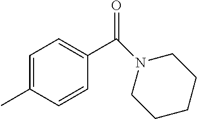 | H | 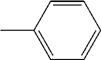 | 525.35 |
| 97 | 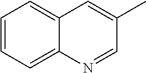 | H | 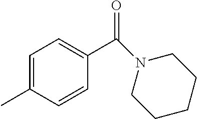 | H | 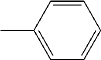 | 541.3 |
| 98 | 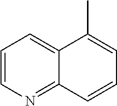 | H | 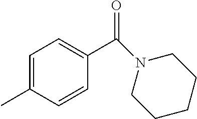 | H | 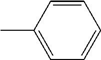 | 541.3 |
| 99 | 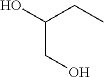 | H | 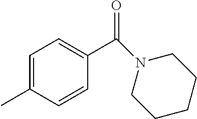 | H | 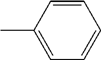 | 488.3 |
| 100 | 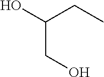 | CH₃ | 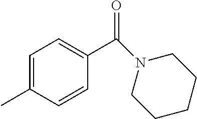 | H | 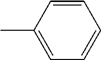 | 502.3 |
| 101 | 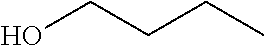 | H | 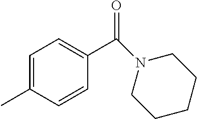 | H | 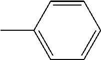 | 472.3 |
| 102 | 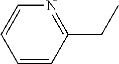 | H | 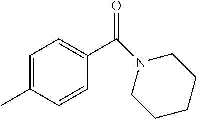 | H | 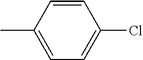 | 540.30 |
| 103 | 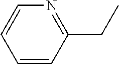 | H | 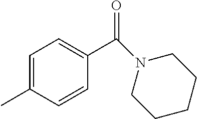 | H | 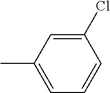 | 540.30 |
| 104 | 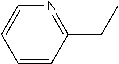 | H | 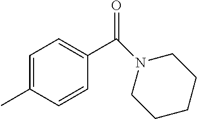 | H | 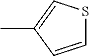 | 511.3 |
| 105 | 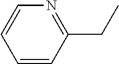 | H | 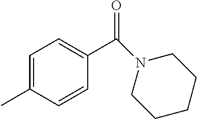 | H | 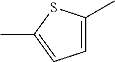 | 525.3 |
| 106 | 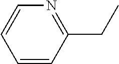 | H | 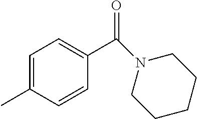 | H | 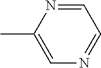 | 507.30 |
| 107 | 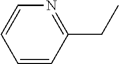 | H | 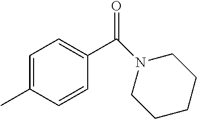 | H | 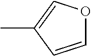 | 495.3 |
| 108 | 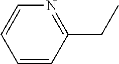 | H | 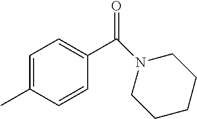 | H | 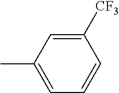 | 573.3 |
| 109 | 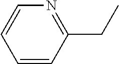 | H | 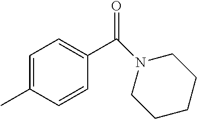 | H | 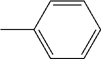 | 505.3 |
| 110 | 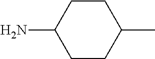 | H | 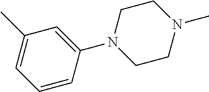 | H | 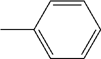 | 498.3 |

Additional Physical Data for Compound 110
¹H NMR 400 MHz (DMSO-d₆) δ 9.12 (s, 1H), 8.16 (s, 1H), 7.78 (d, 2H), 7.58 (d, 1H), 7.42 (m, 2H), 7.24 (m, 2H), 7.00 (t, 1H), 6.48 (m, 2H),
3.53 (s, 1H), 3.25 (m, 4H), 3.01 (t, 4H), 2.09 (s, 3H), 1.74 (m, 2H), 1.66 (s, 2H), 0.92 (m, 4H), 0.79 (t, 1H); MS m/z 498.3 (M + 1)

| | | | | | | |
|---|---|---|---|---|---|---|
| 111 | 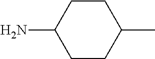 | H | 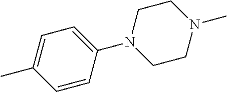 | H | 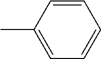 | 498.3 |
| 112 | 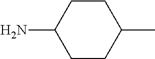 | H | 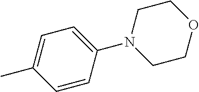 | H | 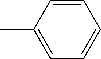 | 485.3 |

Additional Physical Data for Compound 112
¹H NMR 400 MHz (DMSO-d₆) δ 9.29 (s, 1H), 8.23 (s, 1H), 7.84 (t, 4H), 7.51 (t, 2H), 7.35 (t, 1H), 6.84 (d, 2H), 6.48 (d, 1H), 3.71 (t, 4H),
3.57 (s, 1H), 3.01 (t, 4H), 1.93 (d, 2H), 1.77 (d, 2H), 1.24 (m, 4H), 0.90 (t, 1H); MS m/z 485.3 (M + 1).

| | | | | | | |
|---|---|---|---|---|---|---|
| 113 | 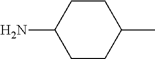 | H | 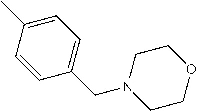 | H | 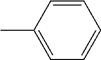 | 499.2 |
| 114 | 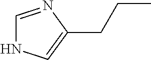 | H | 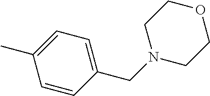 | H | 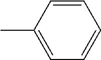 | 496.3 |
| 115 | 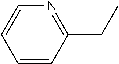 | H | 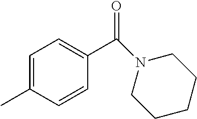 | H | 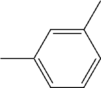 | 519.40 |
| 116 | 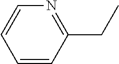 | H | 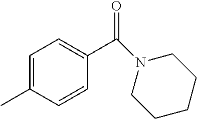 | H | 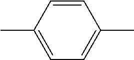 | 519.30 |
| 117 | 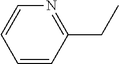 | H | 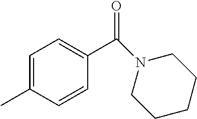 | H | 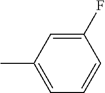 | 523.30 |
| 118 | 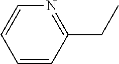 | H | 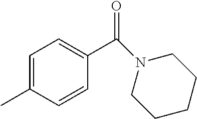 | H | 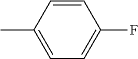 | 523.30 |
| 119 | 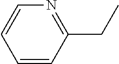 | H | 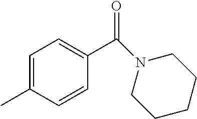 | H | 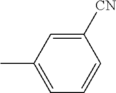 | 530.30 |
| 120 | 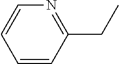 | H | 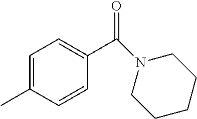 | H | 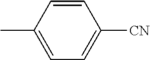 | 530.30 |
| 121 | 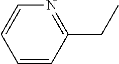 | H | 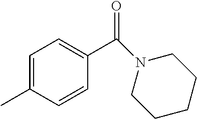 | H | 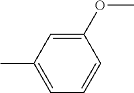 | 535.30 |
| 122 | 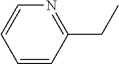 | H | 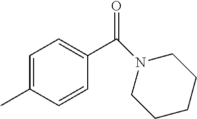 | H | 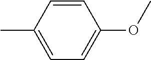 | 535.30 |
| 123 | 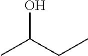 | H | 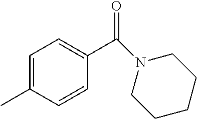 | H | 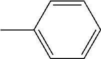 | 472.3 |

Additional Physical Data for Compound 123
¹H NMR 400 MHz (MeOH-d₄) δ 8.06 (s, 1H), 7.86 (d, 2H), 7.67 (d, 2H), 7.44 (t, 2H), 7.34 (d, 2H), 7.30 (d, 2H), 3.87-3.95 (m, 1H),
3.34-3.44 (m, 4H), 3.21-3.23 (m, 2H), 1.45-1.69 (m, 6H), 1.09 (d, 3H).

| | | | | | | |
|---|---|---|---|---|---|---|
| 124 | 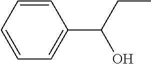 | H | 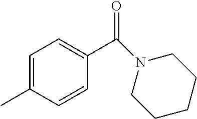 | H | 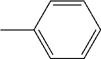 | 548.3 |
| 125 | 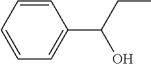 | H | 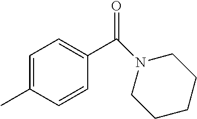 | H | 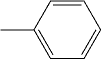 | 548.3 |
| 126 | 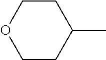 | H | 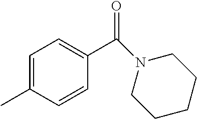 | H | 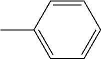 | 498.3 |
| 127 | 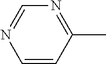 | H | 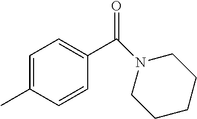 | H | 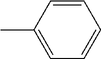 | 492.3 |
| 128 | 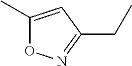 | H | 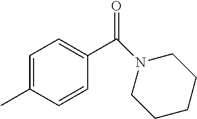 | H | 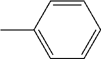 | 509.3 |
| 129 | 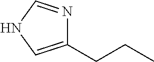 | H | 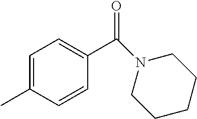 | H | 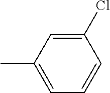 | 543.3 |
| 130 | 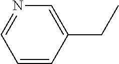 | H | 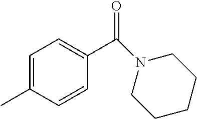 | H | 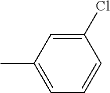 | 540.3 |
| 131 | 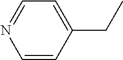 | H | 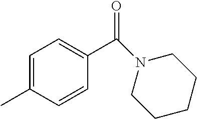 | H | 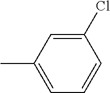 | 540.3 |

Additional Physical Data for Compound 131
¹H NMR 400 MHz (MeOH-d₄) δ 8.73 (d, 2H), 8.25 (s, 1H), 8.07 (d, 2H), 8.03-7.74 (m, 3H), 7.70-7.60 (m, 1H), 7.57-7.49 (m, 1H),
7.45-7.28 (m, 3H), 4.79 (s, 2H), 3.80-3.38 (m, 4H), 1.79-1.52 (m, 6H).

| | | | | | | |
|---|---|---|---|---|---|---|
| 132 | 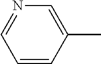 | H | 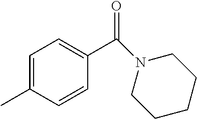 | H | 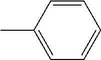 | 491.3 |
| 133 | 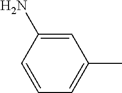 | H | 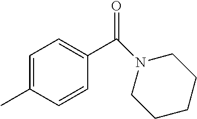 | H | 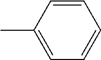 | 505.3 |

Additional Physical Data for Compound 133
¹H NMR 400 MHz (MeOH-d₄) δ 8.30 (s, 1H), 7.96 (d, 2H), 7.89 (t, 1H), 7.87 (d, 2H), 7.78 (d, 1H), 7.64 (t, 2H), 7.61 (t, 1H), 7.44 (d, 2H),
7.36 (t, 1H), 6.90 (d, 1H), 3.48-3.75 (m, 4H), 1.45-1.78 (m, 6H)

| | | | | | | |
|---|---|---|---|---|---|---|
| 134 | 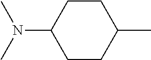 | H | 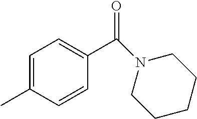 | H | 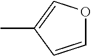 | 529.4 |
| 135 | 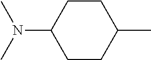 | H | 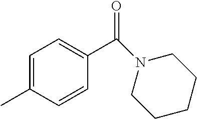 | H | 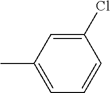 | 573.4 |
| 136 | 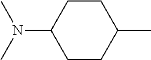 | H | 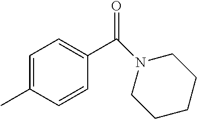 | H | 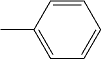 | 539.4 |
| 137 | 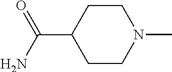 | H | 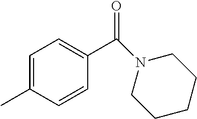 | H | 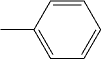 | 525.3 |
| 138 | 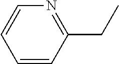 | H | 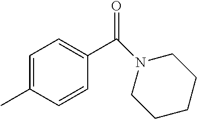 | H | 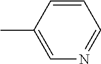 | 506.3 |
| 139 | 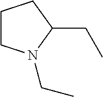 | H | 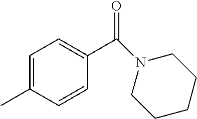 | H | 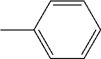 | 525.3 |
| 140 | 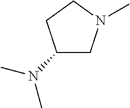 | H | 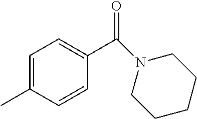 | H | 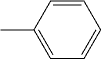 | 511.3 |
| 141 | 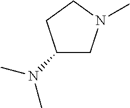 | H | 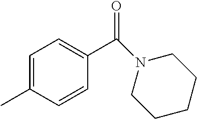 | H | 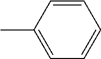 | 511.3 |

Additional Physical Data for Compound 141
¹H NMR 400 MHz (MeOH-d₄) δ 8.22 (s, 1H), 7.95 (d, 2H), 7.83 (d, 2H), 7.53 (t, 2H), 7.43 (d, 1H), 7.40 (d, 2H), 4.04-3.96 (m, 1H),
3.94-3.83 (m, 2H), 3.70-3.36 (m, 6H), 2.95 (s, 6H), 2.51-2.46 (m, 1H), 2.25-2.19 (m, 1H), 1.78-1.47 (m, 6H).

| | | | | | | |
|---|---|---|---|---|---|---|
| 142 | 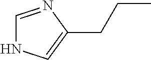 | H | 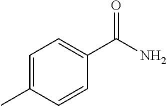 | H | 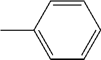 | 440.20 |
| 143 | 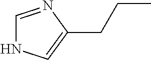 | H | 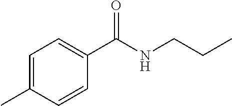 | H | 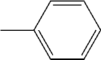 | 482.20 |
| 144 | 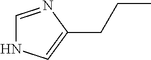 | H | 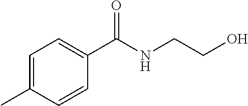 | H | 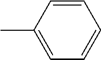 | 484.20 |
| 145 | 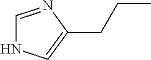 | H | 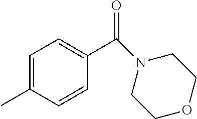 | H | 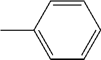 | 510.20 |
| 146 | 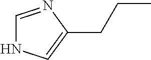 | H | 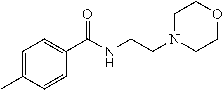 | H | 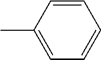 | 553.30 |
| 147 | 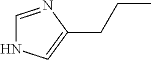 | H | 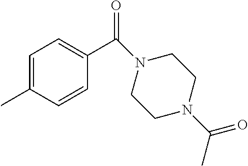 | H | 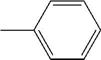 | 551.30 |
| 148 | 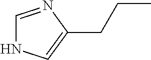 | H | 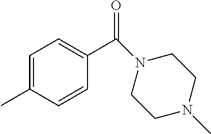 | H | 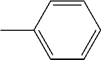 | 523.20 |
| 149 | 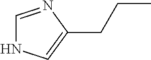 | H | 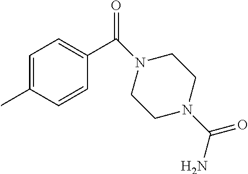 | H | 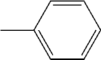 | 552.25 |
| 150 | 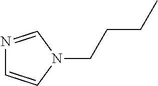 | H | 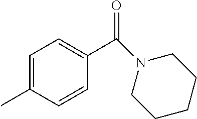 | H | 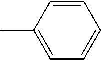 | 522.3 |

Physical Data for Compound 150
¹HNMR 400 MHz (MeOH-d₄) δ 8.86 (s, 1H), 8.31 (s, 1H), 7.86 (d, 2H), 7.75 (d, 2H), 7.61 (d, 1H), 7.58 (d, 2H), 7.52 (d, 1H), 7.45-7.43
(m, 3H), 4.32 (t, 2H), 3.71-3.63 (m, 2H), 3.56-3.47 (m, 4H), 2.23 (q, 2H), 1.79-1.47 (m, 6H).

| | | | | | | |
|---|---|---|---|---|---|---|
| 151 | 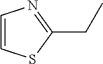 | H | 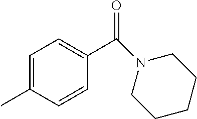 | H | 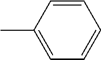 | 511.3 |
| 406 | 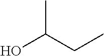 | H | 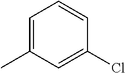 | H | 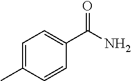 | 438.2 |
| 407 | 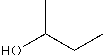 | H | 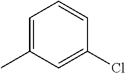 | H | 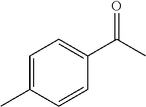 | 437.2 |
| 408 | 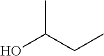 | H | 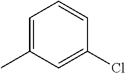 | H | 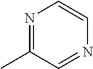 | 397.2 |
| 430 | 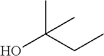 | H | 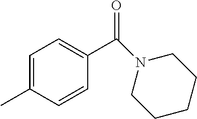 | H | 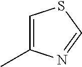 | 493.2 |
| 431 | 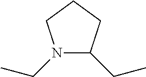 | H | 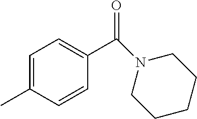 | H | 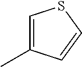 | 531.3 |
| 432 | 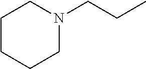 | H | 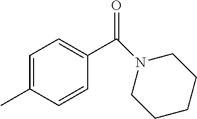 | H | 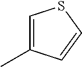 | 531.3 |
| 433 | 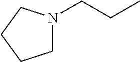 | H | 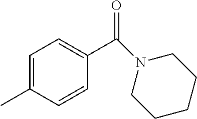 | H | 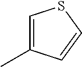 | 517.3 |
| 434 | 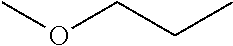 | H | 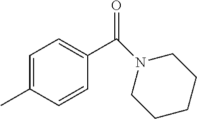 | H | 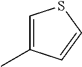 | 478.2 |
| 435 | 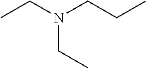 | H | 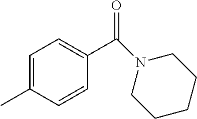 | H | 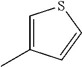 | 519.3 |
| 436 | 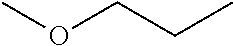 | H | 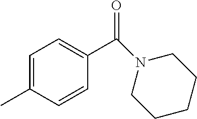 | H | 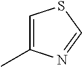 | 479.2 |
| 437 | 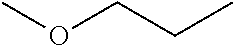 | H | 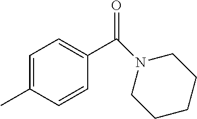 | H | 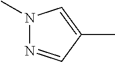 | 476.2 |
| 439 | 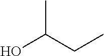 | H | 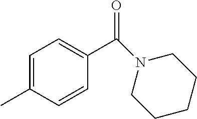 | H | 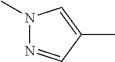 | 476.2 |
| 442 | 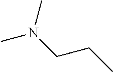 | H | 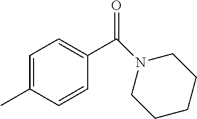 | H | 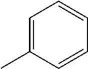 | 485.2 |
| 443 | 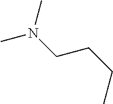 | H | 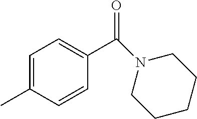 | H | 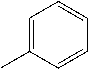 | 499.3 |
| 444 | 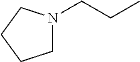 | H | 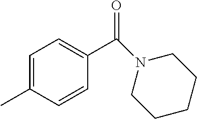 | H | 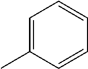 | 511.2 |
| 445 | 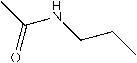 | H | 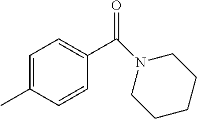 | H | 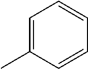 | 499.2 |
| 446 | 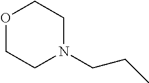 | H | 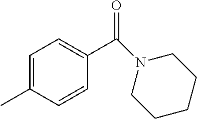 | H | 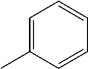 | 527.3 |
| 450 | 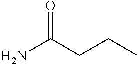 | H | 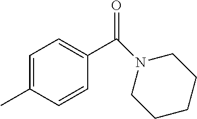 | H | 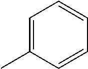 | 485.2 |
| 460 | 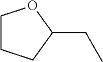 | H | 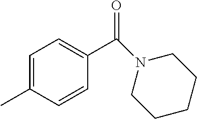 | H | 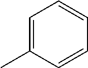 | 498.2 |
| 485 | C₄H₉— | H | 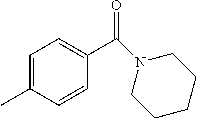 | H | 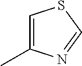 | 477.2 |
| 486 | 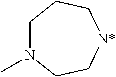 | H | 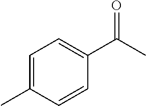 | H | 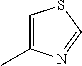 | 449.2 |

The components of Table 1 combine to form compounds of Formula I, for example, the components of compound 13 combine to form N²-(1-Benzyl-piperidin-4-yl)-9-phenyl-N6-[4-(piperidine-1-sulfonyl)-phenyl]-9H-purine-2,6-diamine, having the following structure:

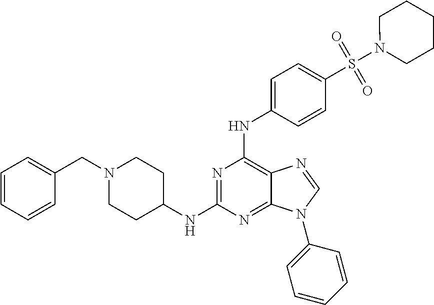

Similarly, the components of Table 2, combine to form compounds of Formula I. For example, the components of compound 425 combine to form (4-{2-[2-(4-methyl-thiazol-5-yl)-ethoxy]-9-thiophen-3-yl-9H-purin-6-ylamino}-phenyl)-piperidin-1-yl-methanone, having the following structure:

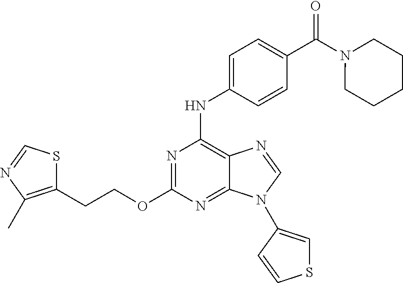

TABLE 2

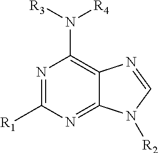

| | | | | | Physical |
|---|---|---|---|---|---|
| | | | | | Data |
| | | | | | MS |
| Compound | | | | | (m/z) |
| Number | R₁ | R₄ | R₃ | R₂ | M + 1 |
| 152 | Cl | 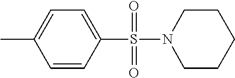 | H | 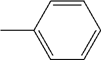 | 469.3 |
| 153 | CH₃O— | 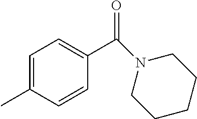 | H | 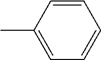 | 429.30 |
| 154 | H | 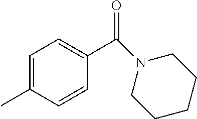 | H | 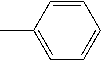 | 399.30 |
| 155 | H | 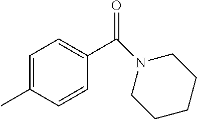 | H | 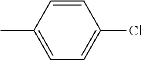 | 433.30 |
| 156 | H | 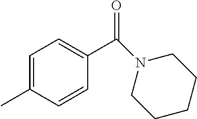 | H | 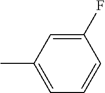 | 417.3 |
| 158 | H | 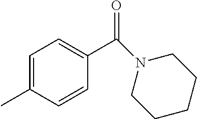 | H | 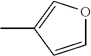 | 389.3 |
| 160 | H | 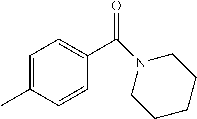 | H | 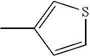 | 405.2 |
| 161 | H | 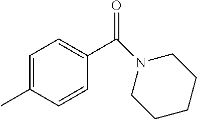 | H | 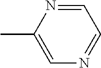 | 401.2 |
| 162 | H | 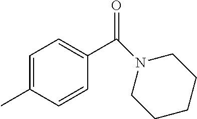 | H | 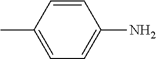 | 414.3 |
| 163 | H | 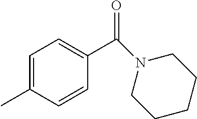 | H | 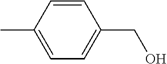 | 429.2 |
| 164 | H | 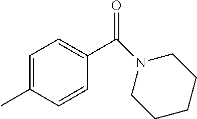 | H | 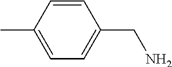 | 428.2 |
| 411 | 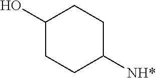 | 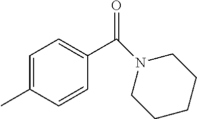 | H | 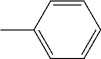 | 512.2 |
| 412 | 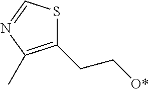 | 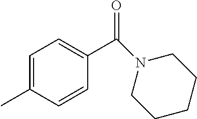 | H | 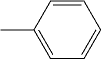 | 540.3 |
| 420 | H | 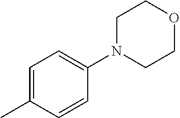 | H | 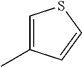 | 379.2 |
| 423 | CH₃O— | 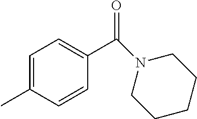 | H | 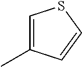 | 435.2 |
| 425 | 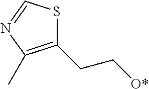 | 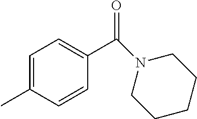 | H | 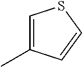 | 546.2 |
| 458 | 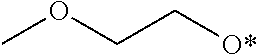 | 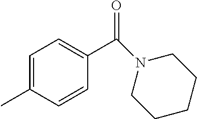 | H | 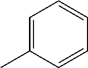 | 473.2 |
| 459 | 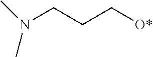 | 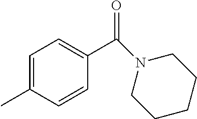 | H | 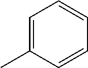 | 500.3 |
| 461 | 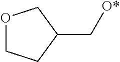 | 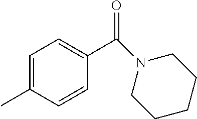 | H | 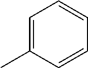 | 499.2 |
| 471 | 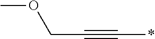 | 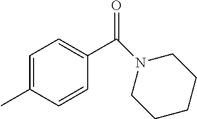 | H | 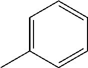 | 467.2 |
| 472 | 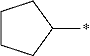 | 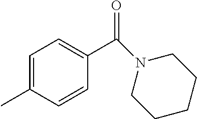 | H | 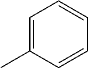 | 467.2 |
| 473 | 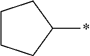 | 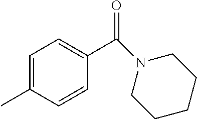 | H | 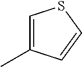 | 473.2 |
| 474 | 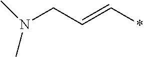 | 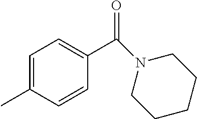 | H | 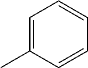 | 482.3 |
| 475 | 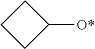 | 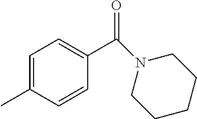 | H | 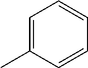 | 469.3 |
| 476 | 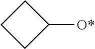 | 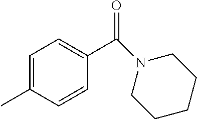 | H | 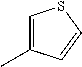 | 475.2 |
| 487 | 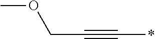 | 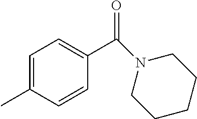 | H | 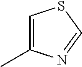 | 474.2 |
| 489 | 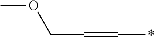 | 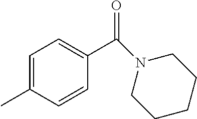 | H | 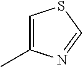 | 476.2 |
| 490 | 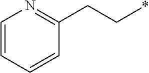 | 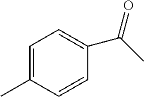 | H | 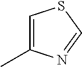 | 442.2 |

TABLE 3

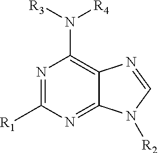

| | | | | | Physical Data |
|---|---|---|---|---|---|
| Compound | | | | | MS (m/z) |
| Number | R₁ | R₃ | R₄ | R₅ | M + 1 |
| 165 | 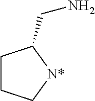 | 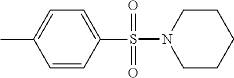 | H | 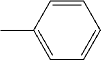 | 533.2 |
| 166 | 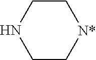 | 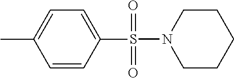 | H | 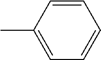 | 519.2 |
| 167 | 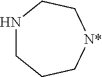 | 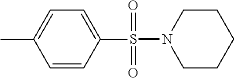 | H | 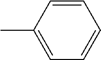 | 533.3 |
| 168 | 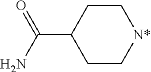 | 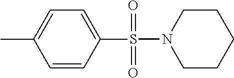 | H | 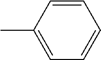 | 561.2 |
| 169 | 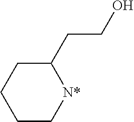 | 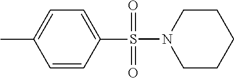 | H | 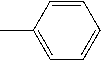 | 562.3 |
| 170 | 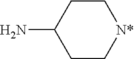 | 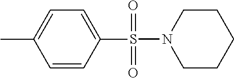 | H | 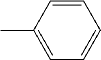 | 533.3 |
| 171 | 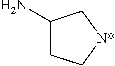 | 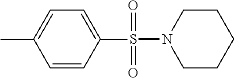 | H | 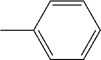 | 519.3 |
| 172 | 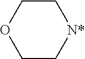 | 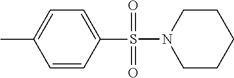 | H | 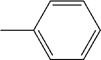 | 520.3 |
| 173 | 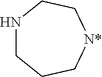 | 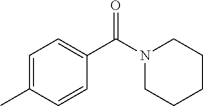 | H | 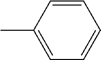 | 497.3 |
| 174 | 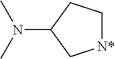 | 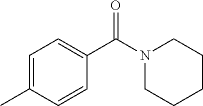 | H | 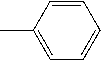 | 511.3 |
| 175 | 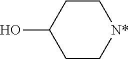 | 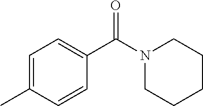 | H | 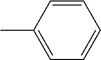 | 498.3 |
| 176 | 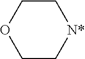 | 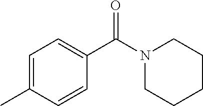 | H | 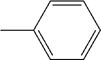 | 484.30 |
| 177 | 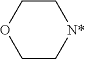 | 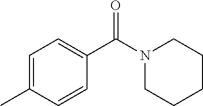 | H | 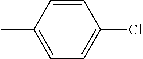 | 518.30 |
| 178 | 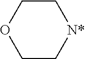 | 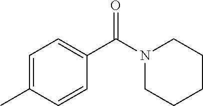 | H | 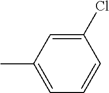 | 518.30 |
| 179 | 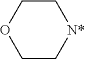 | 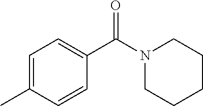 | H | 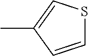 | 490.30 |
| 180 | 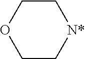 | 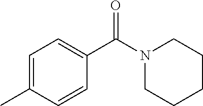 | H | 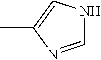 | 474.30 |
| 181 | 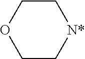 | 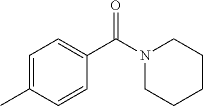 | H | 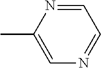 | 486.30 |
| 182 | 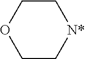 | 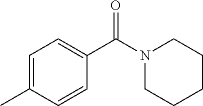 | H | 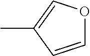 | 474.30 |
| 183 | 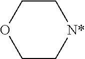 | 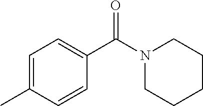 | H | 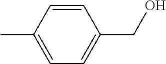 | 514.30 |
| 184 | 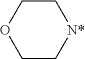 | 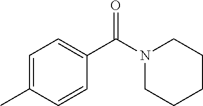 | H | 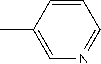 | 485.30 |
| 185 | 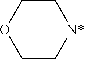 | 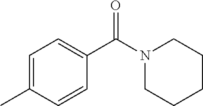 | H | 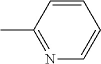 | 485.30 |
| 186 | 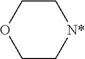 | 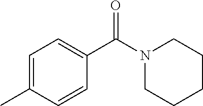 | H | 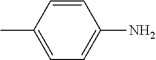 | 499.4 |
| 187 | 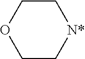 | 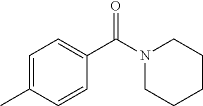 | H | 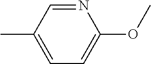 | 515.35 |
| 188 | 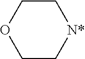 | 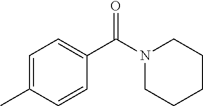 | H | 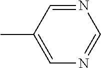 | 486.35 |
| 189 | 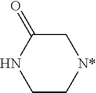 | 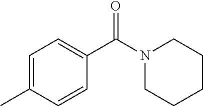 | H | 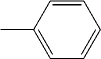 | 497.4 |

Additional Physical Data for Compound 189
¹H NMR 400 MHz (DMSO-d₆) δ 10.07 (s, 1H), 8.55 (s, 1H), 8.17 (s, 1H), 8.05 (d, 2H), 8.02 (d, 2H), 7.68 (t, 2H), 7.51 (t, 1H), 7.44 (d,
2H), 4.27 (s, 2H), 3.94-3.99 (m, 2H), 3.49-3.57 (m, 4H), 3.28-3.45 (m, 2H), 1.58-1.75 (m, 6H).

| | | | | | |
|---|---|---|---|---|---|
| 192 | 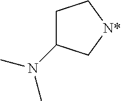 | 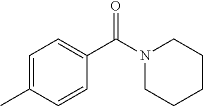 | H | 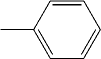 | |
| 193 | 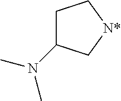 | 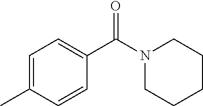 | H | 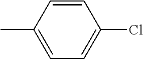 | 545.30 |
| 194 | 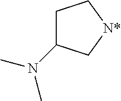 | 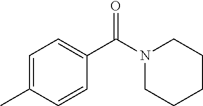 | H | 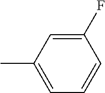 | 529.40 |
| 195 | 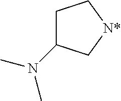 | 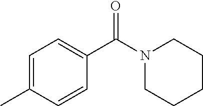 | H | 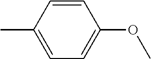 | 541.40 |
| 196 | 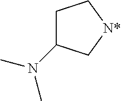 | 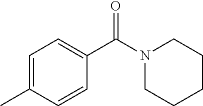 | H | 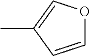 | 501.40 |
| 197 | 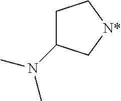 | 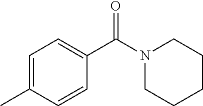 | H | 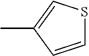 | 517.40 |
| 199 | 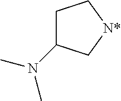 | 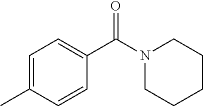 | H | 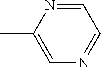 | 513.40 |
| 200 | 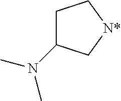 | 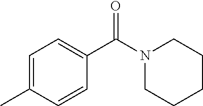 | H | 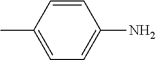 | 526.40 |
| 201 | 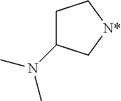 | 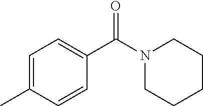 | H | 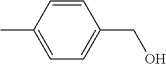 | 541.40 |
| 202 | 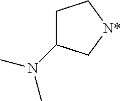 | 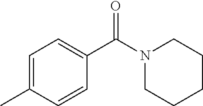 | H | 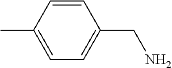 | 540.40 |
| 203 | 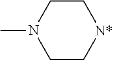 | 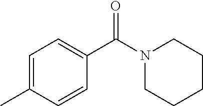 | H | 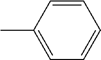 | 497.3 |
| 204 | 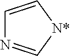 | 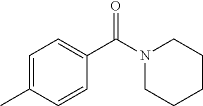 | H | 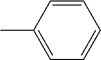 | 465.3 |

Additional Physical Data for Compound 204
¹H NMR 400 MHz (MeOH-d₄) δ 9.52 (s, 1H), 8.58 (s, 1H), 8.26 (m, 1H), 7.91 (d, 2H), 7.86 (d, 2H), 7.65 (m, 3H), 7.56 (d, 1H), 7.51 (d,
2H), 3.49-3.70 (m, 4H), 1.60-1.77 (m, 6H).

| | | | | | |
|---|---|---|---|---|---|
| 205 | 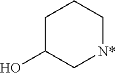 | 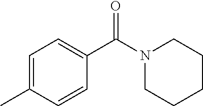 | H | 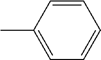 | 498.3 |
| 206 | 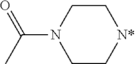 | 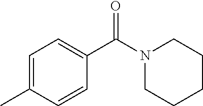 | H | 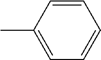 | 525.4 |
| 207 | 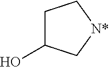 | 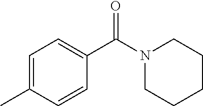 | H | 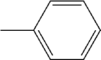 | 484.3 |
| 208 | 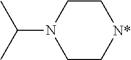 | 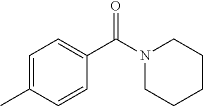 | H | 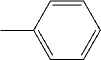 | 525.3 |
| 209 | 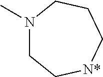 | 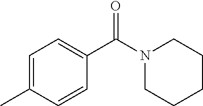 | H | 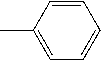 | 511.4 |
| 410 | 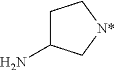 | 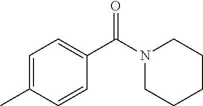 | H | 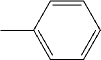 | 483.3 |
| 413 | 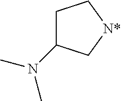 | 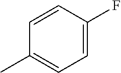 | H | 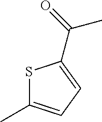 | 466.2 |
| 415 | 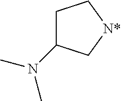 | 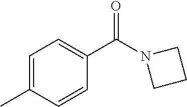 | H | 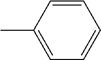 | 483.4 |
| 416 | 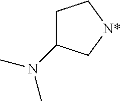 | 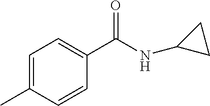 | H | 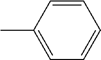 | 483.2 |
| 417 | 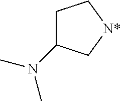 | 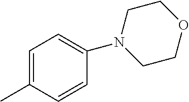 | H | 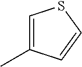 | 491.3 |
| 418 | 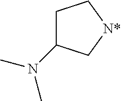 | 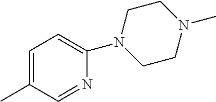 | H | 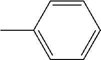 | 499.3 |
| 419 | 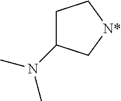 | 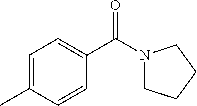 | H | 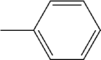 | 497.3 |
| 421 | 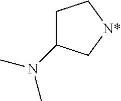 | 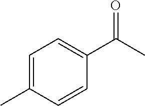 | H | 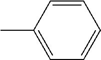 | 442.2 |
| 422 | 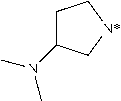 | 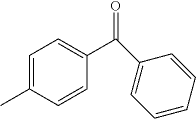 | H | 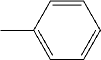 | 504.2 |
| 424 | 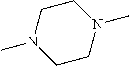 | 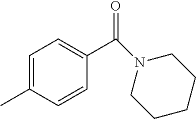 | H | 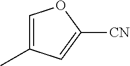 | 512.2 |
| 427 | 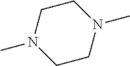 | 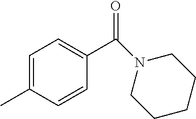 | H | 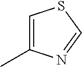 | 504.3 |
| 429 | 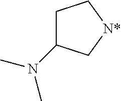 | 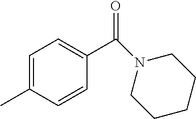 | H | 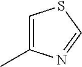 | 518.2 |
| 438 | 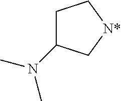 | 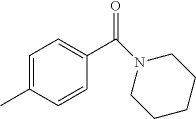 | H | 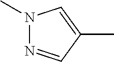 | 515.2 |
| 440 | 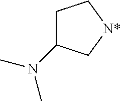 | 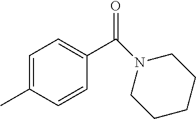 | H | 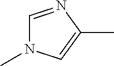 | 515.2 |
| 441 | 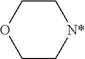 | 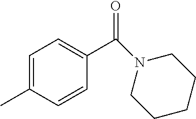 | H | 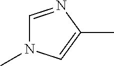 | 488.2 |
| 462 | 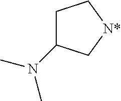 | 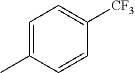 | H | 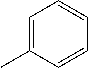 | 468.3 |
| 463 | 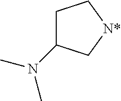 | 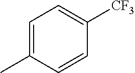 | H | 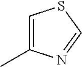 | 475.2 |
| 464 | 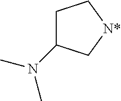 | 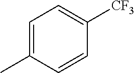 | H | 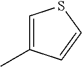 | 474.2 |
| 465 | 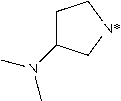 | 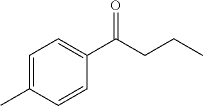 | H | 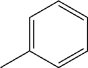 | 470.2 |
| 466 | 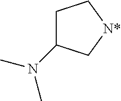 | 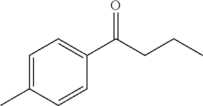 | H | 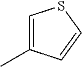 | 476.2 |
| 467 | 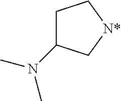 | 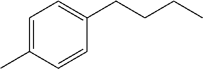 | H | 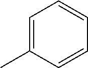 | 456.3 |
| 468 | 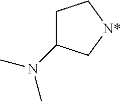 | 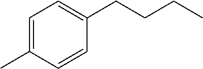 | H | 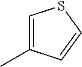 | 462.2 |
| 469 | 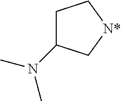 | 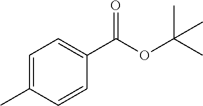 | H | 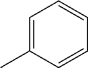 | 500.3 |
| 470 | 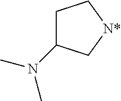 | 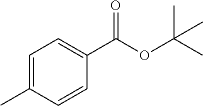 | H | 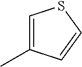 | 506.3 |
| 477 | 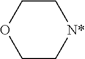 | 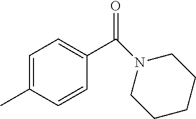 | H | 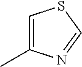 | 491.2 |
| 478 | 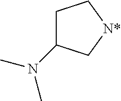 | 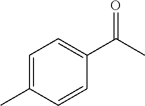 | H | 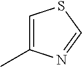 | 449.2 |
| 479 | 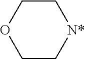 | 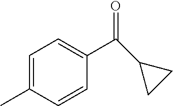 | H | 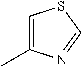 | 448.2 |
| 480 | 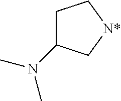 | 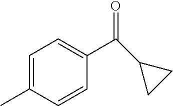 | H | 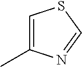 | 475.2 |
| 481 | 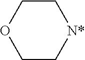 | 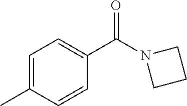 | H | 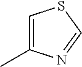 | 463.2 |
| 482 | 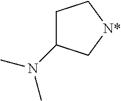 | 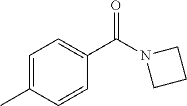 | H | 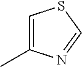 | 490.2 |
| 484 | 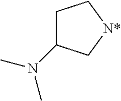 | 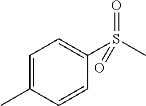 | H | 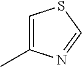 | 485.2 |
| 488 | 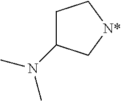 | 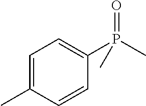 | H | 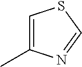 | 483.2 |
| 491 | 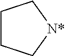 | 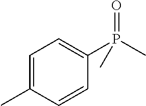 | H | 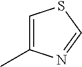 | 440.2 |
| 492 | 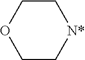 | 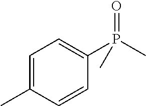 | H | 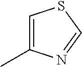 | 456.2 |
| 494 | 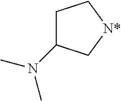 | 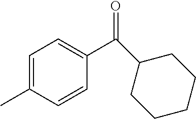 | H | 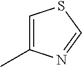 | 517.3 |
| 495 | 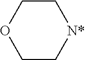 | 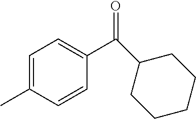 | H | 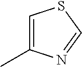 | 490.3 |
| 496 | 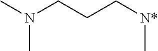 | 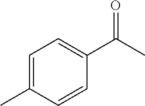 | H | 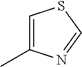 | 451.3 |
| 497 | 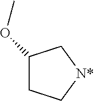 | 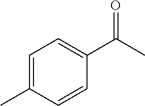 | H | 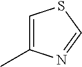 | 436.2 |
| 498 | 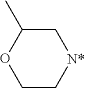 | 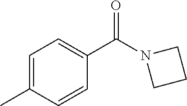 | H | 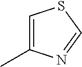 | 476.2 |
| 499 | 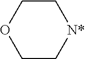 | 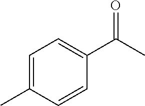 | H | 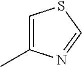 | 421.3 |
| 500 | 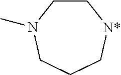 | 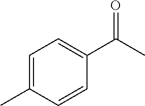 | H | 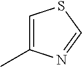 | 449.2 |
| 501 | 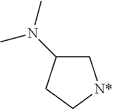 | 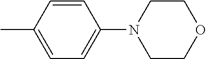 | H | 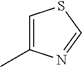 | 492.2 |
| 502 | 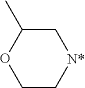 | 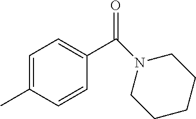 | H | 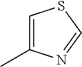 | 504.2 |

Additional Information for Compound 502
¹H NMR 400 MHz (CDCl₃) δ 8.83 (d, 1H, J = 1.6 Hz), 8.67 (s, 1H), 8.21 (d, 1H, J = 2.0 Hz), 7.83 (d, 2H, J = 8.4 Hz), 7.43 (d, 2H,
J = 8.4 Hz), 4.54 (t, 2H, J = 12.8 Hz), 4.07-4.03 (m, 1H), 3.73-3.65 (m, 2H), 3.49-3.46 (m, 4H), 3.20-3.13 (m, 1H), 2.84-2.78 (m, 1H),
1.69-1.46 (m, 6H), 1.30 (d, 3H, J = 6.4 Hz);

| | | | | | |
|---|---|---|---|---|---|
| 503 | 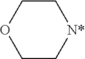 | 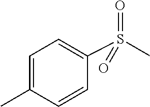 | H | 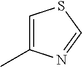 | 458.2 |

Additional Information for Compound 503
¹H NMR 400 MHz (CDCl₃) δ 8.83 (d, 1H, J = 2 Hz), 8.60 (s, 1H), 8.47 (s, 1H), 8.17 (d, 1H, J = 2 Hz), 7.99 (d, 2H, J = 8.8 Hz), 7.93 (d, 2H,
J = 8.8 Hz), 3.89-3.80 (m, 8H), 3.07 (s, 3H);

| | | | | | |
|---|---|---|---|---|---|
| 504 | 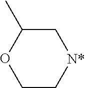 | 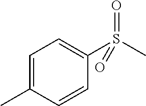 | H | 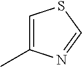 | 472.3 |

Additional Information for Compound 504
¹H NMR 400 MHz (CDCl₃) δ 9.69 (s, 1H), 8.87 (d, 1H, J = 2.4 Hz), 8.83 (s, 1H), 8.26 (d, 1H, J = 2.4 Hz), 8.07 (d, 2H, J = 8.8 Hz), 7.95 (d,
2H, J = 8.8 Hz), 4.53 (t, 2H, J = 10.8 Hz), 4.10-4.07 (m, 1H), 3.74-3.65 (m, 2H), 3.25-3.10 (m, 1H), 3.08 (s, 3H), 2.90-2.84 (m, 1H), 1.33 (d,
3H, J = 6.4 Hz);

| | | | | | |
|---|---|---|---|---|---|
| 505 | 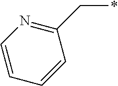 | 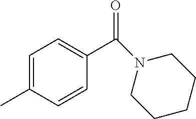 | H | 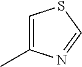 | 511.3 |
| 506 | 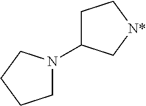 | 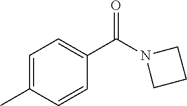 | H | 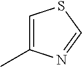 | 516.3 |
| 507 | 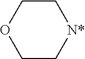 | 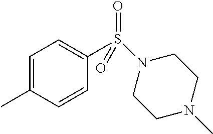 | H | 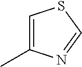 | 542.3 |
| 508 | 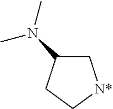 | 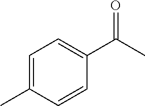 | H | 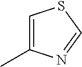 | 449.2 |
| 509 | 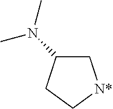 | 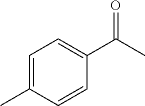 | H | 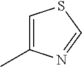 | 449.2 |
| 510 | 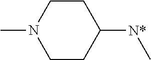 | 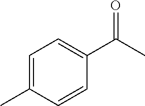 | H | 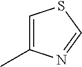 | 463.2 |
| 511 | 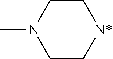 | 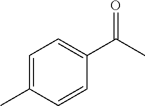 | H | 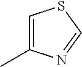 | 435.2 |
| 512 | 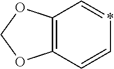 | 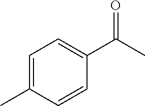 | H | 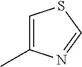 | 457.2 |
| 513 | 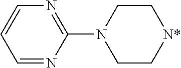 | 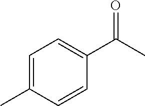 | H | 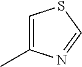 | 499.2 |
| 514 | 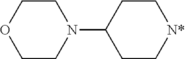 | 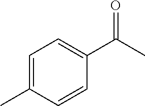 | H | 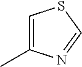 | 505.3 |
| 515 | 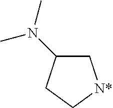 | 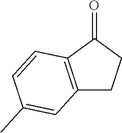 | H | 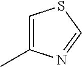 | 461.2 |
| 516 | 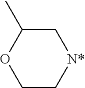 | 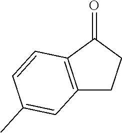 | H | 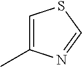 | 448.2 |
| 517 | 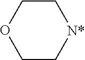 | 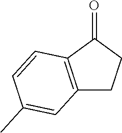 | H | 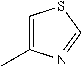 | 434.2 |
| 518 | 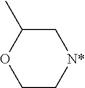 | 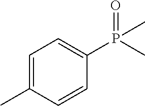 | H | 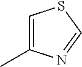 | 470.2 |
| 519 | 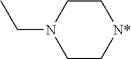 | 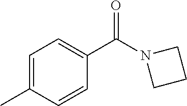 | H | 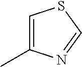 | 490.3 |

Additional Information for Compound 519
¹H NMR 400 MHz (DMSO-d₆) δ 10.22 (s, 1H), 9.65 (s, 1H), 9.30 (d, 1H, J = 2.0 Hz), 8.65 (s, 1H), 8.32 (d, 1H, J = 2.0 Hz), 7.80 (d, 2H,
J = 9.2 Hz), 7.66 (d, 2H, J = 8.8 Hz), 4.81 (d, 2H, J = 15.2 Hz), 4.37 (m, 2H), 4.05 (m, 2H), 3.33 (t, 2H, J = 12.8 Hz), 3.26 (m, 6H), 2.30
(m, 2H), 1.25 (t, 3H, J = 6.8 Hz);

| | | | | | |
|---|---|---|---|---|---|
| 520 | 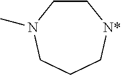 | 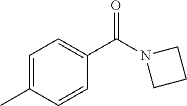 | H | 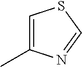 | 490.3 |
| 521 | 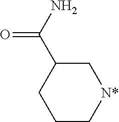 | 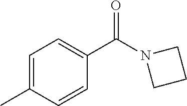 | H | 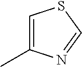 | 504.2 |
| 522 | 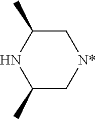 | 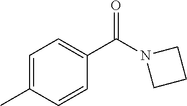 | H | 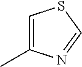 | 490.3 |
| 523 | 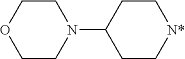 | 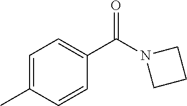 | H | 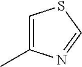 | 546.3 |

Additional Information for Compound 523
¹H NMR 400 MHz (DMSO-d₆) δ 10.22 (s, 1H), 9.74 (s, 1H), 9.40 (d, 1H, J = 2.0 Hz), 8.72 (s, 1H), 8.40 (d, 1H, J = 2.8 Hz), 8.07 (d, 2H,
J = 8.8 Hz), 7.77 (d, 2H, J = 9.2 Hz), 4.96 (d, 2H, J = 13.2 Hz), 4.48 (m, 2H), 4.13 (m, 4H), 3.51 (m, 1H), 3.22 (m, 4H), 2.38 (m, 4H), 1.72
(m, 2H);

| | | | | | |
|---|---|---|---|---|---|
| 524 | 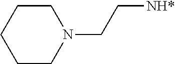 | 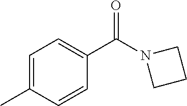 | H | 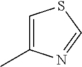 | 504.3 |
| 525 | 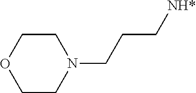 | 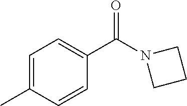 | H | 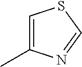 | 520.3 |
| 526 | 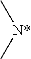 | 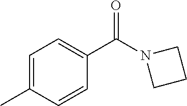 | H | 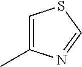 | 421.2 |
| 527 | 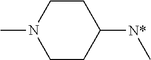 | 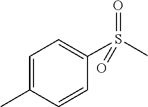 | H | 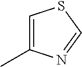 | 499.3 |
| 528 | 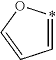 | 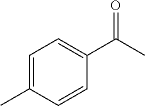 | H | 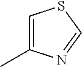 | 403.2 |
| 529 | 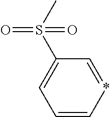 | 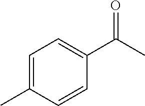 | H | 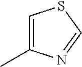 | 491.2 |
| 530 | 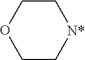 | 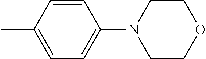 | H | 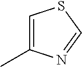 | 465.2 |
| 531 | 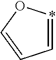 | 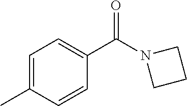 | H | 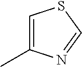 | 444.2 |
| 532 | 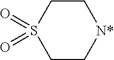 | 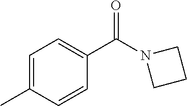 | H | 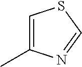 | 511.3 |
| 533 | 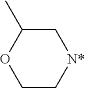 | 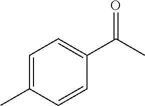 | H | 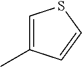 | 435.2 |
| 534 | 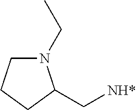 | 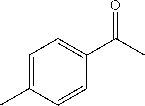 | H | 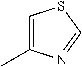 | 463.3 |
| 535 | 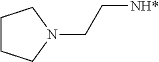 | 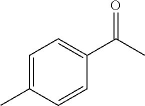 | H | 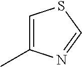 | 449.3 |
| 536 | 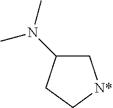 | 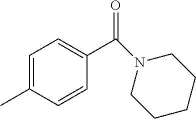 | H | 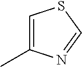 | 524.3 |
| 537 | 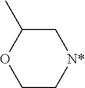 | 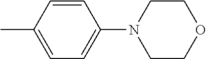 | H | 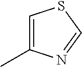 | 479.3 |
| 538 | 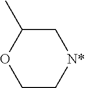 | 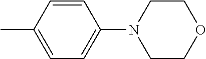 | H | 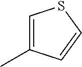 | 478.3 |
| 539 | 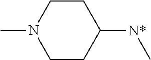 | 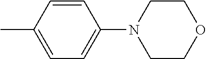 | H | 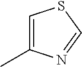 | 506.3 |

Additional Information for Compound 539
¹H NMR 600 MHz (DMSO-d₆) δ 9.59 (s, 1H), 9.27 (d, 1H, J = 2.2), 8.52 (s, 1H), 8.22 (d, 1H, J = 2.0 Hz), 7.77 (d, 2H, J = 8.9 Hz), 6.97 (d,
2H, J = 8.9 Hz), 4.78 (s, 1H), 3.76 (t, 4H, J = 4.6 Hz), 3.57 (t, 4H, J = 4.6 Hz), 3.09 (t, 4H, J = 4.6 Hz), 3.06 (s, 3H), 2.85 (d, 3H, J = 4.6
HZ), 1.96(m, 4H)

| | | | | | |
|---|---|---|---|---|---|
| 540 | 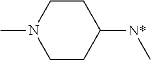 | 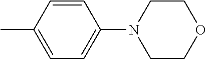 | H | 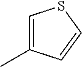 | 505.3 |
| 541 | 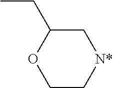 | 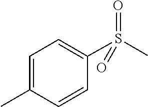 | H | 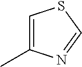 | 486.3 |
| 542 | 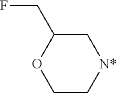 | 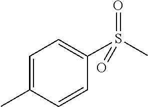 | H | 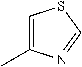 | 490.3 |
| 543 | 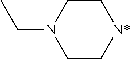 | 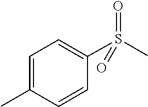 | H | 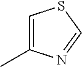 | 485.3 |
| 544 | 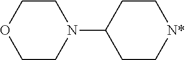 | 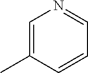 | H | 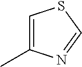 | 464.2 |
| 545 | 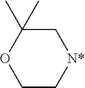 | 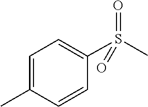 | H | 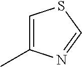 | 486.3 |
| 546 | 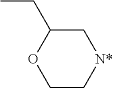 | 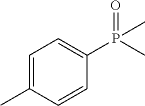 | H | 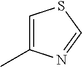 | 484.2 |
| 547 | 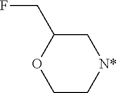 | 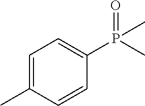 | H | 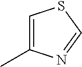 | 488.2 |
| 548 | 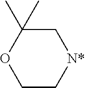 | 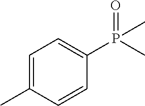 | H | 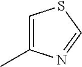 | 484.2 |
| 549 | 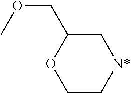 | 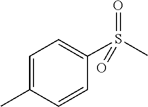 | H | 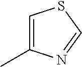 | 502.2 |
| 550 | 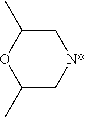 | 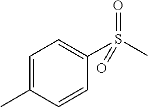 | H | 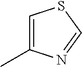 | 486.2 |
| 551 | 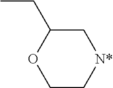 | 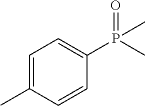 | H | 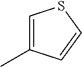 | 483.2 |
| 552 | 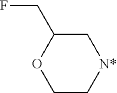 | 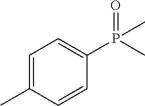 | H | 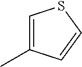 | 487.2 |
| 553 | 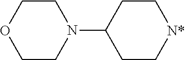 | 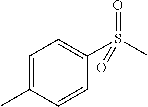 | H | 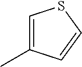 | 540.3 |
| 554 | 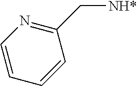 | 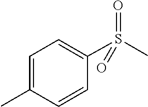 | H | 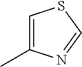 | 479.2 |
| 550 | 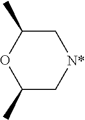 | 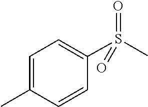 | H | 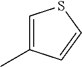 | 485.3 |
| 551 | 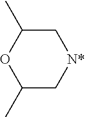 | 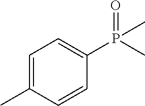 | H | 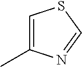 | 484.2 |
| 552 | 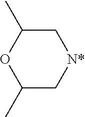 | 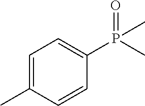 | H | 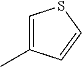 | 483.2 |
| 553 | 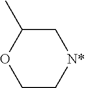 | 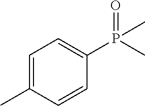 | H | 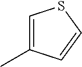 | 469.2 |
| 554 | 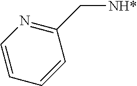 | 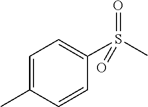 | H | 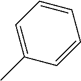 | 472.2 |
| 555 | 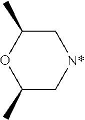 | 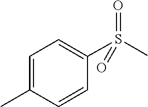 | H | 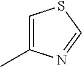 | 486.3 |
| 556 | 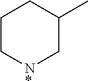 | 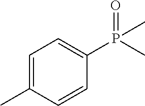 | H | 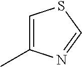 | 468.3 |
| 557 | 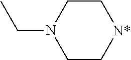 | 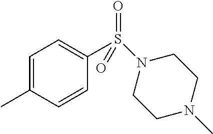 | H | 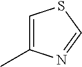 | 569.3 |
| 558 | 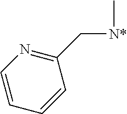 | 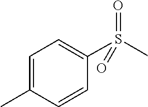 | H | 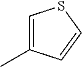 | 492.2 |
| 559 | 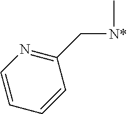 | 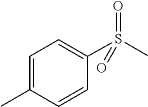 | H | 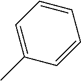 | 486.2 |
| 560 | 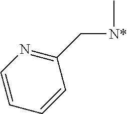 | 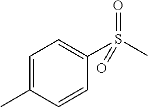 | H | 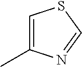 | 493.3 |
| 561 | 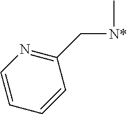 | 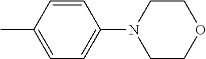 | H | 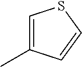 | 499.3 |
| 562 | 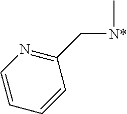 | 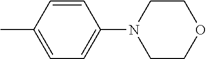 | H | 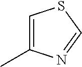 | 500.3 |
| 563 | 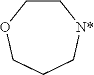 | 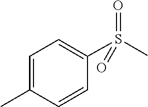 | H | 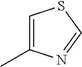 | 472.2 |
| 564 | 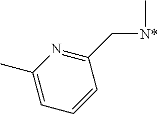 | 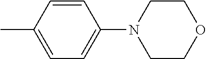 | H | 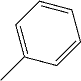 | 507.3 |
| 565 | 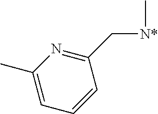 | 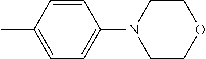 | H | 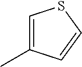 | 513.3 |
| 566 | 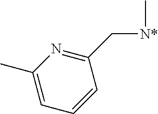 | 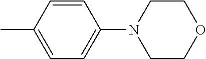 | H | 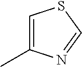 | 514.3 |
| 567 | 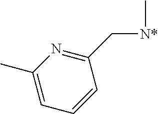 | 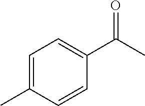 | H | 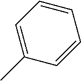 | 464.2 |
| 568 | 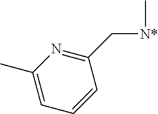 | 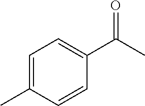 | H | 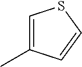 | 470.2 |
| 569 | 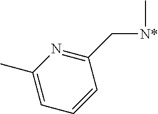 | 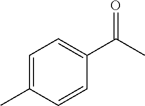 | H | 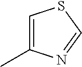 | 471.2 |
| 570 | 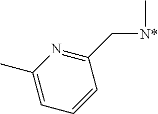 | 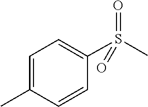 | H | 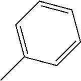 | 500.3 |
| 571 | 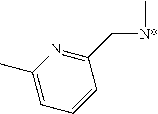 | 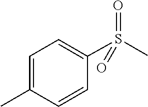 | H | 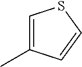 | 503.2 |
| 572 | 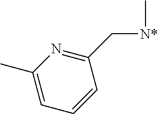 | 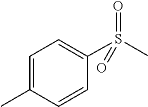 | H | 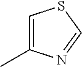 | 507.3 |
| 573 | 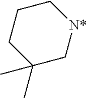 | 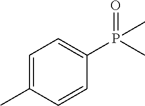 | H | 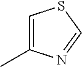 | 482.2 |
| 574 | 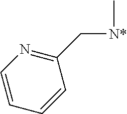 | 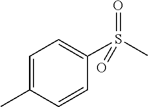 | H | 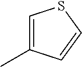 | 492.3 |
| 575 | 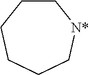 | 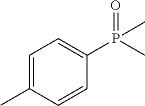 | H | 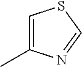 | 468.2 |
| 576 | 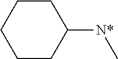 | 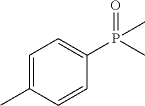 | H | 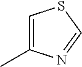 | 482.2 |
| 577 | 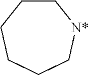 | 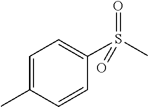 | H | 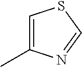 | 470.2 |
| 578 | 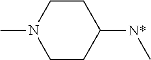 | 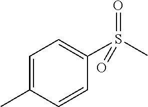 | H | 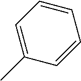 | 492.3 |
| 579 | 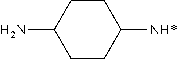 | 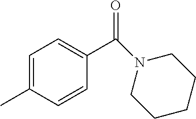 | H | 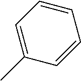 | 511.3 |
| 580 | 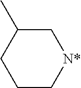 | 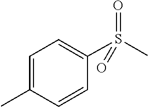 | H | 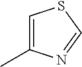 | 470.2 |
| 581 | 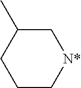 | 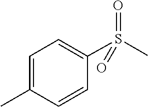 | H | 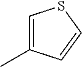 | 469.2 |
| 582 | 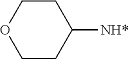 | 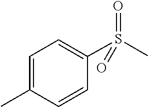 | H | 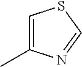 | 472.2 |
| 583 | 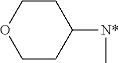 | 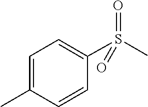 | H | 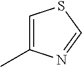 | 486.2 |
| 584 | 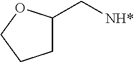 | 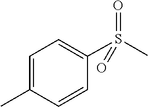 | H | 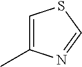 | 472.2 |
| 585 | 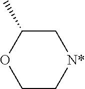 | 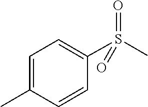 | H | 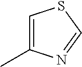 | 472.2 |
| 586 | 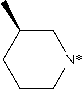 | 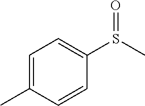 | H | 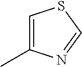 | 454.2 |
| 587 | 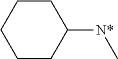 | 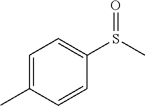 | H | 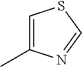 | 467.2 |
| 588 | 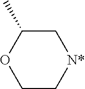 | 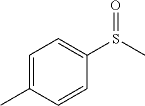 | H | 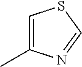 | 456.2 |
| 589 | 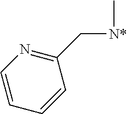 | 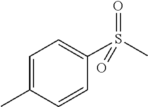 | H | 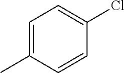 | 520.2 |
| 590 | 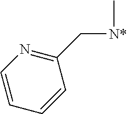 | 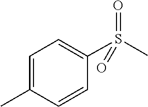 | H | 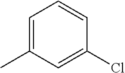 | 520.2 |
| 591 | 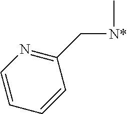 | 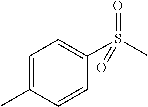 | H | 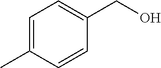 | 516.3 |
| 592 | 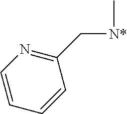 | 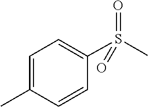 | H | 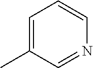 | 487.2 |
| 593 | 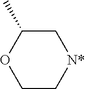 | 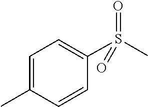 | H | 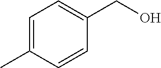 | 495.3 |
| 594 | 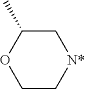 | 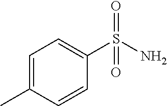 | H | 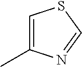 | 473.3 |
| 595 | 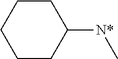 | 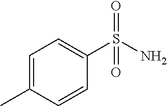 | H | 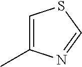 | 485.2 |
| 596 | 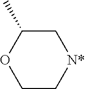 | 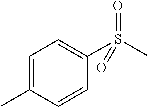 | H | 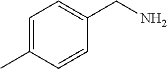 | 494.2 |
| 597 | 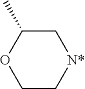 | 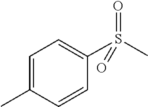 | H | 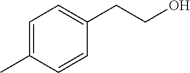 | 509.2 |
| 598 | 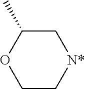 | 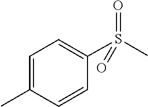 | H | 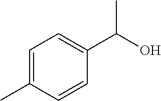 | 509.2 |
| 599 | 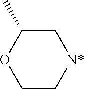 | 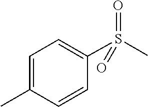 | H | 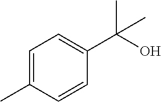 | 523.3 |
| 600 | 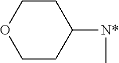 | 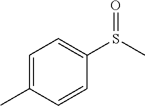 | H | 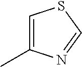 | 470.2 |
| 601 | 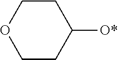 | 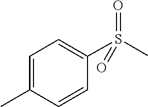 | H | 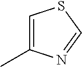 | 473.2 |
| 602 | 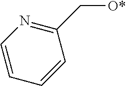 | 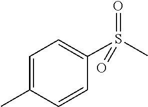 | H | 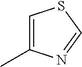 | 480.3 |
| 603 | 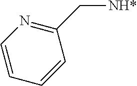 | 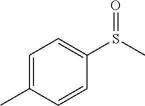 | H | 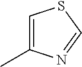 | 463.2 |
| 604 | 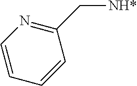 | 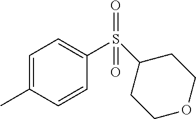 | H | 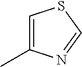 | 549.3 |
| 605 | 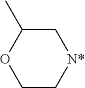 | 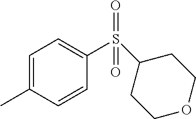 | H | 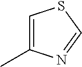 | 541.3 |
| 606 | 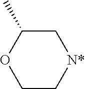 | 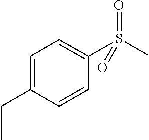 | H | 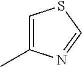 | |
| 607 | 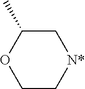 | 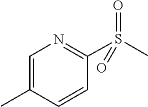 | H | 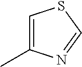 | |
| 608 | 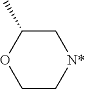 | 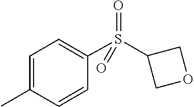 | H | 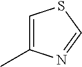 | |
| 609 | 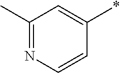 | 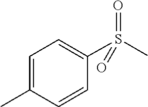 | H | 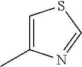 | |
| 610 | 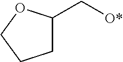 | 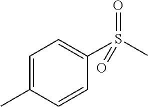 | H | 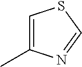 | 473.3 |

The components of Table 3 combine to form compounds of Formula I, for example, the components of compound 605 combine to form [2-(2-Methyl-morpholin-4-yl)-9-thiazol-4-yl-9H-purin-6-yl]-[4-(tetrahydro-pyran-4-sulfonyl)-phenyl]-amine, having the following structure:

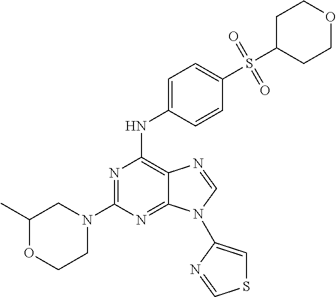

### Assays

The efficacy of compounds of the invention for the treatment of diseases involving deregulated Flt3 and/or FGFR3 receptor tyrosine kinase activity is illustrated by the results of the following pharmacological tests (Examples 10 to 13). These examples illustrate the invention without in any way limiting its scope.

### Example 13

### Flt-3

### Production and Measurement of Activity

The activity is assayed in the presence or absence of different concentrations of inhibitors, by measuring the incorporation of ³³P from γ-³³P-ATP into appropriate substrates.

Tyrosine protein kinase assay with purified GST-Flt-3 is carried out in a final volume of 40 μL containing 500 ng of enzyme in kinase buffer (30 mM Tris-HCl (pH7.5), 3 mM MnCl₂, 15 mM MgCl₂, 1.5 mM DTT, 15 μM Na₃VO₄, 7.5 mg/ml PEG, 0.25 μM poly-EY(Glu, Tyr), 1% DMSO (at highest concentration of compound), 10 μM ATP and γ-³³P-ATP (0.1 μCi)). Two solutions are made: the first solution of 10 μl contains the Flt-3 enzyme and the inhibitor. The second solution contains the substrate (poly-EY), ATP, and γ-³³P-ATP in 30 μl of kinase buffer. Both solutions are mixed on 96-well PVDF filter plates (Millipore, Bedford, Mass., USA), previously wetted with 70% ethanol and rinsed with 1M Tris (7.4). The reaction is incubated at room temperature for 20 minutes, stopped with 0.1% phosphoric acid and then filtered through the plate using a vacuum manifold, allowing the substrate to bind to the membrane. The plates are then washed 5 times with 0.1% phosphoric acid, mounted in Packard TopCount 96-well adapter plate, and 50 μL of Microscint TM (Packard) is added to each well before counting.

IC₅₀ values are calculated by linear regression analysis of the percentage inhibition of each compound (in duplicate) at eight concentrations (1:3 dilution from 1 μM to 0.0005 μM). In this assay, compounds of the invention have an IC₅₀ in the range of 0.1 nM to 2 μM.

### Example 14

The general technique involves comparing the effects of possible inhibitors on cell lines that depend on mutant Flt3 for proliferation vs. cell lines that do not depend on mutant Flt3 for proliferation. Compounds that have differential activity (more than or equal to 10 fold difference in sensitivity between Flt3+ cell lines and Flt3− cell lines are selected for further study.

The cell lines used for the initial screening are sub-lines of Ba/F3 cells that are engineered to over-express mutant or wild-type (non-mutated) Flt3 following infection with a retrovirus expressing appropriate Flt3 cDNAs. The parent cell line, Ba/F3 is dependent on interleukin-3 for proliferation, and when deprived of IL-3, the cells rapidly cease proliferation and die. The retrovirus expresses Flt3 from the retrovirual LTR and the neo gene from an IRES site. Ba/F3 cells are selected in G418 and analyzed for expression of Flt3 by fluorescence activated cell sorting (FACS). Cell lines with two different Flt3 mutations are used. One mutant expresses a Flt-3 that has a 14 amino acid duplication in the juxtamembrane domain encoded by exon 11, the specific duplication being . . . VDFREYEYDLKWEF . . . (termed, Ba/F3-Flt3-ITD). The second mutation has a point mutation that converts asparagines at position 835 to tyrosine (termed Ba/F3-Flt3-D835Y). Both mutations lead to Flt-3 kinase activation and make it independent of IL-3 and the expressing cells grow in the absence of IL-3. Ba/F3 cells expressing wild type Flt3 are similarly generated and used as the “control” cell line. The parental (uninfected) cell line, and the wild-type “control” cell line remain dependent on IL-3 for proliferation.

Ba/F3 cells (-control, -Flt3-ITD, or -Flt3-D835Y) are cultured up to 500,000 cells/mL in 30 mL cultures, with RPMI 1640 with 10% fetal calf serum as the culture medium. The medium for the control cells, (but not the mutant-Flt3 cells) contains 10% conditioned medium from the WEHI-3B cell line as a source of IL-3. A 10 mM “stock” solution of each compound is made in dimethylsufoxide (DMSO). Dilutions are then made into RPMI 1640 with 10% fetal calf serum to create final drug concentrations ranging typically from 1 nM to 10 μM. Similar dilutions are made of DMSO to serve as vehicle controls. 48 hours after addition of compounds, cells are assayed for proliferation rate and cytotoxicity.

Yo-Pro-1 iodide (Molecular Probes) is added to the cells at a final concentration of 2.5 μM in NaCl/Na-citrate buffer. The cells are incubated with Yo-Pro for 10 minutes at room temperature and then read on a fluorimeter for determination of cytotoxicity. Next, the cells are lysed with NP40/EDTA/EGTA buffer, incubated at room temperature for 90 minutes and read for the determination of proliferation.

Compounds that are selectively more toxic to Ba/F3-Flt3-ITD cells than to wild type control Ba/F3 cells are further tested on the Flt3-D835Y expressing cells.

Additionally, α-Flt3 antibodies are used to immunoprecipitate Flt3 proteins before, and after, exposure to various concentrations of active compounds. The immuno-precipitated proteins are separated by sodium dodecyl sulfate polyacrylamide gels, transferred electrophoretically to PVDF membrane, and immunoblotted with an α-phospho-⁵⁹¹Y-Flt3 antibody. This assay determines if compounds reduce the “autophosphorylation” levels of Flt3 characteristic of the mutated forms of the receptor.

Compounds of the invention typically show antiproliferative activity against Flt3-ITD in the nanomolar range while being non-toxic against control-Flt3 up to 10 μM. Compounds of the invention also reduce the autophosphorylation activity of cellular Flt-3 in the nanomolar range.

Compounds of Formula I, in free form or in pharmaceutically acceptable salt form, exhibit valuable pharmacological properties, for example, as indicated by the in vitro tests described in this application. For example, compounds of Formula I preferably show an IC₅₀ in the range of 1×10⁻¹⁰ to 2×10⁻⁶ M, preferably less than 100 nM for Flt3 in the assays described above. For example, {4-[2-(4-amino-cyclohexylamino)-9-thiophen-3-yl-9H-purin-6-ylamino]-phenyl}-piperidin-1-yl-methanone has an IC₅₀ of 5 nM in the assay described by example 14 while showing an IC₅₀ of 7 nM in the assay described in example 13.

### Example 15

### FGFR3

### Measurement of Activity

The activity is assayed in the presence or absence of different concentrations of inhibitors, by measuring the phosphorylation of peptide substrate using HTRF.

Tyrosine protein kinase assay with purified FGFR3 (Upstate) is carried out in a final volume of 10 μL containing 0.25 μg/mL of enzyme in kinase buffer (30 mM Tris-HCl pH7.5, 15 mM MgCl₂, 4.5 mM MnCl₂, 15 μM Na₃VO₄ and 50 μg/mL BSA), and substrates (5 μg/mL biotin-poly-EY(Glu, Tyr) (CIS-US, Inc.) and 3 μM ATP). Two solutions are made: the first solution of 5 μl contains the FGFR3 enzyme in kinase buffer was first dispensed into 384-format Proxiplate® (Perkin-Elmer) followed by adding 50 mL of compounds dissolved in DMSO, then 5 μl of second solution contains the substrate (poly-EY) and ATP in kinase buffer was added to each wells. The reactions are incubated at room temperature for one hour, stopped by adding 10 μL of HTRF detection mixture, which contains 30 mM Tris-HCl pH7.5, 0.5 M KF, 50 mM ETDA, 0.2 mg/mL BSA, 15 μg/mL streptavidin-XL665 (CIS-US, Inc.) and 150 ng/mL cryptate conjugated anti-phosphotyrosine antibody (CIS-US, Inc.). After one hour of room temperature incubation to allow for streptavidin-biotin interaction, time resolved florescent signals are read on Analyst GT (Molecular Devices Corp.).

IC₅₀ values are calculated by linear regression analysis of the percentage inhibition of each compound (in duplicate) at 12 concentrations (1:3 dilution from 10 μM to 0.05 nM). In this assay, compounds of the invention have an IC₅₀ in the range of 0.1 nM to 2 μM.

### Example 16

The general technique involves comparing the effects of possible inhibitors on cell lines that depend on FGFR3 for proliferation vs. cell lines that do not depend on FGFR3 for proliferation. Compounds that have differential activity (more than or equal to 10 fold difference in sensitivity between FGFR3+ cell lines and FGFR3− cell lines are selected for further study.

The cell lines used for the initial screening are sub-lines of Ba/F3 cells that are engineered to over-express TEL-FGFR3 fusion following infection with a retrovirus expressing TEL-FGFR3 cDNAs. The parent cell line, Ba/F3 is dependent on interleukin-3 (IL-3) for proliferation, and when deprived of IL-3, the cells rapidly cease proliferation and die. On the contrary, in the FGFR3 over-expressed Ba/F3 cells, TEL-FGFR3 fusion leads to a ligand-independent FGFR3 dimerization and subsequent FGFR3 kinase activation and that makes over-expressed Ba/F3 cells grow in the absence of IL-3.

Wild type Ba/F3 and transformed Ba/F3 (-TEL-FGFR3) cells are cultured up to 800,000 cells/mL in suspension, with RPMI 1640 supplemented with 10% fetal bovine serum as the culture medium. The medium for the control cells contains 10 ng/ml of recombinant IL-3 (R&D Research). A 10 mM “stock” solution of each compound is made in dimethylsufoxide (DMSO). Dilutions are then made into DMSO create final drug concentrations ranging typically from 0.05 nM to 10 μM. 48 hours after addition of compounds, cells are assayed for proliferation rate. AlamarBlue® (TREK Diagnostic Systems) is added to the cells at a final concentration of 10% in cell culture medium. The cells are incubated with AlamarBlue® for 4 hours in a 37° C. tissue culture incubator and then read on a fluorescence reader for determination of proliferation.

Additionally, phosphorylated TEL-FGFR3 protein levels in over-expressed Ba/F3 lysates after exposure to various concentrations of active compounds are detected in Western blot immunoblotted with anti-phosphorylated-FGFR3 antibody. This assay determines if compounds reduce the “autophosphorylation” levels of FGFR3 characteristic of the mutated forms of the receptor.

Compounds of the invention typically show antiproliferative activity against TEL-FGFR3 in the nanomolar range while being non-toxic against wild type Ba/F3 up to 10 μM. Compounds of the invention also reduce the autophosphorylation activity of cellular TEL-FGFR3 in the nanomolar range.

### Example 17

### Upstate KinaseProfiler™

### Radio-Enzymatic Filter Binding Assay

Compounds of the invention are assessed for their ability to inhibit individual members of a panel of kinases (a partial, non-limiting list of kinases includes: cSRC, Lck, FGFR3, Flt3, TrkB and PFGFRα). The compounds are tested in duplicates at a final concentration of 10 μM following this generic protocol. Note that the kinase buffer composition and the substrates vary for the different kinases included in the “Upstate KinaseProfiler™” panel. The compounds are tested in duplicates at a final concentration of 10 μM following this generic protocol. Note that the kinase buffer composition and the substrates vary for the different kinases included in the “Upstate KinaseProfiler™” panel. Kinase buffer (2.5 μL, 10×—containing MnCl₂ when required), active kinase (0.001-0.01 Units; 2.5 μL), specific or Poly(Glu-4-Tyr) peptide (5-500 μM or 0.01 mg/ml) in kinase buffer and kinase buffer (50 μM; 5 μL) are mixed in an eppendorf on ice. A Mg/ATP mix (10 μL; 67.5 (or 33.75) mM MgCl₂, 450 (or 225) μM ATP and 1 μCi/μl [γ-³²P]-ATP (3000 Ci/mmol)) is added and the reaction is incubated at about 30° C. for about 10 minutes. The reaction mixture is spotted (20 μL) onto a 2 cm×2 cm P81 (phosphocellulose, for positively charged peptide substrates) or Whatman No. 1 (for Poly (Glu-4-Tyr) peptide substrate) paper square. The assay squares are washed 4 times, for 5 minutes each, with 0.75% phosphoric acid and washed once with acetone for 5 minutes. The assay squares are transferred to a scintillation vial, 5 ml scintillation cocktail are added and ³²P incorporation (cpm) to the peptide substrate is quantified with a Beckman scintillation counter. Percentage inhibition is calculated for each reaction.

Compounds of Formula I, at a concentration of 10 μM, preferably show a percentage inhibition of greater than 50%, preferably greater than 60%, more preferably greater than 70%, against cSRC, Lck, FGFR3, Flt3, TrkB and PFGFRα kinases. For example:

(i) Compound 539, N²-Methyl-N²-(1-methyl-piperidin-4-yl)-N⁶-(4-morpholin-4-yl-phenyl)-9-thiazol-4-yl-9H-purine-2,6-diamine shows the following inhibition profile: Bmx (90%), c-Src (97%), Lck (99%), Flt3 (100%), Rsk1 (82%) and TrkB (99%);

(ii) Compound 554 (Example 10), N⁶-(4-Methanesulfonyl-phenyl)-N²-pyridin-2-ylmethyl-9-thiazol-4-yl-9H-purine-2,6-diamine, shows the following inhibition profile: Abl (98%), Bmx (86%), c-Src (99%), Lck (95%), Flt3 (100%), FGFR3 (98%) and TrkB (99%); and

(iii) Compound 503, (4-Methanesulfonyl-phenyl)-(2-morpholin-4-yl-9-thiazol-4-yl -9H-purin-6-yl)-amine, shows the following inhibition profile: Abl (81%), Bmx (71%), c-Src (98%), Lck (99%), Flt3 (99%), TrkB (99%)

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes.

We claim:

1. A compound of Formula I: 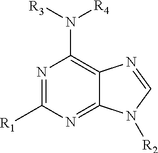
in which:
R₁ is selected from hydrogen, halo, C₁₋₆alkyl, halo-substituted-C₁₋₆alkyl, C₁₋₆alkoxy, halo-substituted-C₁₋₆alkoxy, —OXOR⁵, —OXR⁶, —OXNR₅R₆, —OXONR₅R₆, —XR₆, —XNR₅R₆ and —XNR₇XNR₇R₇; wherein X is selected from a bond, C₁₋₆alkylene, C₂₋₆alkenylene and C₂₋₆alkynylene; wherein R₂ is independently selected from hydrogen or C₁₋₆alkyl;
R₅ is selected from hydrogen, C₁₋₆alkyl and —XOR₇; wherein X is selected from a bond, C₁₋₆alkylene, C₂₋₆alkenylene and C₂₋₆alkynylene; and R₇ is independently selected from hydrogen or C₁₋₆alkyl;
R₆ is selected from hydrogen, C₁₋₆alkyl, C₃₋₁₂cycloalkylC₀₋₄alkyl, C₃₋₈heterocycloalkylC₀₋₄alkyl, C₆₋₁₀arylC₀₋₄alkyl and C₅₋₁₀heteroarylC₀₋₄alkyl; or
R₅ and R₆ together with the nitrogen atom to which both R₅ and R₆ are attached form C₃₋₈heterocycloalkyl or C₅₋₈heteroaryl; wherein a methylene of any heterocycloalkyl formed by R₅ and R₆ can be optionally replaced by —C(O)— or —S(O)₂—;
wherein any aryl, heteroaryl, cycloalkyl or heterocycloalkyl of R₆ or the combination of R₅ and R₆ can be optionally substituted by 1 to 3 radicals independently selected from —XNR₇R₇, —XOR₇, —XNR₇R₇, —XC(O)NR₇R₇, —XNR₇C(O)R₇, —XOR₇, —XC(O)OR₇, —XC(O)R₇, C₁₋₆alkyl, C₃₋₈heterocycloalkyl, C₅₋₁₀heteroaryl, C₃₋₁₂cycloalkyl and C₆₋₁₀arylC₀₋₄alkyl; wherein any alkyl or alkylene of R₁ can optionally have a methylene replaced by a divalent radical selected from —NR₇C(O)—, —C(O)NR₇—, —NR₇—, —C(O)—, —O—, —S—, —S(O)— and —S(O)₂—; and wherein any alkyl or alkylene of R₆ can be optionally substituted by 1 to 3 radicals independently selected from C₅₋₈heteroaryl, —NR₇R₇, —C(O)NR₇R₇, —NR₇C(O)R₇, halo and hydroxy; wherein R₇ is independently selected from hydrogen or C₁₋₆alkyl;
R₂ is selected from hydrogen, C₆₋₁₀aryl and C₅₋₁₀heteroaryl; wherein any aryl or heteroaryl of R₂ is optionally substituted with 1 to 3 radicals independently selected from —XNR₇R₇, —XOR₇, —XOR₈, —XC(O)OR₇, —XC(O)R₇, C₁₋₆alkyl, C₁₋₆alkoxy, nitro, cyano, hydroxy, halo and halo-substituted-C₁₋₆alkyl; wherein X and R₇ are as described above; and R₈ is C₆₋₁₀arylC₀₋₄alkyl;
R₃ is selected from hydrogen and C₁₋₆alkyl;
R₄ is selected from C₃₋₁₂cycloalkylC₀₋₄alkyl, C₃₋₈heterocycloalkylC₀₋₄alkyl, C₆₋₁₀arylC₀₋₄alkyl and C₅₋₁₀heteroarylC₀₋₄alkyl; wherein any alkylene of R₄ can optionally have a methylene replaced by a divalent radical selected from —C(O)—, —S—, —S(O)— and —S(O)₂—; wherein said aryl, heteroaryl, cycloalkyl or heterocycloalkyl of R₄ is optionally substituted by 1 to 3 radicals selected from halo, C₁₋₆alkyl, C₁₋₆alkoxy, halo-substituted-C₁₋₆alkyl, halo-substituted-C₁₋₆alkoxy, —XR₉, —XOR₉, —XS(O)₀₋₂R₇, —XS(O)₀₋₂R₉, —XC(O)R₇, —XC(O)OR₇, —XP(O)R₇R₇, —XC(O)R₉, —XC(O)NR₇XNR₇R₇, —XC(O)NR₇R₇, —XC(O)NR₇R₉ and —XC(O)NR₇XOR₇; wherein X and R₇ are as described above; R₉ is selected from C₃₋₁₂cycloalkylC₀₋₄alkyl, C₃₋₈heterocycloalkylC₀₋₄alkyl, C₆₋₁₀aryl and C₅₋₁₀heteroaryl; wherein any aryl, heteroaryl, cycloalkyl or heterocycloalkyl of R₉ is optionally substituted by 1 to 3 radicals selected from C₁₋₆alkyl, —XC(O)R₇ and —XC(O)NR₇R₇; wherein X and R₇ are as described above; and the pharmaceutically acceptable salts, hydrates, solvates, isomers and prodrugs thereof.

2. The compound of claim 1 in which:
R₁ is selected from hydrogen, halo, C₁₋₆alkoxy, —OXOR⁵, —OXR⁶, —OXNR₅R₆, —OXONR₅R₆, —XR₆, —XNR₇XNR₇R₇ and —XNR₅R₆; wherein X is selected from a bond, C₁₋₆alkylene, C₂₋₆alkenylene and C₂₋₆alkynylene;
R₅ is selected from hydrogen, C₁₋₆alkyl and —XOR₇; wherein X is selected from a bond, C₁₋₆alkylene, C₂₋₆alkenylene and C₂₋₆alkynylene; and R₇ is independently selected from hydrogen or C₁₋₆alkyl;
R₆ is selected from hydrogen, C₁₋₆alkyl, C₃₋₁₂cycloalkylC₀₋₄alkyl, C₃₋₈heterocycloalkylC₀₋₄alkyl, C₆₋₁₀-arylC₀₋₄alkyl and C₅₋₁₀heteroarylC₀₋₄alkyl; R₆ is hydrogen or C₁₋₆alkyl; or
R₅ and R₆ together with the nitrogen atom to which both R₅ and R₆ are attached form C₃₋₈heterocycloalkyl or C₅₋₈heteroaryl; wherein a methylene of any heterocycloalkyl formed by R₅ and R₆ can be optionally replaced by —C(O)— and S(O)₂;
wherein any aryl, heteroaryl, cycloalkyl or heterocycloalkyl of R₆ or the combination of R₅ and R₆ can be optionally substituted by 1 to 3 radicals independently selected from —XNR₇R₇, —XC(O)NR₇R₇, —XOR₇, —XNR₇R₇, —XNR₇C(O)R₇, —XOR₇, —XC(O)R₇, C₁₋₆alkyl, C₃₋₈heterocycloalkyl and C₆₋₁₀-arylC₀₋₄alkyl; wherein any alkyl or alkylene of R₁ can optionally have a methylene replaced by a divalent radical selected from —NR₇C(O)—, —C(O)NR₇—, —NR₇—, —O—; and wherein any alkyl or alkylene of R₁ can be optionally substituted by 1 to 3 radicals independently selected from C₅₋₈heteroaryl, —NR₇R₇, —C(O)NR₇R₇, —NR₇C(O)R₇, halo and hydroxy; wherein R₇ is independently selected from hydrogen or C₁₋₆alkyl;
R₂ is selected from hydrogen, C₆₋₁₀aryl and C₅₋₁₀heteroaryl; wherein any aryl or heteroaryl of R₂ is optionally substituted with 1 to 3 radicals independently selected from —XNR₇R₇, —XOR₇, —XOR₈, —XC(O)OR₇, C₁₋₆alkyl, C₁₋₆alkoxy, nitro, cyano, halo, halo-substituted-C₁₋₆alkoxy and halo-substituted-C₁₋₆alkyl; wherein X and R₇ are as described above; and R₈ is C₆₋₁₀arylC₀₋₄alkyl;
R₃ is hydrogen; and
R₄ is selected from C₆₋₁₀arylC₀₋₄alkyl and C₅₋₁₀heteroarylC₀₋₄alkyl; wherein said aryl or heteroaryl of R₄ is substituted by 1 to 3 radicals selected from halo, —XR₉, —XOR₉, —XS(O)₂R₇, —XS(O)₂R₉, —XC(O)R₇, —XC(O)OR₇, —XP(O)R₇R₇, —XC(O)R₉, —XC(O)NR₇XNR₇R₇, —XC(O)NR₇R₇, —XC(O)NR₇R₉ and —XC(O)NR₇XOR₇; wherein X and R₇ are as described above; R₉ is C₃₋₈heterocycloalkylC₀₋₄alkyl; wherein R₉ is optionally substituted by 1 to 3 radicals selected from C₁₋₆alkyl, —XC(O)R₇ and —XC(O)NR₇R₇; wherein X and R₇ are as described above.

3. The compound of claim 2 in which R₁ is selected from hydrogen, halo, C₁₋₆alkoxy, —OXOR⁵, —OXR⁶, —OXNR₅R₆, —OXONR₅R₆, —XR₆ and —XNR₅R₆; wherein X is selected from a bond, C₁₋₆alkylene, C₂₋₆alkenylene and C₂₋₆alkynylene; R₅ is selected from hydrogen, methyl, hydroxy-ethyl and methoxy-ethyl; R₆ is selected from hydrogen, phenyl, benzyl, cyclopentyl, cyclobutyl, dimethylamino-propenyl, cyclohexyl, 2,3-dihydroxy-propyl, piperidinyl, amino-carbonyl-ethyl, methyl-carbonyl-amino-ethyl, methyl-amino-ethyl, amino-propyl, methyl-amino-propyl, 1-hydroxymethyl-butyl, pentyl, butyl, propyl, methoxy-ethynyl, methoxy-ethenyl, dimethyl-amino-butyl, dimethyl-amino-ethyl, dimethyl-amino-propyl, tetrahydropyranyl, tetrahydrofuranyl-methyl, pyridinyl-methyl, a zepan-1-yl, [1,4]oxazepan-4-yl, piperidinyl-ethyl, diethyl-amino-ethyl, amino-butyl, amino-isopropyl, amino-ethyl, hydroxy-ethyl, 2-acetylamino-ethyl, carbamoyl-ethyl, 4-methyl-[1,4]diazepan-1-yl, 2-hydroxy-propyl, hydroxy-propyl, 2-hydroxy-2-methyl-propyl, methoxy-ethyl, amino-propyl, methyl-amino-propyl, 2-hydroxy-2-phenyl-ethyl, pyridinyl-ethyl, morpholino-propyl, morpholino-ethyl, pyrrolidinyl, pyrrolidinyl-methyl, pyrrolidinyl-ethyl, pyrrolidinyl-propyl, pyrazinyl, quinolin-3-yl, quinolin-5-yl, imidazolyl-ethyl, pyridinyl-methyl, phenethyl, tetrahydro-pyran-4-yl, pyrimidinyl, furanyl, isoxazolyl-methyl, pyridinyl, benzo[1,3]dioxol-5-yl, thiazolyl-ethyl and thiazolyl-methyl; or R₅ and R₆ together with the nitrogen atom to which both R₅ and R₆ are attached form pyrrolidinyl, piperazinyl, piperidinyl, imidazolyl, 3-oxo-piperazin-1-yl, [1,4]diazepan-1-yl, morpholino, 3-oxo-piperazin-1-yl, 1,1-dioxo-1λ⁶-thiomorpholin-4-yl or pyrazolyl;
wherein any aryl, heteroaryl, cycloalkyl or heterocycloalkyl of R₆ or the combination of R₅ and R₆ can be optionally substituted by 1 to 3 radicals independently selected from methyl-carbonyl, amino-methyl, amino-carbonyl, methyl-sulfonyl, methoxy, methoxy-methyl, formyl, fluoro-ethyl, hydroxy-ethyl, amino, dimethyl-amino, hydroxy, methyl, ethyl, acetyl, isopropyl, pyrrolidinyl, pyrimidinyl, morpholino, pyridinyl and benzyl; wherein any alkyl or alkylene of R₆ can optionally have a methylene replaced by a divalent radical selected from —NHC(O)— or —C(O)NH—; and wherein any alkyl or alkylene of R₆ can be optionally substituted by 1 to 2 radicals independently selected from amino, halo, piperidinyl and hydroxy.

4. The compound of claim 2 in which R₂ is selected from hydrogen, phenyl, thienyl, pyridinyl, pyrazolyl, thiazolyl, pyrazinyl, naphthyl, furanyl, benzo[1,3]dioxol-5-yl, isothiazolyl, imidazolyl and pyrimidinyl; wherein any aryl or heteroaryl of R₂ is optionally substituted with 1 to 3 radicals independently selected from methyl, isopropyl, halo, acetyl, trifluoromethyl, nitro, 1-hydroxy-ethyl, 1-hydroxy-1-methyl-ethyl, hydroxy-ethyl, hydroxy-methyl, formamyl, methoxy, benzyloxy, carboxy, amino, cyano, amino-carbonyl, amino-methyl and ethoxy.

5. The compound of claim 2 in which R₄ is selected from phenyl, benzyl, pyridinyl and 1-oxo-indan-5-yl; wherein said phenyl, benzyl, indanyl or pyridinyl is optionally substituted with halo, acetyl, trifluoromethyl, cyclopropyl-amino-carbonyl, azetidine-1-carbonyl, piperidinyl-carbonyl, morpholino, methyl-carbonyl, piperazinyl, methyl-sulfonyl, piperidinyl-sulfonyl, 4-methyl-piperazinyl-carbonyl, dimethyl-amino-ethyl-amino-carbonyl, morpholino-carbonyl, morpholino-methyl, amino-carbonyl, propyl-amino-carbonyl, hydroxy-ethyl-amino-carbonyl, morpholino-ethyl-amino-carbonyl, 4-acetyl-piperazine-1-carbonyl, 4-amino-carbonyl-piperazine-1-carbonyl, phenyl-carbonyl, pyrrolidinyl-1-carbonyl, propyl-carbonyl, butyl, isopropyl-oxy-carbonyl, cyclohexyl-carbonyl, cyclopropyl-carbonyl, methyl-sulfonyl, dimethyl-phosphinoyl, 4-methyl-piperazinyl-sulfonyl, 1-oxo-indan-5-yl, oxetane-3-sulfonyl, amino-sulphonyl and tetrahydro-pyran-4-sulfonyl.

6. The compound of claim 2 selected from: N⁶-(4-Methanesulfinyl-phenyl)-N²-methyl-N²-(tetrahydro-pyran-4-yl)-9-thiazol-4-yl-9H-purine-2,6-diamine; (4-Methanesulfonyl-phenyl)-[2-(2-methyl-morpholin-4-yl)-9-thiazol-4-yl-9H-purin-6-yl]-amine; 1-{4-[2-(2-Methyl-morpholin-4-yl)-9-thiazol-4-yl-9H-purin-6-ylamino]-phenyl}-ethanone; [4-(Dimethyl-phosphinoyl)-phenyl]-[2-(2-methyl-morpholin-4-yl)-9-thiazol-4-yl-9H-purin-6-yl]-amine; Azetidin-1-yl-{4-[2-(4-morpholin-4-yl-piperidin-1-yl)-9-thiazol-4-yl-9H-purin-6-ylamino]-phenyl}-methanone; 1-(4-{2-[Methyl-(1-methyl-piperidin-4-yl)-amino]-9-thiazol-4-yl-9H-purin-6-ylamino}-phenyl)-ethanone; 1-{4-[2-(2-Methyl-morpholin-4-yl)-9-thiophen-3-yl-9H-purin-6-ylamino]-phenyl}-ethanone; (4-Methanesulfonyl-phenyl)-[2-(4-morpholin-4-yl-piperidin-1-yl)-9-thiazol-4-yl-9H-purin-6-yl]-amine; N⁶-(4-Methanesulfonyl-phenyl)-N²-methyl-N²-(1-methyl-piperidin-4-yl)-9-thiazol-4-yl-9H-purine-2,6-diamine; [2-(2-Methyl-morpholin-4-yl)-9-thiazol-4-yl-9H-purin-6-yl]-(4-morpholin-4-yl-phenyl)-amine; N²-Methyl-N²-(1-methyl-piperidin-4-yl)-N⁶-(4-morpholin-4-yl-phenyl)-9-thiazol-4-yl-9H-purine-2,6-diamine; N²-Methyl-N²-(1-methyl-piperidin-4-yl)-N⁶-(4-morpholin-4-yl-phenyl)-9-thiophen-3-yl-9H-purine-2,6-diamine; [2-(2,2-Dimethyl-morpholin-4-yl)-9-thiazol-4-yl-9H-purin-6-yl]-(4-methanesulfonyl-phenyl)-amine; [2-(2,6-Dimethyl-morpholin-4-yl)-9-thiazol-4-yl-9H-purin-6-yl]-(4-methanesulfonyl-phenyl)-amine; [4-(Dimethyl-phosphinoyl)-phenyl]-[2-(2-ethyl-morpholin-4-yl)-9-thiophen-3-yl-9H-purin-6-yl]-amine; [4-(Dimethyl-phosphinoyl)-phenyl]-[2-(2-fluoromethyl-morpholin-4-yl)-9-thiophen-3-yl-9H-purin-6-yl]-amine; [2-(2,6-Dimethyl-morpholin-4-yl)-9-thiazol-4-yl-9H-purin-6-yl]-[4-(dimethyl-phosphinoyl)-phenyl]-amine; [2-(2,6-Dimethyl-morpholin-4-yl)-9-thiophen-3-yl-9H-purin-6-yl]-[4-(dimethyl-phosphinoyl)-phenyl]-amine; [4-(Dimethyl-phosphinoyl)-phenyl]-[2-(2-methyl-morpholin-4-yl)-9-thiophen-3-yl-9H-purin-6-yl]-amine; [4-(Dimethyl-phosphinoyl)-phenyl]-[2-(3-methyl-piperidin-1-yl)-9-thiazol-4-yl-9H-purin-6-yl]-amine; N⁶-(4-Methanesulfonyl-phenyl)-N²-methyl-N²-pyridin-2-ylmethyl-9-thiophen-3-yl-9H-purine-2,6-diamine; N²-Methyl-N⁶-(4-morpholin-4-yl-phenyl)-N²-pyridin-2-ylmethyl-9-thiophen-3-yl-9H-purine-2,6-diamine; (2-Azepan-1-yl-9-thiazol-4-yl-9H-purin-6-yl)-[4-(dimethyl-phosphinoyl)-phenyl]-amine; N²-Cyclohexyl-N⁶-[4-(dimethyl-phosphinoyl)-phenyl]-N²-methyl-9-thiazol-4-yl-9H-purine-2,6-diamine; N⁶-(4-Methanesulfonyl-phenyl)-N²-methyl-N²-(tetrahydro-pyran-4-yl)-9-thiazol-4-yl-9H-purine-2,6-diamine; N⁶-(4-Methanesulfonyl-phenyl)-N²-pyridin-2-ylmethyl-9-thiazol-4-yl-9H-purine-2,6-diamine; N²-Cyclohexyl-N⁶-(4-methanesulfinyl-phenyl)-N²-methyl-9-thiazol-4-yl-9H-purine-2,6-diamine; R-(4-Methanesulfinyl-phenyl)-[2-(2-methyl-morpholin-4-yl)-9-thiazol-4-yl-9H-purin-6-yl]-amine; N⁶-(4-Methanesulfonyl-phenyl)-N²-methyl-N²-pyridin-2-ylmethyl-9-thiazol-4-yl-9H-purine-2,6-diamine; {4-[6-(4-Methanesulfonyl-phenylamino)-2-(methyl-pyridin-2-ylmethyl-amino)-purin-9-yl]-phenyl}-methanol; R-(4-Methanesulfonyl-phenyl)-[2-(2-methyl-morpholin-4-yl)-9-thiazol-4-yl-9H-purin-6-yl]-amine; R-4-[2-(2-Methyl-morpholin-4-yl)-9-thiazol-4-yl-9H-purin-6-ylamino]-benzenesulfonamide; and {4-[6-(4-Methanesulfonyl-phenylamino)-2-(2-methyl-morpholin-4-yl)-purin-9-yl]-phenyl}-methanol.

7. A pharmaceutical composition comprising a therapeutically effective amount of a compound of claim 1 in combination with a pharmaceutically acceptable excipient.

8. A method for treating a disease in an animal in which inhibition of kinase activity can prevent, inhibit or ameliorate the pathology and/or symptomology of the disease, which method comprises administering to the animal a therapeutically effective amount of a compound of claim 1.

9. The method of claim 8 in which the kinase is selected from cSRC, Lck, FGFR3, Flt3, TrkB and Bmx kinases.

10. The use of a compound of claim 1 in the manufacture of a medicament for treating a disease in an animal in which the kinase activity of cSRC, Lck, FGFR3, Flt3, TrkB and/or Bmx contributes to the pathology and/or symptomology of the disease.
